SEQUENCE LISTING (111 sequences)

SEQ ID NO: 1 - DNA, 1578 bp, Oryza sativa
atggcgctctcctccatggccgcggcgcaagagagctccctcctcctcttcctcctcccgacgtcggccgcctccgtgttcccgccgctcatctccgtggtcgtcctcgccgcgctcctcctgtggctctcgccgggtggccccgcgtgggcgctgtcccgttgccgtggcacgccgccgccgccgggcgtggcggggggcgcggccagcgcgctgtccggccctgccgcgcaccgcgtgctcgccgggatttcgcgcgccgtcgagggcggcgcggcggtgatgtcgctctccgtcggcctcacccgcctcgtcgtggcgagccggccggagacggcgagggagatcctcgtcagcccggcgttcggcgaccgccccgtgaaggacgcggcgaggcagctgctgttccaccgcgccatggggttcgccccgtcgggcgacgcgcactggcgcgggctccgccgcgcctccgcggcgcacctcttcggcccgcgccgcgtggccgggtccgcgcccgagcgcgaggccatcggcgcccgcatagtcggcgacgtcgcctccctcatgtcccgccgcggcgaggtccccctccgccgcgtccttcacgccgcgtcgctcggccacgtcatggcgaccgtcttcggcaagcggcacggcgacatctcgatccaggacggcgagctcctggaggagatggtcaccgaagggtacgacctcctcggcaagttcaactgggccgaccacctgccattgctcaggtggctcgacctccagggcatccgccgccggtgcaacaggctagtccagaaggtggaggtgttcgtcggaaagatcatacaggagcacaaggcgaagcgagctgccggaggcgtcgccgtcgccgacggcgtcttgggcgacttcgtcgacgtcctcctcgacctccagggagaggagaagatgtcagactccgacatgatcgctgttctttgggagatgatctttagagggacggacacggtggcgatcttgatggagtgggtgatggcgaggatggtgatgcacccggagatccaggcgaaggcgcaggcggaggtggacgccgccgtggggggacgccgcggcggcgtcgccgacggcgacgtggcgagcctcccctacatccagtccatcgtgaaggagacgctgcgcatgcacccgccgggcccgctcctgtcgtgggcgcgcctcgccgtgcacgacgcgcgcgtcggtggccacgccgtccccgccgggacgacggcgatggtgaacatgtgggcgatcgcccacgacgccgccgtctggccggagccggaggcgttccgcccggagcgcttctcggagggggaggacgtcggcgtgctcggcggcgacctccgcctcgcgccgttcggcgccggccgccgcgtctgccctggcaggatgctggcgctcgccaccgcccacctctggctcgcccagctgctgcacgccttcgactggtcccccaccgccgccggcgtcgacctgtccgagcgcctcggcatgtcgctggagatggcggcgccgctcgtgtgcaaggccgtggctagggcctga

SEQ ID NO: 2 - protein, 525 aa, Oryza sativa
MALSSMAAAQESSLLLFLLPTSAASVFPPLISVVVLAALLLWLSPGGPAWALSRCRGTPPPPGVAGGAASALSGPAAHRVLAGISRAVEGGAAVMSLSVGLTRLVVASRPETAREILVSPAFGDRPVKDAARQLLFHRAMGFAPSGDAHWRGLRRASAAHLFGPRRVAGSAPEREAIGARIVGDVASLMSRRGEVPLRRVLHAASLGHVMATVFGKRHGDISIQDGELLEEMVTEGYDLLGKFNWADHLPLLRWLDLQGIRRRCNRLVQKVEVFVGKIIQEHKAKRAAGGVAVADGVLGDFVDVLLDLQGEEKMSDSDMIAVLWEMIFRGTDTVAILMEWVMARMVMHPEIQAKAQAEVDAAVGGRRGGVADGDVASLPYIQSIVKETLRMHPPGPLLSWARLAVHDARVGGHAVPAGTTAMVNMWAIAHDAAVWPEPEAFRPERFSEGEDVGVLGGDLRLAPFGAGRRVCPGRMLALATAHLWLAQLLHAFDWSPTAAGVDLSERLGMSLEMAAPLVCKAVARA

SEQ ID NO: 3 - DNA, 17201 bp, Oryza sativa
  unsure (12598): n = A, C, G, or T
agggaaattgtagtgttttgcttctcaaaccgctcctgtcttccacttagacttgtaatttcacttctgactttttcgatgtttctctgtaccagtacctgtgcgatctaaacaattgtgtcagtatgtagtgagcagccttaacaaaactgttatcacagtgtgacacattataattgtcttcctttcctgagtatatgtggtcttttggtttgaatgtagaggtcagatttaattcatttctaaagaaaatgtggtcttctagcaacaagctagttgagaaagatggtgaattaaagctaattttcaatctctcaagaaagtaaaccatatgatcatccataatttcctcttaatacgatgatataaatctccacttaagcttctaaatataccattaattatttatgagtactcattttttgtttcggccaattcatagccgctgctactcattatttatgagagtatatatagctagcttgcatctagtgatatgatcgagctagcattcgagccacagctcaaaacgaggccaagatcatacgcgtcgccggatcattcccacacgtgtgagaattgaaccccaaaaaaaaaagagtacggtatttgctagtgcagctaaaagctacgaattgaatatgatatcgatattgtgtagagtatggacgatacatggaatctcatctcatctgatcatcatgatctcctggatgaaaatacaatgtacatgaatagagagagggcttttggttttgggtggagaaatggagcaacactccttgacatttgagccccatcttataatatgaattcaatgaaaaaaaaatggaaaggagaatagagccacgtggcaacaccgacttcgcggaagaggctcgacgaaacgatcttgtgcgtgcgcgtgcagcgatctaggaacgctcttgcgtgcgtgagtgcacgggccaccgggtgtccagaagtttcttcgtgaatatatcgatcgagcaattaggcccatggaccatggctcagcaggccgtgcgatggcacaagaacatgttgggtgatttaggccttgtttagtttctaaaacaaaaacttttcacccatcacatcgaatgtttagaaatatgtgtggagtattaaatgtgaaaaaaaaactcaattacacagtttgcatgtaaattgcgagacaaatcttttaatcctaattgcaccatgatttgacaatgtggtgctacagtaaacatttgctaatgatggattaattaggcttaataaattcgtctcgcggtttcctgacggaatctataatttgtttaattattagactacgtttaatacttcaaatgtgtgtccgtatattcgatgtgacaatcaaacccaatttttttccccaactaaacaagcccttagagagaccaaactttacatggatgaaatgagatattacgcatacatgtaggatgttctatatgcaaacacccgttgcatgctgatcgatgcatgaactttcacattcagtggtccgtactccctactttgtacgcacagctccgattaattatcactttcctcgttccgcattataagatatttattaagcccttcaatccctcgtctagattccctaatatccatatgaatttaaacacatatatgaaacacatacgttgatccatgtatatttttttttcaaaacccaaaacgtattatagtatgaaacataaatttattcaaaacctaaaacatcttatacacatacattgatgcatatatgaatttattaaaaccctaacaaaatagaaatttgttcaaaacccaaaagatcttctatccgattgttaccccaccgggcccacgcctaggctcactaaaccatacgtggcttttgccatgcgcatgcgcttttctagtaatgttaaagtcctagcttgacagtatttgacatcggaagaaattgatgaactgtgtttcgaactagttccaccatttactcttatagcttattgtacgtagccaaaatttaaatttttaaatttatttttgggttttgttccatcgtactttactttttttttcaacatttgcttttaaaccacaaataacacactataacatcatatatatatatatatatatatatgcctcctgattaaaacccggaaatatgatttttgtatttaaatgtgtcctattgatctcctatgctaaatgaatcgtgttttaggctagatatcttttaagatgttactaatttctaatatttaaccaaattttatcataaattctaaatatttatgacataagatagagtagtttgatatagacaagtcaaacccacgtgggataagtgaaagacacatgagtcaagataaactgtgaaatcaataaagggccaagttttacgtgattatcagagatgatagcgggttttactaggttaggcatagagaaaaaagaattatacgatatatgtaacagttttcaaagattctttttatcaaaattcatttattctatttaattatatatatatatagctcaacttgtattatcgctacccgtcaataacattgctcatcgcaataaccaagcagttatcaccgataaagttacaaccctagttaagagacaattagccgtagaatttcactctctttttgtccacaccacttccatcaaaccttaatttggcatctcaattgaaaagttaataacctctccctttttttctgcatgcgatgcgttgctacattgtacatatatacatctatagcaagttcaattggcccgaccgttacgtacgtagagatcgtaataattaacgcacaaagacacaaaatggagggtacagttaacctatatatccagcatccaagcagctggctggcctggctatcaaccacagctgacactaacagctaagctagctaaaagcagccaccggcgaaccgaaggttaaccgtacgtcggcgtcgcggtctcgcggagagccctgagaatgtagagaaaccgatcaccgatgtattattttcctattatgcacatacaatttcagttcttacttgattcaaaattgtttactgcggctatgttttacggtggatagatgtgattacattttttttatatatttgctcttttgttttgaaaaagaaaatcttttgcttactaaattctataactctttcggtggaaggcgacgtaccattgatagcgagacgtgtaggaatttcgttaatcctaatacatgttgaccttttctctaagaagtggttataggagtataaggtctgtatatattcataaggggtgagtatgctttcgtatatgagcatatgcatttgtactatgtttttttttaaaaaaagtggaacattaattcctcgtgatcaaatgtgggacattgactgacatatggatttaataattatttacttgtccacaaataacttaccttgtcatttttactggaggtagatgaactcaaaccattatttataaataatcttttataaatgtcggttccgtacaagccatacgctacagtttcacgtcttaggagatgttagctttttttgcatgcttgacttcacgtgaggaaatgcatgagttttataaatgtatcgtacaagttacaggttataaatgtttattgtttttgaagcggttaaattaaaccacgtaacgactaaagtaagttgcacaactaagatttgcatgcacacaatttgacttgttcctttaatggtgatacataaaaaaaaatcatctgccttacccatgatgaaaataattgaaccacatctaagaaagagtagggattataatgctatgcaattgaattggattgttcaaattctaaatcaaactgttccacttctatctacatgacctctttgtataaattttctcatggtgaaatagtagcaaggtggctaaattaacataggctgctagggaggtcgagtgaggggtatatagagaaaggtcgaggaggaggtagatcattgcggtggacgacatggagatgatcccttctaaactctaaacttgtttcaatcctattctatatagtgaaagtatcatcttttaaggaatcgaaaggttggtctcttaaaaaaaagtttaagataccaccacttttcatgaaatttgactgaatgatgtgctctatatcaaatatttgcatatatatgtcccaaatcaagaccacatatggcaagtgaacaacacacgagtagttcaaaacaaccacggagtcagcggaggaccaacttacacgtgattacagatagaaaaacgagttttactaggtttagatagagtgaaaattttcttttataatgaatctcgacagacagttagtggcgcaacacacaatttaagagacaatcaacaatagaatttcacactcttttttacccacaccacttcacttccattatcgtaaaaccatgatttggcatctcatcaactaaaacgttaacacctctccccttttcccggcgaactgctcgcctggccgatgcatgcaacccgttgctatacattgtacagtacatctatagcaagctagcttccactgctctgccgtttcaattcgcctgtaacgtccagaccgtaataacgcacaaaggcacaaaaatgaaggccaaatggccaattagctagctgtcctggattagtagctgccacagtccacagctaagcagccaccggcaaaccgaaggttagccgtcggcgtcgcgtctggtacgatcgagccctgagaacgtggagaaactgatgtgattatttcctactccatgtatatggacatataatttcagttctttcttgattcaaaaattgtttggtggtgttgtgttttacggtggatagagggttacatatatttatatttgtattttcttgttttgcaaaaaaaaactccctccatcccaaaatataacaattttggggtggatgggacgtaccatagtactatgaatttggacataacccctatccagattcatagtactagaatatgtcccatctacccagaagttgttatattttgagacgggaggagtatttctttgcttattaaattatggaattctttcaatagtaaacgatgtacgtaccctcaagagggagatgcctgtagtgattttgttgatttcaagatacgacaactcactcggtcgaatgtgcttataggggtaggatttgcatgcgttaataaaagtgagtgtgtctgcatatataagcgtctacattagttactatttcaaaaaaaaattgagacattgactgacacgtggatttacttaattatttacttgttcacatataatttagcttgtcggtttttcatcggaggtggattaacttggaccgttatttattaaataatctttatttagaatatgttggttccgtacacatatggtttaacatcttaccagatgctttacgtatacttgatttctacgtgaggaaatacatgagtttcatatctttataattaatgtatcgtacaagtagcatgtatgaaccgtttaatgtttttgtggcggttaaattaaaccacataacgactaaaagtaagttgcattactaagattcgcatgcacataatttggcttgttcctttgatagtaatacttaaaaaaaacattgatcgtcatctgccttactcatgttggaaataactaaattacatctagaaaagataagagcgttaaataggccattcaaatctaaatcaaactgttccacttctatctatatctatatgacctttatgaggcaagttgtcgcatagtgaagatagtagcaaggtggctaaatttacataggtggtcagggaggaggagtttgtcaacaatagggtatagaggaaggtcgaggagtaggtagattgtggtagaagatatggagatgctcccttctaaactagttttaatcctattctatatagtaaaaatatcctcttttaaggaattgaaaggttgatgtccaattcataatatttgattgaatcatgtcctatatattaaacatttatgataagatttttttaaaaaaaatacacaagaagagcatctttgtattaagagaagtaaagtttatttacagataaaacgaaaaatgttttactacctctcttctaaaaagactttattttcttttaccatgaatatacacagtacttaaagaaacaactcgtttattaccacaacactctaccatcaacctttgatttggcatctcaaataaaaaacgctaacctctcccctttccccgggcgcctcttggccgctgcatgcaacccgttgctagtacactgtgtactgctccatctgtagcaagctttcactgctcttccgtttcaattttgcccgttgcatccgtcgagactgaccgtaatgacgcacaaagccaaattagctaagctgtgtcctgcctaagtagagttactaccacagctaagcaagcatcgatcacagccaccggcgaaatgaacggaattaaggttaagatgcagtcaccggcgagatgagtatcctgagaacttggaacaaaccgatgcaaatctctctggccccaactggccatggccatgaattcgtgctcgattccgtgtcattttgcagtagccacccaagagttaattctttcggtttttattccagccttttttttgctttgtttttgtactagctagctagtattatgagactttgcaaaggcgccatactatgtgtattgcaattcaatgcagttttttttctgctgcatttatatttcagttttaatttagcgccacattttgttgctttcctacgtaaagcctggacgcagttaacacagcagctagcttgttagcctgtgacacaatagcaacagctggtaattgtaactgaaaatttctgtttcaaagaagaaaaaaaaagaggtataactggagaaaaaaaagcctggacgatggttttaatcttgttaggtgtgacttaattaccgaatacacaccaaagattgaatgaacactacatgacagtgtcttcctgtgacaggcgttgaaatccctattatggagatggttttcttccttaattcgaaaattgtttggtgccgtcaattagtgaaattgtggacatgttttacggttgacagaggattacatgtatttatgttttatattttcttgtttcacaaaagaatatatatttctttgcttactgaattgtggaatatttttggaaaaaaatacgggacattgagtaatcgacgtgaatatctaattaattatttactatctccgtgcacgagtaacttagcttgtcggttctgactgagaggtagatgtcctttggctgttaatttttttaaaaagcatttctcttttttaatgtcggttccgtacaagctatacacgtggtttcatgtcttggcgctttatcttcgacttccacgtaacaagctgcatgagttttgcgcgcgtctttaaatgttatagtacgtttcatattcgaaccgttaacggtttctgaggcagttaaattaaaccacgtaacgactaaagctgagttgcatgagtaagacccacgcgcactcatttgccttgtttatctagtggtaatacctaaaagaaccgccaatcaaccgccttactcatgttaaaaataattaaattttatcgaggaaagatgaaagataagggtgctatgatactttatatacaatttaattagaccgcaaatcctagatcgaggtgacgccactctatatcgttccacatccgtctatatgatatctttatatgtatgtagttccacattcttatatactcccttccctctggttagttccattttgaactaaccaacgtcaaatttaaaaaaaacagaggtatcatgatattttttaggtttaagttagattgaacggaatggaattgaaatgttgttctcttaattttattttacactatcacatcattacaaatttcaaactcttgttctaaacaggcaccatctttttcagttacatctacactaatttcaatagtaatgccattattatgtagtccaatatttaaggaagaaactaatgatatatatatgcagatattgttaataatggccctttgattacgctatcattactgacaatgacatgtggggccagagtgtcagataattcgaggtccaaatttttggagtggcaaaatggtctatttaaagcaccaggtgtttattagcttctctccacgtcttcttcctcccaagaaaactcctctcacttcgcgaacgcttcccatggcgctctcctccatggccgcggcgcaagagagctccctcctcctcttcctcctcccgacgtcggccgcctccgtgttcccgccgctcatctccgtggtcgtcctcgccgcgctcctcctgtggctctcgccgggtggccccgcgtgggcgctgtcccgttgccgtggcacgccgccgccgccgggcgtggcggggggcgcggccagcgcgctgtccggccctgccgcgcaccgcgtgctcgccgggatttcgcgcgccgtcgagggcggcgcggcggtgatgtcgctctccgtcggcctcacccgcctcgtcgtggcgagccggccggagacggcgagggagatcctcgtcagcccggcgttcggcgaccgccccgtgaaggacgcggcgaggcagctgctgttccaccgcgccatggggttcgccccgtcgggcgacgcgcactggcgcgggctccgccgcgcctccgcggcgcacctcttcggcccgcgccgcgtggccgggtccgcgcccgagcgcgaggccatcggcgcccgcatagtcggcgacgtcgcctccctcatgtcccgccgcggcgaggtccccctccgccgcgtccttcacgccgcgtcgctcggccacgtcatggcgaccgtcttcggcaagcggcacggcgacatctcgatccaggacggcgagctcctggaggagatggtcaccgaagggtacgacctcctcggcaagttcaactgggccgaccacctgccattgctcaggtggctcgacctccagggcatccgccgccggtgcaacaggctagtccagaaggtggaggtgttcgtcggaaagatcatacaggagcacaaggcgaagcgagctgccggaggcgtcgccgtcgccgacggcgtcttgggcgacttcgtcgacgtcctcctcgacctccagggagaggagaagatgtcagactccgacatgatcgctgttctttgggtaagtctcctcgtcgtcgtcttcgtcgtaaagcttgagaaggaaacgtccatggcgttttcatggattggtttcttgtttttttcttcaggagatgatctttagagggacggacacggtggcgatcttgatggagtgggtgatggcgaggatggtgatgcacccggagatccaggcgaaggcgcaggcggaggtggacgccgccgtggggggacgccgcggcggcgtcgccgacggcgacgtggcgagcctcccctacatccagtccatcgtgaaggagacgctgcgcatgcacccgccgggcccgctcctgtcgtgggcgcgcctcgccgtgcacgacgcgcgcgtcggtggccacgccgtccccgccgggacgacggcgatggtgaacatgtgggcgatcgcccacgacgccgccgtctggccggagccggaggcgttccgcccggagcgcttctcggagggggaggacgtcggcgtgctcggcggcgacctccgcctcgcgccgttcggcgccggccgccgcgtctgccctggcaggatgctggcgctcgccaccgcccacctctggctcgcccagctgctgcacgccttcgactggtcccccaccgccgccggcgtcgacctgtccgagcgcctcggcatgtcgctggagatggcggcgccgctcgtgtgcaaggccgtggctagggcctgagccctagccgccgccgccgccattattgccattgatgtggctagcgacgttgtcgtgctcgcatccatactcctccataggcaactcgtctagccaatgaagaaagctactatctatctatctatcaagctagctgctactatcacaaaccgcatttcggcatcatcttaaattagctcttaggggtgtaggcgattttggtttcccccaaaaatttgctttgccagtcttttggtttaaatcgaggcattagttgtgaaacatcatgagaagttatttaaatctgaggaattttgtttgaaccttttctggtgtgctaaatggatcgtgctttgagtatcttattattctgaatgtgttatgtagctacactctcctgaatcatgtgttaaccatgcaatatttctccagttggctgtcagtttatcagcgtcttgtgaatgccgttcatgagaaatctgaccatcttccaaatggtttcatcagtttgctgtgataattaggttatgtttcatgtcagtattatctctgcactgtgtttgttttatacaagtatactgcaacatatataacctttgtacaccatgctagtactgtgacattttcaggttgcatttctttccttttaagactatgaaagattgcgttatgtaacaaacattctattcttctaatatattgacgtgcaatccttttgcgcgttcgagaaaaaaaaaagactatgaaagattaagttactgaacttccactaagtatatggccatatggtctaacctatctctagagattagtcacaaatctgttttgttttgtcaagttgatatccttttttctttctgaatgaaatcaagattatgtccttggaactgcattttgatgctggtctgcattaggctaaatctctgaatctagagccattgcatgctcttgcctgttgcctaattgtagtgctccgagcatcagattcatgtcagcatcaaaacttgcttcttatttcttatcgtcgactcatccttgatcaatgtggccaacaaagatttgtgagcgctaagttgcatccacgtgttgatcatgcatataaacgcaaatgggtcattttctggaatcaagaggatttggccaactcgcttttcgttgtcacaaggtctactactagggtctcatccaaaagattcaacctaagaagatttgatagcaatgtgctgtcgctgttatgttaagattgttaggatcacaatctgtttacagcattacatcctgacagccattctcagtgggactggaagtacaaaacgtggtgttcagaacagtaattttcaaggtagagattgctgatatatatgagaataatttcttggctatcatattaatgttaccaacacaaggtttgtaccttaatcttcatagatttttcatggtgactcgctcatgctagtcatgacttgatgaatatgcaaggagcagtcttcagggatgttactgtcagacagggccaggcatctgaagaccatctgtctaagtgacaggaagtcttcaggcttcagagaacagtcaagattcacttaattaagatggcctgtggctgatctaggtagtcattagtcaaccaaatttcttcatgttccttttcttttccttcctatcttacactaatatagtaacatccagacagtcacgtatcctcctacctttgtgttatggtgagactaactgtgttctggaaggtgtgaaatccctcaccaaaatggctgaagaattgagaattcagaagccatggcagaagtgatcatgtgcatgatgaattgatgataatatatcagggggccctcatctggtcatctcacctgcctctctcttttctctttttctgagacccaaatcttgcataagacttctgtgattagacaggaatcttgtatcctttccccctatggaaagaagcctccattttgtgatatatggctcacatttttattcctgatcaggggcaagatcacaaaaaggtgcttcactgttgacccatcactaccacttttgtggatttgcttgatggcgtgatgcataatttctctatagtcaaaagtcaagcatattttgatagtggttgagaaagtaccgtgaggtaaagtaccttatgctatatcacaagtccataacaccggaaacatataggatgagtttttttcttaactttcccaactcacatctctcgtgttacccgcgcacgtattttaaactgctaaacgatatattttttgcaaaagttttctatacgaaagttgctttaaaaaatcatattaatccatttttcaaaaaaaaagctaatacttaattaatcatacgttaatgagttgctctattttacgtgcatcaaggattagttcccaactgtgtatgccgaacacagccatagttctcaagacacgtaaaaaacataataaacataataattttttgagaatctctaccttcttgaataatctaaattattgcctataattcagcagccaaacgctaaaaaacttagacttttcagatcctcagaagtttgctactcaccatctacttcatacaatctcgagctctcttaaacagggcctcaaggataattttgcctccaaagcctcanaaaaagatacccaaatcctcctcatggcgaccttttgtcaactcttggaacagagaaaatggtcaggtcgtttgtcacacgatcaaacaaagtagagagaaagaaaaaagaaggaaagaaaggatgggattgggttgtttttcccctggacagaaaaagaacagggcccagcccaactaccacgacggcacgacctgaatttgtggttagctgtagatgttttcatggcacaccttccacgtgcaaacttatatatatatatatatatatatatatatatatagagagagagagagagagagagagagagagagagagagagtacttgccaccagcagcttagtgtaattatatgctcgaataataaactgaagaaaaagtgaacaagtggttggtgctgtgtaacacagtattagtgttctttggttgaagattgaaggaagatttagctcgcttttcatgtgcatattttccaaactattaaacggtcttttctaaaaaatatttatatataaagtcgctttaataaaaccatacaagtccatttttcaaatctaaaatgattaatactttattaatcgtatattaatggctaatctcgttttgcgtatctccccaatctttttatttcctttcaaacactacgtcaacttgtattttgtttttccttatttagatggataaacatgtactatatactacaatcccctgttgtcaactggtttcatttgatcattggaggacaatgtaaagaaagtactactttcttcagtcatctttatttatcttcgggatagctaattttaggggggagggggggggggggttggagaaaattcaaaggaaattttataattcttaggaatattttcttattagctcctttggagaaaaggaatacgactgacaaatatcacatgaatttagttctgatcactacaacaaaaatgctttgtagagacatttttctagtactatagatacacttttcaaatgcctttacaatactatagaggcattttaaaaaatgcctaataagtgccttacggtgaattgtctctacaaacgaagaggcattttacaaaatgtctaaaagatggtagaggcattttatagagacattaaattgtgtcacaaccatatgaaaccaatgtaaaaaaaataaaatattttcccttgtttttgacaatccttgaactcatgatcaattgcacaattcattcttatcttcaaggcactaaccaactcaaccctaagtcattacttatatgttgttgtcttgagttatttatatttagtcatttattacatacttttattctaagaagtgcctttacagagtttaaagtgtctcaagaaaatgcctttacatatcaggcacagtttaaagtgccgaaagaatgcctctacaatataaaatctaataaaatatgctgaaaatatttctaaagtgtctgtagagtaaaagttttctaggcattttttaaaatgcctctataaaatgtctctacactataaaactcctgatctaagaggcaatttgcaaaacgcctctacaaaagtgtctttatataaggtttttgttgtagtggatgcctcagttctacaggaatataagtataaacttagacctcatatttttatttttctttgagaagtccgatgcattccctccccttttctctctagtatttttcctcaaaataacttcctccaaaatccctctgaaattccaatgttttatttcctacggacaatccaaatgcataaactcttgaattcgcatgttttaaaatcacttaggaatccaaagtatatatatgacatgatattcatacattctttttctatttatgcgttttgaaaacactatattccaaagagaaacccttagctctcccgacgtcaaataagagtgaccgttctcgcattcactccatcgcactacttcatgccgcaaaatgtttccatttgaaattattgtttatttatacatacgacccacgcccgactcaactattgcatagacactactgttattttcctagacccacatagagataaactcagtgcaaggattagtggatagagatgcgatcgaatgttagtcgtacgtcatggtcgtatatagtaggccgtcatgacattagtggaacgtatggcaccctcaatatatattttttctatgaaagctgtcctctttgggagcccgatgtgaaggaaaaaatatcatgctagctttctttctgacccattcctctccctcctcctactccactcccgtagcttgtgtcgcatgaggtggagctcatttggttggcaagggagacgtcgaccggactttgtcctcggaactaggattctctttttcactaacatgtgagtccgataaatcctagacccacatggtagtgacaaaaaaaaacatggcaactttgaaggtagaggatctcaatctttgatgagcttctttctcatcctctattgtcactagagctcatttggttgggacgatgccatccattagatttggtgacatcccgagggacaaaagcggttagggggtagggaggtcagacactagagatggtacggggcaatggcgtggtggctagcgtcagggaaaataatatggagacaacaccgtacgatgacatttaccttgagccctcagatttaaggctgcgtggatttcctcgggaggacatcgtcacctcatcaccgggagcatacaagagagaagagtggatatgcgcgttgtgaattttcgatgtttcaggcagcacatacggatgtttctcgtatttcgatcaaaatgttaaagtggggattttgatggcgtttctttttttttttttgtggcacagttcctcagcaagacaagcgcacggcttcacccactcacctactacctctgcgttgtttcgccccgtctgctagcgcggcgggtcgttgtcttcttcatcaacaggaggcggcaagtagccaagtaggaggcatccccatagtcgcgcaaccttacctccggatcttcgtattatattgttttatattgtttcttcttcttcttcttcttcttcttcttcttcttcttcttgtttgtgtagcaagtagcaacggagtctcagatcagattagccgccacaggggaggggagaccatggacgaggccgccgccggccaacgcgccagtcctcttcttgccaaggtacggcgaaccgcggaaactgctaatccccgcaggcgcctatcctgaccttttcctcttgtatatatgtgtgtttcttgctttgctgccttatggggtttcaggggaagcagaatagtatgatgcaaagattgtggctttaccgatcaagatttggtttttactacagttgggtgtggggatgtgagctggagtaaatttttttgttgttgtttttttttttaaagaagaaaatgcaaatcgtagtctgaaattgagaaaagaaaaatgcgatgactgataactgctacctggatactgatctttgatgttgataaggattatgaaaccccgaagatgtctagtgcgtatttcattccgtcggtggatttggtcaaagaatgttgagttttagattgttgtaccttgttttggaacgcgaaattttgcagatcgaataagctgttttcgatctactcaatcacattgcggtgtttatgctgacgtctggccttctgtcagaatgatggatcaagctatggtgaagaatcacagagtttattggaagaacaggagccacaggttaaaactaaacaatctggctggagagcaccatcaatcattctgggtgagccttgttatataagcatacccttcttctcgtaaaatcaaaatcttctctccatcgaaaactgtgtgaaaaccaattcatatatagagggattggagcacccatcggtgcatcactatataaacattctttaccatgatgccacaactaatgctcacaaatcatgcaggacttgaatgcttggagagcatggctttcaatggcattgccacaaatctagttgtgtatattcgctcagttctccatggtggcatcgcttccagtgcttcaacttcttctctttggtacggtactagtttctttgtgcctatacttggagcaaccattgcagatacttactggggaaactataagacagtcttgatctcctttatcatgtatttacttgtaagatcagttttcctgctcaactgctcaatcttattcacatttcattgaacaattgaactactccagagtcaagatgatgcatttttgttgtagaaaggtcataatgaaataccgatgcacatttcagggtacggtattcattactgttggagcttttctgccttctgctccagccttatgcaacacggaatcatgctcatcaatgaatgggactcaacatctagtatacttctcnaggcctgtatctcactgctattggttgtggcggagtaaggtctgcgttgcttccgcttggtgcagatcaattcaacaacgatagcagtttagatatacaaaagagaaggaattcttcagtttattctacatttgtgttatctttggtgtgatacttctgg

SEQ ID NO: 4 - DNA, 8300 bp, Oryza sativa
agggaaattgtagtgttttgcttctcaaaccgctcctgtcttccacttagacttgtaatttcacttctgactttttcgatgtttctctgtaccagtacctgtgcgatctaaacaattgtgtcagtatgtagtgagcagccttaacaaaactgttatcacagtgtgacacattataattgtcttcctttcctgagtatatgtggtcttttggtttgaatgtagaggtcagatttaattcatttctaaagaaaatgtggtcttctagcaacaagctagttgagaaagatggtgaattaaagctaattttcaatctctcaagaaagtaaaccatatgatcatccataatttcctcttaatacgatgatataaatctccacttaagcttctaaatataccattaattatttatgagtactcattttttgtttcggccaattcatagccgctgctactcattatttatgagagtatatatagctagcttgcatctagtgatatgatcgagctagcattcgagccacagctcaaaacgaggccaagatcatacgcgtcgccggatcattcccacacgtgtgagaattgaaccccaaaaaaaaaagagtacggtatttgctagtgcagctaaaagctacgaattgaatatgatatcgatattgtgtagagtatggacgatacatggaatctcatctcatctgatcatcatgatctcctggatgaaaatacaatgtacatgaatagagagagggcttttggttttgggtggagaaatggagcaacactccttgacatttgagccccatcttataatatgaattcaatgaaaaaaaaatggaaaggagaatagagccacgtggcaacaccgacttcgcggaagaggctcgacgaaacgatcttgtgcgtgcgcgtgcagcgatctaggaacgctcttgcgtgcgtgagtgcacgggccaccgggtgtccagaagtttcttcgtgaatatatcgatcgagcaattaggcccatggaccatggctcagcaggccgtgcgatggcacaagaacatgttgggtgatttaggccttgtttagtttctaaaacaaaaacttttcacccatcacatcgaatgtttagaaatatgtgtggagtattaaatgtgaaaaaaaaactcaattacacagtttgcatgtaaattgcgagacaaatcttttaatcctaattgcaccatgatttgacaatgtggtgctacagtaaacatttgctaatgatggattaattaggcttaataaattcgtctcgcggtttcctgacggaatctataatttgtttaattattagactacgtttaatacttcaaatgtgtgtccgtatattcgatgtgacaatcaaacccaatttttttccccaactaaacaagcccttagagagaccaaactttacatggatgaaatgagatattacgcatacatgtaggatgttctatatgcaaacacccgttgcatgctgatcgatgcatgaactttcacattcagtggtccgtactccctactttgtacgcacagctccgattaattatcactttcctcgttccgcattataagatatttattaagcccttcaatccctcgtctagattccctaatatccatatgaatttaaacacatatatgaaacacatacgttgatccatgtatatttttttttcaaaacccaaaacgtattatagtatgaaacataaatttattcaaaacctaaaacatcttatacacatacattgatgcatatatgaatttattaaaaccctaacaaaatagaaatttgttcaaaacccaaaagatcttctatccgattgttaccccaccgggcccacgcctaggctcactaaaccatacgtggcttttgccatgcgcatgcgcttttctagtaatgttaaagtcctagcttgacagtatttgacatcggaagaaattgatgaactgtgtttcgaactagttccaccatttactcttatagcttattgtacgtagccaaaatttaaatttttaaatttatttttgggttttgttccatcgtactttactttttttttcaacatttgcttttaaaccacaaataacacactataacatcatatatatatatatatatatatatgcctcctgattaaaacccggaaatatgatttttgtatttaaatgtgtcctattgatctcctatgctaaatgaatcgtgttttaggctagatatcttttaagatgttactaatttctaatatttaaccaaattttatcataaattctaaatatttatgacataagatagagtagtttgatatagacaagtcaaacccacgtgggataagtgaaagacacatgagtcaagataaactgtgaaatcaataaagggccaagttttacgtgattatcagagatgatagcgggttttactaggttaggcatagagaaaaaagaattatacgatatatgtaacagttttcaaagattctttttatcaaaattcatttattctatttaattatatatatatatagctcaacttgtattatcgctacccgtcaataacattgctcatcgcaataaccaagcagttatcaccgataaagttacaaccctagttaagagacaattagccgtagaatttcactctctttttgtccacaccacttccatcaaaccttaatttggcatctcaattgaaaagttaataacctctccctttttttctgcatgcgatgcgttgctacattgtacatatatacatctatagcaagttcaattggcccgaccgttacgtacgtagagatcgtaataattaacgcacaaagacacaaaatggagggtacagttaacctatatatccagcatccaagcagctggctggcctggctatcaaccacagctgacactaacagctaagctagctaaaagcagccaccggcgaaccgaaggttaaccgtacgtcggcgtcgcggtctcgcggagagccctgagaatgtagagaaaccgatcaccgatgtattattttcctattatgcacatacaatttcagttcttacttgattcaaaattgtttactgcggctatgttttacggtggatagatgtgattacattttttttatatatttgctcttttgttttgaaaaagaaaatcttttgcttactaaattctataactctttcggtggaaggcgacgtaccattgatagcgagacgtgtaggaatttcgttaatcctaatacatgttgaccttttctctaagaagtggttataggagtataaggtctgtatatattcataaggggtgagtatgctttcgtatatgagcatatgcatttgtactatgtttttttttaaaaaaagtggaacattaattcctcgtgatcaaatgtgggacattgactgacatatggatttaataattatttacttgtccacaaataacttaccttgtcatttttactggaggtagatgaactcaaaccattatttataaataatcttttataaatgtcggttccgtacaagccatacgctacagtttcacgtcttaggagatgttagctttttttgcatgcttgacttcacgtgaggaaatgcatgagttttataaatgtatcgtacaagttacaggttataaatgtttattgtttttgaagcggttaaattaaaccacgtaacgactaaagtaagttgcacaactaagatttgcatgcacacaatttgacttgttcctttaatggtgatacataaaaaaaaatcatctgccttacccatgatgaaaataattgaaccacatctaagaaagagtagggattataatgctatgcaattgaattggattgttcaaattctaaatcaaactgttccacttctatctacatgacctctttgtataaattttctcatggtgaaatagtagcaaggtggctaaattaacataggctgctagggaggtcgagtgaggggtatatagagaaaggtcgaggaggaggtagatcattgcggtggacgacatggagatgatcccttctaaactctaaacttgtttcaatcctattctatatagtgaaagtatcatcttttaaggaatcgaaaggttggtctcttaaaaaaaagtttaagataccaccacttttcatgaaatttgactgaatgatgtgctctatatcaaatatttgcatatatatgtcccaaatcaagaccacatatggcaagtgaacaacacacgagtagttcaaaacaaccacggagtcagcggaggaccaacttacacgtgattacagatagaaaaacgagttttactaggtttagatagagtgaaaattttcttttataatgaatctcgacagacagttagtggcgcaacacacaatttaagagacaatcaacaatagaatttcacactcttttttacccacaccacttcacttccattatcgtaaaaccatgatttggcatctcatcaactaaaacgttaacacctctccccttttcccggcgaactgctcgcctggccgatgcatgcaacccgttgctatacattgtacagtacatctatagcaagctagcttccactgctctgccgtttcaattcgcctgtaacgtccagaccgtaataacgcacaaaggcacaaaaatgaaggccaaatggccaattagctagctgtcctggattagtagctgccacagtccacagctaagcagccaccggcaaaccgaaggttagccgtcggcgtcgcgtctggtacgatcgagccctgagaacgtggagaaactgatgtgattatttcctactccatgtatatggacatataatttcagttctttcttgattcaaaaattgtttggtggtgttgtgttttacggtggatagagggttacatatatttatatttgtattttcttgttttgcaaaaaaaaactccctccatcccaaaatataacaattttggggtggatgggacgtaccatagtactatgaatttggacataacccctatccagattcatagtactagaatatgtcccatctacccagaagttgttatattttgagacgggaggagtatttctttgcttattaaattatggaattctttcaatagtaaacgatgtacgtaccctcaagagggagatgcctgtagtgattttgttgatttcaagatacgacaactcactcggtcgaatgtgcttataggggtaggatttgcatgcgttaataaaagtgagtgtgtctgcatatataagcgtctacattagttactatttcaaaaaaaaattgagacattgactgacacgtggatttacttaattatttacttgttcacatataatttagcttgtcggtttttcatcggaggtggattaacttggaccgttatttattaaataatctttatttagaatatgttggttccgtacacatatggtttaacatcttaccagatgctttacgtatacttgatttctacgtgaggaaatacatgagtttcatatctttataattaatgtatcgtacaagtagcatgtatgaaccgtttaatgtttttgtggcggttaaattaaaccacataacgactaaaagtaagttgcattactaagattcgcatgcacataatttggcttgttcctttgatagtaatacttaaaaaaaacattgatcgtcatctgccttactcatgttggaaataactaaattacatctagaaaagataagagcgttaaataggccattcaaatctaaatcaaactgttccacttctatctatatctatatgacctttatgaggcaagttgtcgcatagtgaagatagtagcaaggtggctaaatttacataggtggtcagggaggaggagtttgtcaacaatagggtatagaggaaggtcgaggagtaggtagattgtggtagaagatatggagatgctcccttctaaactagttttaatcctattctatatagtaaaaatatcctcttttaaggaattgaaaggttgatgtccaattcataatatttgattgaatcatgtcctatatattaaacatttatgataagatttttttaaaaaaaatacacaagaagagcatctttgtattaagagaagtaaagtttatttacagataaaacgaaaaatgttttactacctctcttctaaaaagactttattttcttttaccatgaatatacacagtacttaaagaaacaactcgtttattaccacaacactctaccatcaacctttgatttggcatctcaaataaaaaacgctaacctctcccctttccccgggcgcctcttggccgctgcatgcaacccgttgctagtacactgtgtactgctccatctgtagcaagctttcactgctcttccgtttcaattttgcccgttgcatccgtcgagactgaccgtaatgacgcacaaagccaaattagctaagctgtgtcctgcctaagtagagttactaccacagctaagcaagcatcgatcacagccaccggcgaaatgaacggaattaaggttaagatgcagtcaccggcgagatgagtatcctgagaacttggaacaaaccgatgcaaatctctctggccccaactggccatggccatgaattcgtgctcgattccgtgtcattttgcagtagccacccaagagttaattctttcggtttttattccagccttttttttgctttgtttttgtactagctagctagtattatgagactttgcaaaggcgccatactatgtgtattgcaattcaatgcagttttttttctgctgcatttatatttcagttttaatttagcgccacattttgttgctttcctacgtaaagcctggacgcagttaacacagcagctagcttgttagcctgtgacacaatagcaacagctggtaattgtaactgaaaatttctgtttcaaagaagaaaaaaaaagaggtataactggagaaaaaaaagcctggacgatggttttaatcttgttaggtgtgacttaattaccgaatacacaccaaagattgaatgaacactacatgacagtgtcttcctgtgacaggcgttgaaatccctattatggagatggttttcttccttaattcgaaaattgtttggtgccgtcaattagtgaaattgtggacatgttttacggttgacagaggattacatgtatttatgttttatattttcttgtttcacaaaagaatatatatttctttgcttactgaattgtggaatatttttggaaaaaaatacgggacattgagtaatcgacgtgaatatctaattaattatttactatctccgtgcacgagtaacttagcttgtcggttctgactgagaggtagatgtcctttggctgttaatttttttaaaaagcatttctcttttttaatgtcggttccgtacaagctatacacgtggtttcatgtcttggcgctttatcttcgacttccacgtaacaagctgcatgagttttgcgcgcgtctttaaatgttatagtacgtttcatattcgaaccgttaacggtttctgaggcagttaaattaaaccacgtaacgactaaagctgagttgcatgagtaagacccacgcgcactcatttgccttgtttatctagtggtaatacctaaaagaaccgccaatcaaccgccttactcatgttaaaaataattaaattttatcgaggaaagatgaaagataagggtgctatgatactttatatacaatttaattagaccgcaaatcctagatcgaggtgacgccactctatatcgttccacatccgtctatatgatatctttatatgtatgtagttccacattcttatatactcccttccctctggttagttccattttgaactaaccaacgtcaaatttaaaaaaaacagaggtatcatgatattttttaggtttaagttagattgaacggaatggaattgaaatgttgttctcttaattttattttacactatcacatcattacaaatttcaaactcttgttctaaacaggcaccatctttttcagttacatctacactaatttcaatagtaatgccattattatgtagtccaatatttaaggaagaaactaatgatatatatatgcagatattgttaataatggccctttgattacgctatcattactgacaatgacatgtggggccagagtgtcagataattcgaggtccaaatttttggagtggcaaaatggtctatttaaagcaccaggtgtttattagcttctctccacgtcttcttcctcccaagaaaactcctctcacttcgcgaacgcttccc

SEQ ID NO: 5 - DNA, 7232 bp, Oryza sativa
  unsure (2629): n = A, C, G, or T
gccctagccgccgccgccgccattattgccattgatgtggctagcgacgttgtcgtgctcgcatccatactcctccataggcaactcgtctagccaatgaagaaagctactatctatctatctatcaagctagctgctactatcacaaaccgcatttcggcatcatcttaaattagctcttaggggtgtaggcgattttggtttcccccaaaaatttgctttgccagtcttttggtttaaatcgaggcattagttgtgaaacatcatgagaagttatttaaatctgaggaattttgtttgaaccttttctggtgtgctaaatggatcgtgctttgagtatcttattattctgaatgtgttatgtagctacactctcctgaatcatgtgttaaccatgcaatatttctccagttggctgtcagtttatcagcgtcttgtgaatgccgttcatgagaaatctgaccatcttccaaatggtttcatcagtttgctgtgataattaggttatgtttcatgtcagtattatctctgcactgtgtttgttttatacaagtatactgcaacatatataacctttgtacaccatgctagtactgtgacattttcaggttgcatttctttccttttaagactatgaaagattgcgttatgtaacaaacattctattcttctaatatattgacgtgcaatccttttgcgcgttcgagaaaaaaaaaagactatgaaagattaagttactgaacttccactaagtatatggccatatggtctaacctatctctagagattagtcacaaatctgttttgttttgtcaagttgatatccttttttctttctgaatgaaatcaagattatgtccttggaactgcattttgatgctggtctgcattaggctaaatctctgaatctagagccattgcatgctcttgcctgttgcctaattgtagtgctccgagcatcagattcatgtcagcatcaaaacttgcttcttatttcttatcgtcgactcatccttgatcaatgtggccaacaaagatttgtgagcgctaagttgcatccacgtgttgatcatgcatataaacgcaaatgggtcattttctggaatcaagaggatttggccaactcgcttttcgttgtcacaaggtctactactagggtctcatccaaaagattcaacctaagaagatttgatagcaatgtgctgtcgctgttatgttaagattgttaggatcacaatctgtttacagcattacatcctgacagccattctcagtgggactggaagtacaaaacgtggtgttcagaacagtaattttcaaggtagagattgctgatatatatgagaataatttcttggctatcatattaatgttaccaacacaaggtttgtaccttaatcttcatagatttttcatggtgactcgctcatgctagtcatgacttgatgaatatgcaaggagcagtcttcagggatgttactgtcagacagggccaggcatctgaagaccatctgtctaagtgacaggaagtcttcaggcttcagagaacagtcaagattcacttaattaagatggcctgtggctgatctaggtagtcattagtcaaccaaatttcttcatgttccttttcttttccttcctatcttacactaatatagtaacatccagacagtcacgtatcctcctacctttgtgttatggtgagactaactgtgttctggaaggtgtgaaatccctcaccaaaatggctgaagaattgagaattcagaagccatggcagaagtgatcatgtgcatgatgaattgatgataatatatcagggggccctcatctggtcatctcacctgcctctctcttttctctttttctgagacccaaatcttgcataagacttctgtgattagacaggaatcttgtatcctttccccctatggaaagaagcctccattttgtgatatatggctcacatttttattcctgatcaggggcaagatcacaaaaaggtgcttcactgttgacccatcactaccacttttgtggatttgcttgatggcgtgatgcataatttctctatagtcaaaagtcaagcatattttgatagtggttgagaaagtaccgtgaggtaaagtaccttatgctatatcacaagtccataacaccggaaacatataggatgagtttttttcttaactttcccaactcacatctctcgtgttacccgcgcacgtattttaaactgctaaacgatatattttttgcaaaagttttctatacgaaagttgctttaaaaaatcatattaatccatttttcaaaaaaaaagctaatacttaattaatcatacgttaatgagttgctctattttacgtgcatcaaggattagttcccaactgtgtatgccgaacacagccatagttctcaagacacgtaaaaaacataataaacataataattttttgagaatctctaccttcttgaataatctaaattattgcctataattcagcagccaaacgctaaaaaacttagacttttcagatcctcagaagtttgctactcaccatctacttcatacaatctcgagctctcttaaacagggcctcaaggataattttgcctccaaagcctcanaaaaagatacccaaatcctcctcatggcgaccttttgtcaactcttggaacagagaaaatggtcaggtcgtttgtcacacgatcaaacaaagtagagagaaagaaaaaagaaggaaagaaaggatgggattgggttgtttttcccctggacagaaaaagaacagggcccagcccaactaccacgacggcacgacctgaatttgtggttagctgtagatgttttcatggcacaccttccacgtgcaaacttatatatatatatatatatatatatatatatatagagagagagagagagagagagagagagagagagagagagtacttgccaccagcagcttagtgtaattatatgctcgaataataaactgaagaaaaagtgaacaagtggttggtgctgtgtaacacagtattagtgttctttggttgaagattgaaggaagatttagctcgcttttcatgtgcatattttccaaactattaaacggtcttttctaaaaaatatttatatataaagtcgctttaataaaaccatacaagtccatttttcaaatctaaaatgattaatactttattaatcgtatattaatggctaatctcgttttgcgtatctccccaatctttttatttcctttcaaacactacgtcaacttgtattttgtttttccttatttagatggataaacatgtactatatactacaatcccctgttgtcaactggtttcatttgatcattggaggacaatgtaaagaaagtactactttcttcagtcatctttatttatcttcgggatagctaattttaggggggagggggggggggggttggagaaaattcaaaggaaattttataattcttaggaatattttcttattagctcctttggagaaaaggaatacgactgacaaatatcacatgaatttagttctgatcactacaacaaaaatgctttgtagagacatttttctagtactatagatacacttttcaaatgcctttacaatactatagaggcattttaaaaaatgcctaataagtgccttacggtgaattgtctctacaaacgaagaggcattttacaaaatgtctaaaagatggtagaggcattttatagagacattaaattgtgtcacaaccatatgaaaccaatgtaaaaaaaataaaatattttcccttgtttttgacaatccttgaactcatgatcaattgcacaattcattcttatcttcaaggcactaaccaactcaaccctaagtcattacttatatgttgttgtcttgagttatttatatttagtcatttattacatacttttattctaagaagtgcctttacagagtttaaagtgtctcaagaaaatgcctttacatatcaggcacagtttaaagtgccgaaagaatgcctctacaatataaaatctaataaaatatgctgaaaatatttctaaagtgtctgtagagtaaaagttttctaggcattttttaaaatgcctctataaaatgtctctacactataaaactcctgatctaagaggcaatttgcaaaacgcctctacaaaagtgtctttatataaggtttttgttgtagtggatgcctcagttctacaggaatataagtataaacttagacctcatatttttatttttctttgagaagtccgatgcattccctccccttttctctctagtatttttcctcaaaataacttcctccaaaatccctctgaaattccaatgttttatttcctacggacaatccaaatgcataaactcttgaattcgcatgttttaaaatcacttaggaatccaaagtatatatatgacatgatattcatacattctttttctatttatgcgttttgaaaacactatattccaaagagaaacccttagctctcccgacgtcaaataagagtgaccgttctcgcattcactccatcgcactacttcatgccgcaaaatgtttccatttgaaattattgtttatttatacatacgacccacgcccgactcaactattgcatagacactactgttattttcctagacccacatagagataaactcagtgcaaggattagtggatagagatgcgatcgaatgttagtcgtacgtcatggtcgtatatagtaggccgtcatgacattagtggaacgtatggcaccctcaatatatattttttctatgaaagctgtcctctttgggagcccgatgtgaaggaaaaaatatcatgctagctttctttctgacccattcctctccctcctcctactccactcccgtagcttgtgtcgcatgaggtggagctcatttggttggcaagggagacgtcgaccggactttgtcctcggaactaggattctctttttcactaacatgtgagtccgataaatcctagacccacatggtagtgacaaaaaaaaacatggcaactttgaaggtagaggatctcaatctttgatgagcttctttctcatcctctattgtcactagagctcatttggttgggacgatgccatccattagatttggtgacatcccgagggacaaaagcggttagggggtagggaggtcagacactagagatggtacggggcaatggcgtggtggctagcgtcagggaaaataatatggagacaacaccgtacgatgacatttaccttgagccctcagatttaaggctgcgtggatttcctcgggaggacatcgtcacctcatcaccgggagcatacaagagagaagagtggatatgcgcgttgtgaattttcgatgtttcaggcagcacatacggatgtttctcgtatttcgatcaaaatgttaaagtggggattttgatggcgtttctttttttttttttgtggcacagttcctcagcaagacaagcgcacggcttcacccactcacctactacctctgcgttgtttcgccccgtctgctagcgcggcgggtcgttgtcttcttcatcaacaggaggcggcaagtagccaagtaggaggcatccccatagtcgcgcaaccttacctccggatcttcgtattatattgttttatattgtttcttcttcttcttcttcttcttcttcttcttcttcttcttgtttgtgtagcaagtagcaacggagtctcagatcagattagccgccacaggggaggggagaccatggacgaggccgccgccggccaacgcgccagtcctcttcttgccaaggtacggcgaaccgcggaaactgctaatccccgcaggcgcctatcctgaccttttcctcttgtatatatgtgtgtttcttgctttgctgccttatggggtttcaggggaagcagaatagtatgatgcaaagattgtggctttaccgatcaagatttggtttttactacagttgggtgtggggatgtgagctggagtaaatttttttgttgttgtttttttttttaaagaagaaaatgcaaatcgtagtctgaaattgagaaaagaaaaatgcgatgactgataactgctacctggatactgatctttgatgttgataaggattatgaaaccccgaagatgtctagtgcgtatttcattccgtcggtggatttggtcaaagaatgttgagttttagattgttgtaccttgttttggaacgcgaaattttgcagatcgaataagctgttttcgatctactcaatcacattgcggtgtttatgctgacgtctggccttctgtcagaatgatggatcaagctatggtgaagaatcacagagtttattggaagaacaggagccacaggttaaaactaaacaatctggctggagagcaccatcaatcattctgggtgagccttgttatataagcatacccttcttctcgtaaaatcaaaatcttctctccatcgaaaactgtgtgaaaaccaattcatatatagagggattggagcacccatcggtgcatcactatataaacattctttaccatgatgccacaactaatgctcacaaatcatgcaggacttgaatgcttggagagcatggctttcaatggcattgccacaaatctagttgtgtatattcgctcagttctccatggtggcatcgcttccagtgcttcaacttcttctctttggtacggtactagtttctttgtgcctatacttggagcaaccattgcagatacttactggggaaactataagacagtcttgatctcctttatcatgtatttacttgtaagatcagttttcctgctcaactgctcaatcttattcacatttcattgaacaattgaactactccagagtcaagatgatgcatttttgttgtagaaaggtcataatgaaataccgatgcacatttcagggtacggtattcattactgttggagcttttctgccttctgctccagccttatgcaacacggaatcatgctcatcaatgaatgggactcaacatctagtatacttctcnaggcctgtatctcactgctattggttgtggcggagtaaggtctgcgttgcttccgcttggtgcagatcaattcaacaacgatagcagtttagatatacaaaagagaaggaattcttcagtttattctacatttgtgttatctttggtgtgatacttctgg

SEQ ID NO: 6 - DNA, 593 bp, Oryza sativa
gcacgaggatcttgatggagtgggtgatggcgaggatggtgatgcacccggatgcgttccgcccggagcgcttctcggagggggaggacgtcggcgtgctcggcggcgacctccgcctcgcgccgttcggcgccggccgccgcgtctgccctggcaggatgctggcgctcgccaccgcccacctctggctcgcccagctgctgcacgccttcgactggtcccccaccgccgccggcgtcgacctgtccgagcgcctcggcatgtcgctggagatggcggcgccgctcgtgtgcaaggccgtggctagggcctgagccctagccgccgccgccgccattattgccattgatgtggctagcgacgttgtcgtgctcgcatccatactcctccataggcaactcgtctagccaatgaagaaagctactatctatctatctatcaagctagctgctactatcacaaaccgcatttcggcatcatcttaaattagctcttaggggtgtaggcgattttggtttcccccaaaaatttgctttgccagttttttggtttaaatcgaggcattagttgtgaaaaaaaaaaaaaaaaaaaaa

SEQ ID NO: 7 - protein, 100 aa, Oryza sativa
LMEWVMARMVMHPDAFRPERFSEGEDVGVLGGDLRLAPFGAGRRVCPGRMLALATAHLWLAQLLHAFDWSPTAAGVDLSERLGMSLEMAAPLVCKAVARA

SEQ ID NO: 8 - DNA, 1131 bp, Oryza sativa
gcacgagctttcgagggacggacacggtggcggtcctgatcgagtgggtggcggcgaggctggtgctgcaccaggacgtgcaggccagggtccatgacgagctggaccgagtggtcgggtcggaccgggcagtgaccgagtcggacgcgtccaagctggtctacctccaagcggtgatcaaagaggtcctgcgcctccacccgccgggcccactgctctcgtgggcacgcctcgccacgtcggatgtacacgtcggcgggttcctcataccctctgggaccaccgccatggtgaacatgtgggccataacccatgaccctgccgtttggcccgacccgaacgagttcaaaccagagaggttcgtcgcagggccctcgtcggaccaggccacggagtttccgataatggggtcggatctcaggctcgcgccgttcgggtcaggaaggcgaagctgccccggcaagtcgctcgccatcgccactgtcggattctgggttgccacgttgctacacgagttcgattggcttcccttgtcagataagtcgcgcggcgtcgatctgtcggaggtgctgaagctgtcgtgcgagatggcaaccccgctggaggcaaggctaaggccgcgacgcaaggtgtgatgacgtgtcaccaccgtcacgtgggactaagacgaggagagggaagccgacttccacttccttctagtgcttgttgagatgtgtaaatgtccctaaatgtaaagtgttacgctttgagtagaaatgcccctacgttgtagtgcgtagtattgtacacttgtagtatgtaatgcttgtatttttgtgtgttttgcacgtcctaagtagtggagtagtagctgataatagttagttaattactctgctatttagtcatagttaactacctacctgcaggtgatgagagtgacagtttttttttgtttaattaactgcaggtgatgagtgtagaatagctcggtatgcccatctctatcctaagtgcacgcgtgcgtgtgtaattattgtcagatgtatgttgttttcaatgatagtgtacatatttttggcgagctcgatcttccattaggaagtgatcgctgcatgcttacctcaaaaaaaaaaaaaaaaaaaaaaa

SEQ ID NO: 9 - protein, 208 aa, Oryza sativa
FRGTDTVAVLIEWVAARLVLHQDVQARVHDELDRVVGSDRAVTESDASKLVYLQAVIKEVLRLHPPGPLLSWARLATSDVHVGGFLIPSGTTAMVNMWAITHDPAVWPDPNEFKPERFVAGPSSDQATEFPIMGSDLRLAPFGSGRRSCPGKSLAIATVGFWVATLLHEFDWLPLSDKSRGVDLSEVLKLSCEMATPLEARLRPRRKV

SEQ ID NO: 10 - DNA, 610 bp, Oryza sativa
cttctccggagcttcaggtgggtcccgtccggcgaccgcggcgtcgacatgagcgagcgcctcggcatgtccctcgaaatggagaagccattgatctgcctcgcgcttccaaggacctcgtctacctagctacacacacaagctgctaccaactttgctaagacctctacttggaatcttgtagattatatctgttaattatgtataattaagcttccgtaaaaaaatatatgtactccctttgtttcacaatataagtcattctagcattttccacattcatattaatgctaatgattcattagcattaatatgaatgtgaaaaatactagaatgacttacattatgaaacggaggaagtataataattaagcatacgcatgttctaacctatagatcaattttcatgtgggtgcttggttagaacttgaaataatcccaaggttttgtagcctgttctttatataggggttttttttttcatgctctcgtgatgcaagtatggggtgtggtttgttctctgggagacatgagacgctaataagatgattattgtacttttttaaaaaaatggctgtggaccatatgtcataaaaaaaaaaaaaaaaaa

SEQ ID NO: 11 - protein, 42 aa, Oryza sativa
LLRSFRWVPSGDRGVDMSERLGMSLEMEKPLICLALPRTSST

SEQ ID NO: 12 - DNA, 1146 bp, Zea mays
gcacgagcgacctgctcggcatgttcaactggggtgaccacctgccgctgctcaggtggctggacctgcagggcgtcaggaggcggtgcaggagcctggtgggcagagtcaacgtgttcgtggccaggatcatcgaagagcacaggcacaagaaggacgacgccattggagagccggccgccgccggagacttcgtcgacgtcttgctgggactggatggcgaggagaagctgtcggactccgacatgatcgctgtcctctgggagatgatctttcgagggaccgacacggtggcgatcctgctggagtgggtgatggcgcggatggtgctgcacccgggcatccagtccaaggcgcaggcggagctggacgccgtggtgggccgcggccgcgccgtttgcgacgccgacgtggcccgcctgccctacctgcagcgcgtcgtgaaggagacgctccgcgtgcacccgccgggtccgctgctctcgtgggcgcgcctggccgtgcgcgacgcggtggtcggcggccacgtggtccccgcgggcaccacggccatggtcaacatgtgggccatcgcgcacgaccccgcggtgtggccggagccctccgcgttccggcccgagcggttcgaggaggaggacgtgagcgtgctgggcggcgacctccgcctcgcgcccttcggcgccggccggcgcgtgtgccccggcaagacgttggcgctcgccaccgtccacctttggctcgcgcagctgctgcaccgcttccggtgggcgccggccgacggccgcggcgtcgacctggcggagcgcctcggcatgtccctggagatggagaagcccctcgtgtgcaagcccacgccgaggtggtgaatggcgatcgctagagcgaaagcgcaactacgctacgcatggcgcgccatcgagttccatgcaaaactatattattatactactattactagcgtttcatattttgcacttgtggttttgtttacgttaattaccgttcgcgatcgatggaactgagtgaagtgtgcacagcatactccattgctagaaagaggacgagatatgtgaaaacgcctgatggctgatggcaaattatatggagagcatgtttcagtaaaaaaaaaaaaaaaaaaaaaaaaaa

SEQ ID NO: 13 - protein, 285 aa, Zea mays
DLLGMFNWGDHLPLLRWLDLQGVRRRCRSLVGRVNVFVARIIEEHRHKKDDAIGEPAAAGDFVDVLLGLDGEEKLSDSDMIAVLWEMIFRGTDTVAILLEWVMARMVLHPGIQSKAQAELDAVVGRGRAVCDADVARLPYLQRVVKETLRVHPPGPLLSWARLAVRDAVVGGHVVPAGTTAMVNMWAIAHDPAVWPEPSAFRPERFEEEDVSVLGGDLRLAPFGAGRRVCPGKTLALATVHLWLAQLLHRFRWAPADGRGVDLAERLGMSLEMEKPLVCKPTPRW

SEQ ID NO: 14 - DNA, 778 bp, Zea mays
gcgaaggcccaggcggagctggacggcgtcgtgggcatcgggcgcggcgtggcggacgccgacgtcgccagcctaccctacatccagtgcatcgtgaaggagacgctgcgcatgcacccgccaggcccgctcctgtcgtgggcgcgcctcgccgtccacgacgcgcacgtcggaggccacctggtccccgccggcaccacagccatggtcaacatgtggtccatcgcgcacgaccccgccatctgggccgagccggagaagttccgccccgagcggttccaggaggaggacgtgagcgtcctcgggagcgacctccgcctggcccccttcggcgccgggcgccgcgcctgccccggcaagatactggccctcgccaccacccacctctgggtcgcccagcttctgcacaagttcgagtgggccgccggcgggggcgtcgacctgtcggagcgcctgagcatgtcgctggagatggccacgccgctggtgtgcaaggccgtacccagggttcagggccaagcggcctcctagcctagcctccatgcatgcctgatgcctggatgccgtagcgagagtgggagactgatgagtgtatgccgttatgtttgtgtgtccatgcatgcatgcatgcctcggctactgtagcttttggcttgcttgttgtgcatgtcctgcgtcgagaccttgcgtagtatgatgcagtataattttaataataatattattattaaaggttaaagttttgataatacagtaaaaaaaaaaaaaaaaaaaaaaa

SEQ ID NO: 15 - protein, 177 aa, Zea mays
PAKAQAELDGVVGIGRGVADADVASLPYIQCIVKETLRMHPPGPLLSWARLAVHDAHVGGHLVPAGTTAMVNMWSIAHDPAIWAEPEKFRPERFQEEDVSVLGSDLRLAPFGAGRRACPGKILALATTHLWVAQLLHKFEWAAGGGVDLSERLSMSLEMATPLVCKAVPRVQGQAAS

SEQ ID NO: 16 - DNA, 1597 bp, Zea mays
ccacgcgtccggcgcaccgcaccctggcggcgctgtcccacgccgtagacggcggcaaggcactgatggccttctcggtcgggctgacccgtctcgtcgtgtcgagccagcccgatacggcgcgcgagatcctcgccagccccgcgttcggcgaccgccccatcaaggacgcggcgcgccacctgctcttccaccacgccatgggcttcgcgccctccggagacgcgcactggcgcgggctccgccgcctcgccgccaaccacctgttcggcccgcgccgcgtggcgggtgccgcgcaccaccgcgcctccatcggcgaggccatggtcgccgacgtcgccgctgccatggcgcgccacggcgaggtccctctcaagcgcgtgctgcatgtcgcgtctctcaaccacgtcatggccaccgtgtttggcaagcgctacgacatgggcagccgagagggcgccgttctggacgagatggtggccgagggctacgacctcctgggcacgttcaactgggctgaccacctgccattgctcaagcatctcgacccccagggcgtgcgccgccggtgcaataggctggtccaaaaggtcgaatcgttcgttggcaagatcatcatggagcacaggacgaggcgcgcaaatggaggagtcgtgggcgatgagtgcatgggtgacttcgtcgacgtccttcttggcctcgagggagaggagaagctgtcagatgaggacatgatcgctgttctttgggagatgatcttcagaggcgccgacaccgtggcgatcttgatggagtgggtcatggcgaggatggcgctgcacccggacatccaggcgaaggcccaggcggagctggacggcgtcgtgggcatcgggcgcggcgtggcggacgccgacgtcgccagcctaccctacatccagtgcatcgtgaaggagacgctgcgcatgcacccgccaggcccgctcctgtcgtgggcgcgcctcgccgtccacgacgcgcacgtcggaggccacctggtccccgccggcaccacagccatggtcaacatgtggtccatcgcgcacgaccccgccatctgggccgagccggagaagttccgccccgagcggttccaggaggaggacgtgagcgtcctcgggagcgacctccgcctggcccccttcggggccgggcgccgcgcctgccccggcaagatactggccctcgccaccacccacctctgggtcgcccagcttctgcacaagttcgagtgggccgccggcgggggcgtcgacctgtcggagcgcctgagcatgtcgctggagatggccacgccgctggtgtgcaaggccgtacccagggttcagggccaagcggcctcctagcctagcctccatgcatgcctgatgcctggatgccgtagcgagagtgggagactgatgagtgtatgccgttatgtttgtgtgtccatgcatgcatgcatgcctcggctactgtagcttctggcttgcttgttgtgcatgtcctgcgtcgagaccttgcgtagtatgatgcagtataattttaataataatattattattaaaggttaaaaaaaaaaaaaaaaaaaaaaaaaaaa

SEQ ID NO: 17 - protein, 451 aa, Zea mays
PAHRTLAALSHAVDGGKALMAFSVGLTRLVVSSQPDTAREILASPAFGDRPIKDAARHLLFHHAMGFAPSGDAHWRGLRRLAANHLFGPRRVAGAAHHRASIGEAMVADVAAAMARHGEVPLKRVLHVASLNHVMATVFGKRYDMGSREGAVLDEMVAEGYDLLGTFNWADHLPLLKHLDPQGVRRRCNRLVQKVESFVGKIIMEHRTRRANGGVVGDECMGDFVDVLLGLEGEEKLSDEDMIAVLWEMIFRGADTVAILMEWVMARMALHPDIQAKAQAELDGVVGIGRGVADADVASLPYIQCIVKETLRMHPPGPLLSWARLAVHDAHVGGHLVPAGTTAMVNMWSIAHDPAIWAEPEKFRPERFQEEDVSVLGSDLRLAPFGAGRRACPGKILALATTHLWVAQLLHKFEWAAGGGVDLSERLSMSLEMATPLVCKAVPRVQGQAAS

SEQ ID NO: 18 - DNA, 1539 bp, Zea mays
  unsure (348): n = A, C, G, or T
gcgctgcgccgcgtggcgtccacgcacctcttctccccgcggcaggtcgccgcgtcggccgcgcagcgcgccgtcatcgcgcgccagatggtcggcgccgtcaaggagctgtcggcggcctcgccggggcggcgcggcggcgtcgaggtccgccgcgtcctgcgccgcggctccctgcacagcgtcatgtggtcggtgttcggccggcggtacgacctggagctggacccggccagggagagccccgagacgcgggagctgaggcgactcgtggacgaagggtacgacctgctgggccagatcaactggtccgaccacctccccggcctcgcgtgcctcgacctgcanagcaccagggccaggtgcgaccgcctcgtcccgctcgtgacccgcttcgtcggcggcatcgtcgacgagcaccgcgcccggaaccacctccgctctgctccgcctgccgtcgtggacttcaccgacgtcctgctctcgctgccggccgacgacaggctcaccgacgctgacatgatcgccgtcctctgggaaatggtgttccgtggaactgacaccgtcgccgtgctgatggagtgggcgctggccaggctcgtgctgcaccctgacgtgcaggcccgcgtccacgacgagctggaccgcgtggtcgggcccgaccgggccgtcaccgagtccgacacggcgtcactggtctacctgcacgccgtgatcaaggaggtgctcaggatgcacccgccgggcccgctgctgtcgtgggcgcgcttggccacgtcagacgtgcacgtcgacgggcacctcatccccgccgggaccaccgcgatggtgaacatgtgggccattacgcacgacccagacgtgtgggccgagccgacggagttccagccggagaggttcatggggtccaccgagttcccgatcatggggtcggacctcaggctcgcgccgttcggggcgggccggcgcagctgccccgggaagagcctcgccatggccaccgtggccttctggctcgcgacgctgctgcacgagttcgagctgctcccctcgcccgtcgacctgtcggaggtgctcaagctgtcgtgcgagatggccgtcccgctggcgctggccgtgacggcgaggccccggcaagcggttcagaagtcggttggggtatcagtctcactgtgagcaatagcatggcgggctggcgctactgtacatggaaagtgcttctgcttgcaggttgctactactcggtcgacatgggtatatgcttttcatgttactgtctttgatgtgtatcgatcaggtgccgaatgtgatactttggcttgtactgttagctcttttcctgggtgctcttttctttctttttcttagtactcgctgtaagactcgtcaaatgtatatgctggtttggatggttttggattgtagtcgcatactactagtagtattgcgcagttcaatgcctaaatatgctataatcaaaaaaaaaaaaaaaaaaaaaaaaaaaaaaaaaaaaaaaaaaaaaaaaaaaaaaaaaaaaaa

SEQ ID NO: 19 - protein, 391 aa, Zea mays
  UNSURE (116): Xaa = any amino acid
ALRRVASTHLFSPRQVAASAAQRAVIARQMVGAVKELSAASPGRRGGVEVRRVLRRGSLHSVMWSVFGRRYDLELDPARESPETRELRRLVDEGYDLLGQINWSDHLPGLACLDLXSTRARCDRLVPLVTRFVGGIVDEHRARNHLRSAPPAVVDFTDVLLSLPADDRLTDADMIAVLWEMVFRGTDTVAVLMEWALARLVLHPDVQARVHDELDRVVGPDRAVTESDTASLVYLHAVIKEVLRMHPPGPLLSWARLATSDVHVDGHLIPAGTTAMVNMWAITHDPDVWAEPTEFQPERFMGSTEFPIMGSDLRLAPFGAGRRSCPGKSLAMATVAFWLATLLHEFELLPSPVDLSEVLKLSCEMAVPLALAVTARPRQAVQKSVGVSVSL

SEQ ID NO: 20 - DNA, 1764 bp, Glycine max
gcacgaggtcccttcttcctctatctctttggctattagcaaacactctcatatttggttgttctagttctcactaccatgtcaacccacattgaaagcctgtgggtgttggccttagcctcaaaatgcattcaagagaacattgcatggtcactcttgatcatcatggtcactctctggctcaccatgaccttcttctactggtctcaccctggtggtcctgcttggggcaaatactactactttaattactggaaaaaaaccacctcaaccaacacaaacatcaaccttaaaatgattatccctggtcctagaggctaccctttcattgggagtatgagtctcatgacatccctcgcacaccaccgtattgctgcggcgggggaagcatgcaacgccaccaggctcatggctttttccatgggtgacacacgcgccatagtaacgtgcaaccccgatgtcgctaaagagattctcaatagttccacttttgctgatcgtcccataaaggaatcagcttacagcctcatgttcaaccgcgccatcggcttcgccccttacggcgtctactggcgtaccctccgccgcatcgccgccacgcacctcttctgccccaaacaaatcaaagcctccgagctccagcgcgctgaaatcgccgcccaaatgacaaactcattccgaaatcaccgttgcagcggcggtttcggaatccgcagcgtgctcaagagagcgtcactgaacaacatgatgtggtcggtgtttggacaaaagtacaaccttgacgagataaacaccgcaatggacgagctatccatgttggtggaacaaggctatgacttgttgggcacccttaattggggagaccatatccctttcctgaaagactttgacctacagaaaatccggttcacctgctccaaattagtccctcaagtgaaccggttcgttggttcaatcatcgccgaccaccaggccgacacaacccaaaccaaccgcgatttcgttcatgttttgctctctctccaaggtcccgataaattgtctcactccgacatgattgctgtcctctgggaaatgatatttagggggaccgacacggtggcggttttgattgagtggatactggcgaggatggtgcttcatccggaggtgcaaaggaaggtacaagaggagttggacgcggtggttaggggtggcgctttgacggaggaggtcgtggcggcgacggcgtatcttgcggcggtggtgaaagaggttctgaggctgcacccgccgggcccgcttctctcgtgggcccgcttggccatcactgatacgaccattgatgggtatcacgtgcctgcggggaccaccgctatggttaatatgtgggccatagcaagggacccggaggtgtggctggacccacttgagttcaagcccgagaggttcatgggtctggaaaacgagttttctgttttcgggtcggatctgagactcgctccattcggttcgggtcggagaacatgccccgggaagactttgggtttgagcaccgtaaccttctgggtggcttggcttttgcatgagtttgaatggctaccgtctgatgaagccaaggttgatctaacggaggtgctgaggctctcgtgtgaaatggctaacccactcattgttaaagttcgccctaggcatggattaagcacttaatgataatataattaagcctatctacgttattaacttgaaatgttttaatgggaaggaaaaaaaaaaaaaaaaaaa

SEQ ID NO: 21 - protein, 536 aa, Glycine max
MSTHIESLWVLALASKCIQENIAWSLLIIMVTLWLTMTFFYWSHPGGPAWGKYYYFNYWKKTTSTNTNINLKMIIPGPRGYPFIGSMSLMTSLAHHRIAAAGEACNATRLMAFSMGDTRAIVTCNPDVAKEILNSSTFADRPIKESAYSLMFNRAIGFAPYGVYWRTLRRIAATHLFCPKQIKASELQRAEIAAQMTNSFRNHRCSGGFGIRSVLKRASLNNMMWSVFGQKYNLDEINTAMDELSMLVEQGYDLLGTLNWGDHIPFLKDFDLQKIRFTCSKLVPQVNRFVGSIIADHQADTTQTNRDFVHVLLSLQGPDKLSHSDMIAVLWEMIFRGTDTVAVLIEWILARMVLHPEVQRKVQEELDAVVRGGALTEEVVAATAYLAAVVKEVLRLHPPGPLLSWARLAITDTTIDGYHVPAGTTAMVNMWAIARDPEVWLDPLEFKPERFMGLENEFSVFGSDLRLAPFGSGRRTCPGKTLGLSTVTFWVAWLLHEFEWLPSDEAKVDLTEVLRLSCEMANPLIVKVRPRHGLST

SEQ ID NO: 22 - DNA, 1934 bp, Glycine max
ctcttcttagttccagcacaacaagctcttcatttctcccacactttcttttctttcaccaaaaatgtcaccagatttcacacttttgttcttcccggaactcatgcagtcccctatgatcactttccaagccaccctctgcgtccttctcttcaccctcatgttcacgctgctcttcactcctggtgggcttccttgggcctgggcccggcccagacccatcatccctggcccagtaactgccctgttagggatctttactggctccacgcctcaccgtgctttatccaaactcgcccgtaattaccacgcggaaaaactcatggctttctccatcggtttaacccgtttcgtcatctccagcgaaccggagaccgctaaggagattctcggcagccccagtttcgctgataggccggtgaaggaatccgcctatgagcttctcttccaccgcgcaatgggttttgcaccgtatggggagtactggaggaatttgaggagaatctcagccctacatctcttctccccgaagagaatcaccggctctgaatccttcaggagcgaggttggattaaaaatggttgaacaagttaagaaaaccatgagtgagaaccaacatgttgaggttaagaaaattctacactttagttcgttgaacaatgtgatgatgacggtgtttggtaagtcttatgagttttacgagggtgagggtttggagcttgagggtttggtgagtgaagggtatgagttgttgggtgtttttaactggagtgaccattttccggttttggggtggttggatttgcagggtgtgaggaagaggtgtaggtgtttggttgaaaaggttaatgtttttgttggaggggttattaaggagcatagggtgaagagggagaggggtgagtgtgtgaaggatgaaggaactggggattttgttgatgttttgcttgatttggagaaggaaaacaggctcagtgaagctgacatgatcgctgttctttgggaaatgatatttaggggaactgacacggtggcaattctgctagagtggactctggctcggatggttctccaccctgaaatccaagcaaaggcacagcgcgaaatagacttcgtttgcggatcctccaggcccgtatccgaagcagacattccgaacctgcgctaccttcagtgcatagtaaaagaaacccttcgtgtgcacccaccaggcccgctactctcgtgggctcgccttgctgtgcacgacgttaccgtgggcggcaagcacgtgattcccaagggcaccaccgcgatggtgaacatgtgggccataacccacgacgagagggtgtgggccgagcccgagaagtttaggcccgagcggtttgtggaggaggatgtgagcataatggggtctgatttgaggttggcacctttcgggtctggaagaagagtgtgccctgggaaggcccttggtttggcctcggttcatctttggctcgctcagttgcttcaaaattttcattgggtttcatctgatggtgtttctgtggagttggatgagtttcttaagctttctatggagatgaagaagccactgtcttgcaaggctgtgcctagggtttctgtttaggtttatgtgtgttgttgggttgagttggtttggtttgtctgcttaggtttgtggatgttgttcccaaggctgtgcctagggtttctgtttaggtttatgtgtgttgtttggtttgtctgtttaggtttatggatgttgtttggttgagttggtttggtttgtgttatctgctaagtttagttcaagaaaagtagggtttagagcacctttttattaatcgctaggggttgttattccgtgtacggtttgtagtaagttgtaaaagactagaagagaatgtaagaggttttgttttgtgtgggtcgttaaaaaaaaaaaa

SEQ ID NO: 23 - protein, 516 aa, Glycine max
MSPDFTLLFFPELMQSPMITFQATLCVLLFTLMFTLLFTPGGLPWAWARPRPIIPGPVTALLGIFTGSTPHRALSKLARNYHAEKLMAFSIGLTRFVISSEPETAKEILGSPSFADRPVKESAYELLFHRAMGFAPYGEYWRNLRRISALHLFSPKRITGSESFRSEVGLKMVEQVKKTMSENQHVEVKKILHFSSLNNVMMTVFGKSYEFYEGEGLELEGLVSEGYELLGVFNWSDHFPVLGWLDLQGVRKRCRCLVEKVNVFVGGVIKEHRVKRERGECVKDEGTGDFVDVLLDLEKENRLSEADMIAVLWEMIFRGTDTVAILLEWTLARMVLHPEIQAKAQREIDFVCGSSRPVSEADIPNLRYLQCIVKETLRVHPPGPLLSWARLAVHDVTVGGKHVIPKGTTAMVNMWAITHDERVWAEPEKFRPERFVEEDVSIMGSDLRLAPFGSGRRVCPGKALGLASVHLWLAQLLQNFHWVSSDGVSVELDEFLKLSMEMKKPLSCKAVPRVSV

SEQ ID NO: 24 - DNA, 1905 bp, Glycine max
gcacgagcttcctctttctctctttaaatacacacacacacacactcactttcttgcttgttctaactaccatgacaacccacattgataacctgtgggtgttggccttggtctcaaaatgcacacaagagaacattgcatggtcactcttgaccatcatggtcactctctggctctccatgaccttcttctgctggtctcatcccggtggtcctgcttggggcaagtactactcctttcattactggaaaaaaacaaccacaaccacaacctcaacctcaaacaacacaaactccaacaaccttaaaatgattcccggtcccaaaggctatcctttcattggaagcatgagcctcatgacatcccttgcacaccaccgtattgctgccgctgctcaagcatgcaaagccaccaggctcatggccttctccatgggtgacacgcgtgtcatcgtcacgtgccacccccacgtggccaaggagattcttaacagctccgtcttcgccgatcgtcccataaaggaatcagcctacagcctcatgttcaaccgcgccatcggctttgccccttacggcgtttactggcgcaccctccgccgcatcgccgccacgcacctcttctgccccaaacaaatcaaggcctcggagctccagcgcgccgaaatcgccgcccagatgacccactcgttccgaaaccgccgcggcggtttcggaatccgcagcgttctcaagagagcgtcgctcaacaacatgatgtggtcggtgtttggacaaagatatgaccttgacgagacaaacacttcagtggacgagttatcccggttagtggaacaaggctatgacttgttgggtacccttaattggggagaccatatcccttttctgaaagactttgaccttcaaaaaatccggtttacctgctccaaactcgtcccccaagtgaaccggttcgtaggttcaatcatcgccgaccaccaaaccgacacaacccaaaccaaccgcgatttcgttcatgttttgctctctctccaaggtcccgataaattgtctcactccgacatgattgctgtcctctgggaaatgatatttagggggaccgacacggtggcggttttgattgagtggattatggcaaggatggtgcttcatccggaggtacaaaggagggtgcaagaggagctggacgcggtggttggaggtggtgcgcgcgctttgaaggaggaggacgtggcggcgacggcgtatcttctggcggtggtgaaggaggttctgaggctgcaccctccaggcccgcttctctcgtgggcccgcttggccatcaccgatacgaccattgatgggtataacgtgcccgcgggaaccaccgccatggttaatatgtgggccataggaagggacccggaggtgtggctggacccacttgatttcaagcccgagaggttcatgggcctggaggcggagttttctgttctcgggtcggatctgaggctggctccattcgggtcgggtagaagaacctgccccggaaagactttgggtttgagcaccgtgactttctgggtggcgaggcttttgcacgagtttgaatggctaccatctgatgaggggaaggttgatctaacggaggtgctgaggctctcgtgtgaaatggctaacccgctctatgttaaagttcgccctaggcgtggattaagtacttaataataataataataataataataataataataataatgttaagtagcaggtgcatggccctttggagccactaaatgttaagtgaatccatgaatcaaggtagaaagtttgagttggctctgtctctataatatgggtcaacgggtttttgtttaaaaaaaaaaaaaaaaaaaaaaaaaaaaaaaaaaaaaaaaaaaaaaaaaaa

SEQ ID NO: 25 - protein, 542 aa, Glycine max
MTTHIDNLWVLALVSKCTQENIAWSLLTIMVTLWLSMTFFCWSHPGGPAWGKYYSFHYWKKTTTTTTSTSNNTNSNNLKMIPGPKGYPFIGSMSLMTSLAHHRIAAAAQACKATRLMAFSMGDTRVIVTCHPHVAKEILNSSVFADRPIKESAYSLMFNRAIGFAPYGVYWRTLRRIAATHLFCPKQIKASELQRAEIAAQMTHSFRNRRGGFGIRSVLKRASLNNMMWSVFGQRYDLDETNTSVDELSRLVEQGYDLLGTLNWGDHIPFLKDFDLQKIRFTCSKLVPQVNRFVGSIIADHQTDTTQTNRDFVHVLLSLQGPDKLSHSDMIAVLWEMIFRGTDTVAVLIEWIMARMVLHPEVQRRVQEELDAVVGGGARALKEEDVAATAYLLAVVKEVLRLHPPGPLLSWARLAITDTTIDGYNVPAGTTAMVNMWAIGRDPEVWLDPLDFKPERFMGLEAEFSVLGSDLRLAPFGSGRRTCPGKTLGLSTVTFWVARLLHEFEWLPSDEGKVDLTEVLRLSCEMANPLYVKVRPRRGLST

SEQ ID NO: 26 - DNA, 2924 bp, Glycine max
gcacgagaaaaaagctcatgacattgagtctaggaacaaatccagttgttatcagcagtcacccagaaaccgcaagagaaattctttgtgggtcgaacttcgctgaccgacccgttaaagaatcggcccgaatgctcatgtttgagcgtgccattggatttgctccatatgggacttattggcgccacctacgtaaagtggcaatcacccacatgttctctccaaggaggatttctgacttggagagtctccgacaacatgtggttggtgaaatggtgatgaggatatggaaggagatgggggacaaaggggtggtagaggttcgaggcatattgtatgaagggtctttgagccacatgttggagtgtgtgtttggtattaataattctctaggatcacaaacaaaggaggcgttgggtgatatggttgaggaagggtatgacttgattgccaagtttaattgggcagactattttcctttcgggtttttggactttcacggggtcaagagaaggtgtcacaaattggcaactaaggtcaatagtgtggtgggtaaaattgtggaagaaagaaaaaattcagggaagtacgttggacaaaatgattttcttagtgccttgttattgttgcctaaagaggaaagcataggtgattcagatgtagtggctatcttatgggaaatgatatttcggggaacagacacaattgctatacttttagaatggatcatggccatgatggttttacaccaagacgtacaaatgaaagctcgtcaagagatcgactcatgcatcaagcaaaacggttacatgcgagactcagacattccaaacctcccttacctccaggccatagtgaaggaggttctccgattgcacccaccaggcccattactttcctgggctcgcctcgcaatccatgatgtccacgtggacaaggtcatcgtgccagctggcacaactgcaatggttaacatgtgggctatatcacatgactcatccatttgggaggacccgtgggcctttaagcccgaaagattcatgaaagaagatgtgtcgatcatggggtcggacatgagacttgcaccatttggtgcaggacgtagggtgtgcccaggaaaaacattaggcttagccacagttcatctatggcttgcacaacttcttcaccatttcatatggattccagtgcaacccgtggatctttcagaatgcctaaagctctcgctcgaaatgaaaaagcctttacgatgccaagtgattcgcaggttcaacaccataagctcttgaactcaacaagataaattaatgcacaataaaggatatcattatcgatgtaactgttgtgataaaaaaaaattaaagtctttgatttgggtggaagttatgtaatgttgtaaaaatatatcaagtactgagagatcccctcataatttccccaaagcgtaaccatgtgtgaataaattttgagctagtagggttgcagccacgagtaagtcttcccttgttattgtgtagccagaatgccgcaaaacttccatgcctaagcgaactgttgagagtacgtttcgatttctgactgtgttagcctggaagtgcttgtcccaaccttgtttctgagcatgaacgcccgcaagccaacatgttagttgaagcatcagggcgattagcagcatgatatcaaaacgctctgagctgctcgttcggctatggcgtaggcctagtccgtaggcaggacttttcaagtctcggaaggtttcttcaatctgcattcgcttcgaatagatattaacaagttgtttgggtgttcgaatttcaacaggtaagttagttgctagaacccatggctcctttgccgacgctgagtagattttaggtgacgggtggtgacaatgagtccgtgtcgagcgctgattttttcggcctttagagcgagatttatacaatagaatttggcatgagattggattgcttttagtcagcctcttatagcctaaagtctttgagtgactagatgacatatcatgtaagttgctgataggtttccagttttccgctcctaggtctgcatattgtacttttcctcttactcgacttaaccagtaccaacccagcttctcaacggatttataccatggcactttaaagccagcatcactgacaatgagcggtgtggtgttactcggtagaatgctcgcaaggtcggctagaaattggtcatgagctttctttgaacattgctctgaaagcgggaacgctttctcataaagagtaacagaacgaccgtgtagtgcgactgaagctcgcaataccataagtcgtttttgctcacgaatatcagaccagtcaacaagtacaatgggcatcgtattgcccgaacagataaagctagcatgccaacggtatacagcgagtcgctctttgtggaggtgacgattacctaacaatcggtcgattcgtttgatgttatgttttgttctcgctttggttggcaggttacggccaagttcggtaagagtgagagttttacagtcaagtaatgcgtggcaagccaacgttaagctgttgagtcgttttaagtgtaattcggggcagaattggtaaagagagtcgtgtaaaatatcgagttcgcacatcttgttgtctgattattgatttttcgcgaaaccatttgatcatatgacaagatgtgtatccaccttaacttaatgatttttaccaaaatcattaggggattcatcagtatcaagtatgtagtatgcgttgagctcaagatagtccaagaaatgggctaatgaatggattgatactatctctctttgaaagtacaccacgtacaatattggatctaataaagtcgcatggtttttgtaaaaaaaaaaaaaaaaaaaaaaaaaaaaaaaaaaaaaaaaaaaaaaaaaaaaa

SEQ ID NO: 27 - protein, 423 aa, Glycine max
MTLSLGTNPVVISSHPETAREILCGSNFADRPVKESARMLMFERAIGFAPYGTYWRHLRKVAITHMFSPRRISDLESLRQHVVGEMVMRIWKEMGDKGVVEVRGILYEGSLSHMLECVFGINNSLGSQTKEALGDMVEEGYDLIAKFNWADYFPFGFLDFHGVKRRCHKLATKVNSVVGKIVEERKNSGKYVGQNDFLSALLLLPKEESIGDSDVVAILWEMIFRGTDTIAILLEWIMAMMVLHQDVQMKARQEIDSCIKQNGYMRDSDIPNLPYLQAIVKEVLRLHPPGPLLSWARLAIHDVHVDKVIVPAGTTAMVNMWAISHDSSIWEDPWAFKPERFMKEDVSIMGSDMRLAPFGAGRRVCPGKTLGLATVHLWLAQLLHHFIWIPVQPVDLSECLKLSLEMKKPLRCQVIRRFNTISS

SEQ ID NO: 28 - DNA, 528 bp, Helianthus sp.
  unsure (476): n = A, C, G, or T
gcacgagtggcattgcaaaataggtgtgtcagatatgactgatgaaggtgggaacccgatctggaagaaccgagttttgagtcaacagctccgattttgcggaccggcccattaaggaatctgcttatgaactgttgtttcaccgggctatggggtttgcaccctatggtgactactggaggagtttgaggagaatctcggcgacccatttgtttagcccgaaacgggttgctgggtttggggtgtttcgtgaaactattgggttgaaaatggtgggtcaggttgtgtccaccatggaacaaaacggtgtcgtggaggttaaaaagattcttcactttggttccttaaacaatgtcatgatgtctgtgtttggaaggttgtatgattttggtgaaaatggtggtgaggggtgtgagcttgaggaacttgtgagtgaaggttatgagttgttggggatatttaactggagtgaccantttccggttgttagttggtttgatttgcaaggtgtcnnnnngnnntgtnnnn

SEQ ID NO: 29 - protein, 144 aa, Helianthus sp.
  UNSURE (132): Xaa = any amino acid
VNSSDFADRPIKESAYELLFHRAMGFAPYGDYWRSLRRISATHLFSPKRVAGFGVFRETIGLKMVGQVVSTMEQNGVVEVKKILHFGSLNNVMMSVFGRLYDFGENGGEGCELEELVSEGYELLGIFNWSDXFPVVSWFDLQGV

SEQ ID NO: 30 - DNA, 457 bp, Helianthus sp.
  unsure (272): n = A, C, G, or T
gctatcgaaagcccgatcgaaaacaacaattcccggcccttccggtatccctatactcggtctcatatttgccttcacatcttccatgactcacagaacccttgcaaaactctctgtagcatttaatgctacacatttaatggcgttctccgtcggattgactcgctttgttatctcgagtcacccggagaccgccaaagagatcctcaacagctctgcgttcgcggaccggcccgttaaggagtccgcgtacgagctgttgtttcataaanccatggggttcgctccgtacggggaatattggcgaaaccttaggcggatatcagctattcatatgttaagcccgaaaaggggtatccgggtcccgggatttttttcggggctaaaaacaagggctgaaagtttgggtgaaatcaagattctcctaactttccaatgaaaattgtnaaagggttcc

SEQ ID NO: 31 - protein, 117 aa, Helianthus sp.
  UNSURE (91): Xaa = any amino acid
LSKARSKTTIPGPSGIPILGLIFAFTSSMTHRTLAKLSVAFNATHLMAFSVGLTRFVISSHPETAKEILNSSAFADRPVKESAYELLFHKXMGFAPYGEYWRNLRRISAIHMLSPKR

SEQ ID NO: 32 - DNA, 615 bp, Triticum aestivum
  unsure (24): n = A, C, G, or T
gggacgcgccgctcgagttccggncggagcggttcctggccggcggggaggccccggacgtgtccgtgctcggcgccgacggncggctcgtgccgttcgggtccggacggaggagctgcccgggcaagtccctggccatgaccacggtgaccgcgtggatggccaccctgctgcacgagttcgagtgggcgccggcggcgcccggcgtcgacctgtcggaggtgctccgcctgtcgtgcgagatggcggcgccgctccaggtccgggcgcgcccgaggcgcgacgcgtgatgtgctcgtcgcgccatggccggccggtcgactcgacccaccgtccctactacagtacgtagctcgtagcccgtgaccccgtgcttcacgaaagtgaataattaaagctgccggcgtaaaatgtacgtgcgccgagcgcagctcagtgttgagtttctttctaacgtgtgtgatgtctgtgctatgtaatgtaacccatcangtgtgagcgtgagagtgactgantgaggttcacatgtgtacaaaattgcanaacaaaatctataanagatttttgcggagtgtgcnatagtacacgttgggggggcccggtaccattccccta

SEQ ID NO: 33 - protein, 95 aa, Triticum aestivum
  UNSURE (8): Xaa = any amino acid
DAPLEFRXERFLAGGEAPDVSVLGADGRLVPFGSGRRSCPGKSLAMTTVTAWMATLLHEFEWAPAAPGVDLSEVLRLSCEMAAPLQVRARPRRDA

SEQ ID NO: 34 - DNA, 1930 bp, Aquilegia vulgaris
gcacgaggctctctttcacgaaaaccacctttctctttttctctctctaccttcaaaaccactaataatgtcttcagaaaaccttcttttctttctcccttcatcaagctttgaactttcactctgttttcttcttcttgtagccatttttggcttttggttagcacctggtggtttagcttgggctatttcaaagactcattctcaagttcaagctaaaaccgccattcctggaccatctgggtttcctttattgggtttggtctttgcttttactggttctactactcatagagttttagcaaatcttgctaaaacctttaaagctattcctttaatggctttttctgttggttttactcgttttatcatatcaagttgtcctgatacagcaaaagagattcttaatagttcttcttttgctgatcgacctgttaaggaatctgcttatgaacttttgtttcacagagcaatgggttttgctccttttggtgaatattggaggaatctgagaagaatctcagctacccatttattcagtccaaagagaataaccggttttgctacatttcgaagtgaaataggagaaaaaatgattaatgagattaaatgtcaaatggggttaaatggggaagttgaagttaaaagggtattacactttgggtctttaaacaatgtgatgatgagtgtttttggaacgttttatgattttaaacaacttaatggtgatgggtttaaacttgaagagttggtgagtgaagggtatgagttgcttgggatttttaactggagtgatcactttcctcttatgggctggttggatttgcaaggagtaaggaagagaagcagagtgttggtttctaaggtgaatatttttgttggaaaaattattgaagaacacagaaacagaaggattaatggtgttttgggtcaagaatgtgttggtgactttgttgatgtcttgcttgatttggagaaagaacatagtctcagtgactctgacatgattgctgttctttgggaaatgatctttaggggcacagacacagtagcaatcctcttagagtggattcttgcaagaatggccctacatccagatattcaagcaaaagcccaatctgaaattgacactgtcgttggcactaatcgactagtatctgattctgacttacccaaccttccttatctccaagcagtagtgaaggaatccttaagggtgcaccctcctggccccctcttgtcgtgggcacgactagctatccatgatgtccatattgggaagaactttatcccagctgggactactgctatggtgaatatgtgggcaatcactcatgatgaaagtatttggtctgagccaaatgaatttaaacccgagcgattcatcgatgaagatgtgagcattatggggtctgatctgaggttggcaccttttgggtctggaaggagggtttgtcctggaaaggctttgggtatggctactgtgcagctatggttgggtcagttacttcaaagtttcaaatgggttccttctgaaggtggtgtggacttgtctgagtgtcttaatctgtctctggaaatgaagaagcctttgatctgcaaggctattccaaggtttgcctgaagtttacctatgatgatggggaggagtacttggttcttaaaatttgttttgttcctctccttttagttgtgttctaggcttctagctaggatcatatggtttttacttttgtgtcttttgtgtccttaaaggtttataggtgaaagtaggattagtagtaatgccagattcaggagctaaaggttctctcttttgttgattatgatctggttggtacttttgatatgtatacattaaagttatggtgccatgcatacaacctttaatatatatactggatttctataaaaaaaaaaaaaaaaaaaa

SEQ ID NO: 35 - protein, 518 aa, Aquilegia vulgaris
MSSENLLFFLPSSSFELSLCFLLLVAIFGFWLAPGGLAWAISKTHSQVQAKTAIPGPSGFPLLGLVFAFTGSTTHRVLANLAKTFKAIPLMAFSVGFTRFIISSCPDTAKEILNSSSFADRPVKESAYELLFHRAMGFAPFGEYWRNLRRISATHLFSPKRITGFATFRSEIGEKMINEIKCQMGLNGEVEVKRVLHFGSLNNVMMSVFGTFYDFKQLNGDGFKLEELVSEGYELLGIFNWSDHFPLMGWLDLQGVRKRSRVLVSKVNIFVGKIIEEHRNRRINGVLGQECVGDFVDVLLDLEKEHSLSDSDMIAVLWEMIFRGTDTVAILLEWILARMALHPDIQAKAQSEIDTVVGTNRLVSDSDLPNLPYLQAVVKESLRVHPPGPLLSWARLAIHDVHIGKNFIPAGTTAMVNMWAITHDESIWSEPNEFKPERFIDEDVSIMGSDLRLAPFGSGRRVCPGKALGMATVQLWLGQLLQSFKWVPSEGGVDLSECLNLSLEMKKPLICKAIPRFA

SEQ ID NO: 36 - DNA, 884 bp, Vitis sp.
ggaaaaggaaagcaggctcagcgactctgatatgattgctgttttatgggaaatgatctttagagggactgacacggtggcaattctgttggagtggattcttgcaagaatggttttacaccccgatattcaatccaaagcccaatctgaaatagatgcagtggttggagccacccgactggtgtctgattcagacattcataaactcccttatctccatgccatagtaaaggaaactctccgcatgcatccacctggcccgctcctttcctgggcacgcctttccattcatgatacccacattggttcgcacttcatccctgcaggcaccacagctatggtgaatatgtgggcaataacccatgatgatgctgtgtgggatgagcctaaggaattcaagccaagtcgctttatggaggaggatgtgagcattttgggttctgatcttaggttggcaccatttggctctggaagaagggtttgtcctgggaaagcaatgggtttagcaactgtgcaactgtggttggctcaattgctccaaaacttcaaatgggttgcttgtgactctggtgtggacttgtctgagtgcctcaagctctcaatggagatgaaacagtccttggtttgcaaggctgttcctaggttctcttgaaatatgaattgatgatggggtttgacaatgatttgggtgtgatctcatccatgattttggaagccttgtatggtgaggtcaaacagattacttactatggttttccttagcgttttaatatccttgttataagaacagtaccgttgttggcttgaaaggtcgtggttgtgtaatgaaagtgcttggctctggttaggtgcgaaaaaaaaaaaaaaaaaaaaaaaaaaaaaaa

SEQ ID NO: 37 - protein, 216 aa, Vitis sp.
EKESRLSDSDMIAVLWEMIFRGTDTVAILLEWILARMVLHPDIQSKAQSEIDAVVGATRLVSDSDIHKLPYLHAIVKETLRMHPPGPLLSWARLSIHDTHIGSHFIPAGTTAMVNMWAITHDDAVWDEPKEFKPSRFMEEDVSILGSDLRLAPFGSGRRVCPGKAMGLATVQLWLAQLLQNFKWVACDSGVDLSECLKLSMEMKQSLVCKAVPRFS

SEQ ID NO: 38 - DNA, 442 bp, Parthenium argentatum Grey
  unsure (340): n = A, C, G, or T
gtcgatgttttgcttgatttggaatccgagaacaagtttagcgaatccgatatgatcgcagttctttgggaaatgatatttaggggaactgacacggtggcaattatgttggaatggattctggctaggatggtgttacacccggacatacaagcaagagcgcaatccgaaatcgatagtgttgtcggctcgggtagacccatatccgatgcggatatcccgaatctcccttacctccatgccattgtaaaagaaaccctacgtgtgcacccaccaagcccacttctgtcatgggcccggctggcaatccatgacacccaagtgggtccgcacatggtancggccgggacaacggccaagggcaatatgtgggccaaaacccatgatgatcaaatnctggggtttgngcccgnaaggntcaacccaaattggtttaagaacc

SEQ ID NO: 39 - protein, 131 aa, Parthenium argentatum Grey
  UNSURE (114): Xaa = any amino acid
VDVLLDLESENKFSESDMIAVLWEMIFRGTDTVAIMLEWILARMVLHPDIQARAQSEIDSVVGSGRPISDADIPNLPYLHAIVKETLRVHPPSPLLSWARLAIHDTQVGPHMVXAGTTAKGNMWAKTHDDQ

SEQ ID NO: 40 - DNA, 1687 bp, Alstroemeria caryophylla
tgccaatgtcgccgccctcaaccctcgccgactcccccctcccctacctcccgacccccatcatcgcctcccctctcctcgccgtcctcgccgccctactcttcgtcttcctcgcccccggcggccccgcctggtccctctcccgctcccgccgccacgccatccccggcccccctggcttcctcctcgctctctccggcccctccgcccaccgctccctcgccgccgtctcctcctccctccgcgccctccccctcctctccttctccctcggcctcacccgcttcattgtctcctcccacccctccaccgccaaggacatcctctccagctccgccttcgccgaccgccccatcaaggactccgcctacggcctcctcttccaccgcgccatgggcttcgcccccttcggtgactactggcgcaacctccgccgcatctccgccacccacctcttcagccccaagcgcctctccgcctccgcccccctccgccgcgacatcggcctccgcgccgtctcccacgtcgcctccctcatggccacccacggcgaggtcgagatcaagcgcctcctccacttcgcctccctcaacaacgtcatggccagcgtgttcggccgcgtctacgacttcgccacccgggacggcctcgagctcgaggccttggtcagcgaggggtacgagctgctgggcgtcttcaactggggcgaccatttcccgcttgttgcctggtttgacttccagggggtcaggcggaggtgcaaggccctcgtcagccgcgtcaacgtctttgtcggccgcataatcgacgagcaccgcagcaggcgggcgagcggctccgtcagcgacggcgccgtagacttcgtcgacgtcctgctcgacgagaagctctccgattccgacatggtggcggttctctgggagatgatctttcgcgggacggatacggtggccatcctgctggagtggatcatggcgaggatggtgctgcacccggaaatccaagccaaagctcaagccgagatcgacgccgttgtgggcggtgagaggtcggtggccgacgccgacgtcgccaacctcccttacctccaagccatcgtcaaggagtcgctgaggatgcacccccccggcccgctgctctcctgggctcgcctcgcagtccatgacgtgcacgtcgggggccacttcgtcccggccggcacgaccgcgatggtgaacatgtgggccatagcgcacgacgggaacatctggccggagccggaggtgttcaacccggagaggtttgtggagcaggatgtgagcattctgggctcggatctccggctggcgccgttcgggtcggggaggagggtgtgtcccggcaaggcgatggggctggccaccgcgcatctctggctggctcagctgcttcagagcttcaagtgggtggcttccgacaatggcgttgatctctcggaaaacttgaagatgtcccttgagatgaaggtccctctcgtgtgcaaggctgttgcgaggcgctgaatggtctggttctctctctttaggttttagtgggtttttagctaactctgtggcttgtttgaactgcatcttggaggtggcggtgctgcactcccctccatggttttgtaacttggtagttaaagcaatggcctcccttttaacgcttaaaaaaaaaaaaaaaaaaa

SEQ ID NO: 41 - protein, 504 aa, Alstroemeria caryophylla
MSPPSTLADSPLPYLPTPIIASPLLAVLAALLFVFLAPGGPAWSLSRSRRHAIPGPPGFLLALSGPSAHRSLAAVSSSLRALPLLSFSLGLTRFIVSSHPSTAKDILSSSAFADRPIKDSAYGLLFHRAMGFAPFGDYWRNLRRISATHLFSPKRLSASAPLRRDIGLRAVSHVASLMATHGEVEIKRLLHFASLNNVMASVFGRVYDFATRDGLELEALVSEGYELLGVFNWGDHFPLVAWFDFQGVRRRCKALVSRVNVFVGRIIDEHRSRRASGSVSDGAVDFVDVLLDEKLSDSDMVAVLWEMIFRGTDTVAILLEWIMARMVLHPEIQAKAQAEIDAVVGGERSVADADVANLPYLQAIVKESLRMHPPGPLLSWARLAVHDVHVGGHFVPAGTTAMVNMWAIAHDGNIWPEPEVFNPERFVEQDVSILGSDLRLAPFGSGRRVCPGKAMGLATAHLWLAQLLQSFKWVASDNGVDLSENLKMSLEMKVPLVCKAVARR

SEQ ID NO: 42 - protein, 537 aa, Arabidopsis thaliana
MTIDMYLSFASRSGSSPFPSLELCLSIFLFISLFVFWLTPGGFAWALYKARFHTRPESKTGPAIPGPSGLPIFGLLLAFVNNALTHRILANIADTCKAKALMAFSVGSTRFVITSEPETAKELLNSSAFADRPVKESAYELLFDRAMGFAPFGDYWRELRRISSTHLFSPKRIFSSGESRRKIGQNMVGEIKNAMECYGEVHIKKILHFGSLNNVMSSVFGKTYNFNEGIVYSKESNELEHLVSEGYELLGIFNWSDHFPGMRWLDLQGVRRRCRSLVGRVNVFVGKIINDHKSKRSLRDNPEESTYDDDFVDVLLGMHGNSKLSDSDMIAVLWEMIFRGTDTVAILLEWILARMVLHPDIQAKAQAEIDCIVGDSGRQVTDSDLPKLPYVRAIVKETLRMHPPGPLLSWARLSIHDTQIGTHFIPAGTTAMVNMWAITHDEKVWPEAHEYKPERFLGAQESNNFPIMGSDLRLAPFGAGRRVCPGKSMGLATVELWLAQLLGSYKWVSCGEVDLSETLKLSLEMKNTLVCKAIPRG

SEQ ID NO: 43 - protein, 426 aa, Phalaenopsis sp. SM9108
MAFSVGLTRFIVSSHPKTAKEILSSPAFADRPIKESAYELLFNRAMGFAPFGDYWRNLRRISSTYLFSPRRVSSFEKQRSEIGEGMVRDMKRMMERNGVVEVRRMLHYGSLNNIMLTVFGKKFDFAKDEGLELELILKEGYELLGIFNWGDHLPLLGWLDLQGVRRRCRTLVAKVNVFVKKIIDEHKRRANGVGIDEGEGEDFVDVLLGLEEKDRLSESDMVAVLWEMIFRGTDTVAILLEWTLARMVLHPDIQSKAQVEIDSVVDSSRPVLDSDIQRLPYLQSIVKETLRMHPPGPLLSWARLAIHDVPVDGHMIPAGTTAMVNMWAITHDECNWAEPNKFNPDRFIDEDVNILGSDLRLAPFGSGKRVCPGKTMALAAVHLWLAQLLKSFKLLPSRNGVDLSECLKMSLEMKNPLVCVAVPRFE

SEQ ID NO: 44 - protein, 534 aa, Arabidopsis thaliana
MATKLDTSSLLLALLSKCSLLTQTNLALSLLVASLASLALSLFFWSHPGGPAWGKYFLHRRRQTTVIPGPRGLPFVGSMSLMSNTLAHRCIAATAEKFRAERLMAFSLGETRVIVTCNPDVAKEILNSPVFADRPVKESAYSLMFNRAIGFAPYGVYWRTLRKIASNHLFSPKQIKRSETQRSVIANQIVKCLTKQSNTKGLCFARDLIKTASLNNMMCSVFGKEYELEEEHEEVSELRELVEEGYDLLGTLNWTDHLPWLSEFDPQRIRSRCSNLVPKVNRFVNRIISDHREQTRDSPSDFVDVLLSLDGPDKLSDPDIIAVLWEMIFRGTDTVAVLIEWILARMVLHPDIQSTVHNELDQIVGRSRAVEESDVVSLVYLTAVVKEVLRLHPPGPLLSWARLAITDTIIDGRRVPAGTTAMVNMWAIAHDPHVWENPLEFKPERFVAKEGEVEFSVLGSDLRLAPFGSGRRVCPGKNLGLTTVTFWTATLLHEFEWLTPSDEKTVDLSEKLRLSCEMANPLAAKLRPRRSFSV

SEQ ID NO: 45 - protein, 523 aa, Glycine max
MTSHIDDNLWIIALTSKCTQENLAWVLLIMGSLWLTMTFYYWSHPGGPAWGKYYTYSPPLSIIPGPKGFPLIGSMGLMTSLAHHRIAAAAATCRAKRLMAFSLGDTRVIVTCHPDVAKEILNSSVFADRPVKESAYSLMFNRAIGFASYGVYWRSLRRIASNHLFCPRQIKASELQRSQIAAQMVHILNNKRHRSLRVRQVLKKASLSNMMCSVFGQEYKLHDPNSGMEDLGILVDQGYDLLGLFNWADHLPFLAHFDAQNIRFRCSNLVPMVNRFVGTIIAEHRASKTETNRDFVDVLLSLPEPDQLSDSDMIAVLWEMIFRGTDTVAVLIEWILARMALHPHVQSKVQEELDAVVGKARAVAEDDVAVMTYLPAVVKEVLRLHPPGPLLSWARLSINDTTIDGYHVPAGTTAMVNTWAICRDPHVWKDPLEFMPERFVTAGGDAEFSILGSDPRLAPFGSGRRACPGKTLGWATVNFWVASLLHEFEWVPSDEKGVDLTEVLKLSSEMANPLTVKVRPRRG

SEQ ID NO: 46 - protein, 530 aa, Arabidopsis thaliana
MATKLESSLIFALLSKCSVLSQTNLAFSLLAVTIIWLAISLFLWTYPGGPAWGKYLFGRLISGSYKTGNVIPGPKGFPLVGSMSLMSSTLAHRRIADAAEKFGAKRLMAFSLGETRVIVTCNPDVAKEILNSPVFADRPVKESAYSLMFNRAIGFAPHGVYWRTLRRIASNHLFSTKQIRRAETQRRVISSQMVEFLEKQSSNEPCFVRELLKTASLNNMMCSVFGQEYELEKNHVELREMVEEGYDLLGTLNWTDHLPWLSEFDPQRLRSRCSTLVPKVNRFVSRIISEHRNQTGDLPRDFVDVLLSLHGSDKLSDPDIIAVLWEMIFRGTDTVAVLIEWILARMVLHPDMQSTVQNELDQVVGKSRALDESDLASLPYLTAVVKEVLRLHPPGPLLSWARLAITDTIVDGRLVPAGTTAMVNMWAVSHDPHVWVDPLEFKPERFVAKEGEVEFSVLGSDLRLAPFGSGRRICPGKNLGFTTVMFWTAMMLHEFEWGPSDGNGVDLSEKLRLSCEMANPLPAKLRRRRS

SEQ ID NO: 47 - protein, 517 aa, Arabidopsis thaliana
MSPEAYVLFFNSFNLVTFEAFASVSLIIATVAFLLSPGGLAWAWTGSSKSRVSIPGPSGSLSVFSGSNPHRVLAALAKRFKASPLMAFSVGFSRFVISSEPETAKEILSSSAFADRPVKESAYELLFHRAMGFAPYGEYWRNLRRISSTHLFSPRRIASFEGVRVGIGMKMVKKIKSLVTSDACGEVEVKKIVHFGSLNNVMTTVFGESYDFDEVNGKGCFLERLVSEGYELLGIFNWSDHFWFLRWFDFQGVRKRCRALVSEVNTFVGGIIEKHKMKKGNNLNGEENDFVDVLLGLQKDEKLSDSDMIAVLWEMIFRGTDTVAILVEWVLARMVLHQDIQDKLYREIASATSNNIRSLSDSDIPKLPYLQAIVKETLRLHPPGPLLSWARLAIHDVHVGPNLVPAGTIAMVNMWSITHNAKIWTDPEAFMPERFISEDVSIMGSDLRLAPFGSGRRVCPGKAMGLATVHLWIGQLIQNFEWVKGSCDVELAEVLKLSMEMKNPLKCKAVPRNVGFA

SEQ ID NO: 48 - DNA, 29 bp, synthetic construct
agaattcttcccatggcgctctcctccat

SEQ ID NO: 49 - DNA, 28 bp, synthetic construct
agaattctaggccctagccacggccttg

SEQ ID NO: 50 - DNA, 26 bp, synthetic construct
aggtctcccatggcgctctcctccat

SEQ ID NO: 51 - DNA, 30 bp, synthetic construct
atcatgatctaggccctagccacggccttg

SEQ ID NO: 52 - DNA, 27 bp, synthetic construst
agcggccgcttcccatggcgctctcct

SEQ ID NO: 53 - DNA, 27 bp, synthetic construct
agcggccgctcaggccctagccacggc

SEQ ID NO: 54 - DNA, 32 bp, synthetic construct
gtttcataatgaaattgactctttttcagtaa

SEQ ID NO: 55 - DNA, 31 bp, synthetic construct
gcaaataattatttctatatacaggacaggc

SEQ ID NO: 56 - DNA, 31 bp, synthetic construct
tagctttagagtacatttcttagatacggca

SEQ ID NO: 57 - DNA, 32 bp, synthetic construct
ttactttgagcgtgccaagcagtataatttct

SEQ ID NO: 58 - DNA, 48 bp, synthetic construct
aaggagaggacgctgtctgtcgaaggtaaggaacggacgagagaaggg

SEQ ID NO: 59 - DNA, 52 bp, synthetic construct
ctctcccttctcgaatcgtaaccgttcgtacgagaatcgctgtcctctcctt

SEQ ID NO: 60 - DNA, 29 bp, synthetic construct
cacccgttctcggagcactgtccgaccgc

SEQ ID NO: 61 - DNA, 30 bp, synthetic construct
atataggcgccagcaaccgcacctgtggcg

SEQ ID NO: 62 - DNA, 30 bp, synthetic construct
cgaatcgtaaccgttcgtacgagaatcgct

SEQ ID NO: 63 - DNA, 20 bp, synthetic construct
ctgaaccatcttggaaggac

SEQ ID NO: 64 - DNA, 20 bp, synthetic construct
acttgcaagtctgggaagtg

SEQ ID NO: 65 - DNA, 21 bp, synthetic construct
attcaggctgcgcaactgttg

SEQ ID NO: 66 - DNA, 20 bp, synthetic construct
ctgcaaggcgattaagttgg

SEQ ID NO: 67 - DNA, 19 bp, synthetic construct
gggttttcccagtcacgac

SEQ ID NO: 68 - DNA, 24 bp, synthetic construct
tgagttagctcactcattagggac

SEQ ID NO: 69 - DNA, 21 bp, synthetic construct
gcttccggctcgtatgttgtg

SEQ ID NO: 70 - DNA, 19 bp, synthetic construct
gaccatgattacgccaagc

SEQ ID NO: 71 - DNA, 16 bp, synthetic construct
  misc_feature (3)..(3): w = a or t
tgwgnagwancasaga

SEQ ID NO: 72 - DNA, 16 bp, synthetic construct
  misc_feature (3)..(3): w = a or t
agwgnagwancawagg

SEQ ID NO: 73 - DNA, 16 bp, synthetic construct
  misc_feature (3)..(3): w = a or t
cawcgncnganasgaa

SEQ ID NO: 74 - DNA, 16 bp, synthetic construct
  misc_feature (3)..(3): s = c or g
tcstncgnacntwgga

SEQ ID NO: 75 - DNA, 16 bp, synthetic construct
  Unsure (1)..(1): n = a, c, g, or t
ngtcgaswganawgaa

SEQ ID NO: 76 - DNA, 16 bp, synthetic construct
  Unsure (3)..(3): n = a, c, g, or t
gtncgaswcanawgtt

SEQ ID NO: 77 - DNA, 16 bp, synthetic construct
  misc_feature (1)..(1): w = a or t
wgtgnagwancanaga

SEQ ID NO: 78 - DNA, 21 bp, synthetic construct
gggaagcgttcgcgaagtgag

SEQ ID NO: 79 - DNA, 23 bp, synthetic construct
agcggataacaatttcacacagg

SEQ ID NO: 80 - protein, 6 aa, conserved sequence motif
  UNSURE (2)..(2): Xaa = any amino acid
SXGLTR

SEQ ID NO: 81 - protein, 11 aa, conserved sequence motif
  UNSURE (5)..(5): Xaa = any amino acid
LLFHXAMGFAP

SEQ ID NO: 82 - protein, 7 aa, conserved sequence motif
  UNSURE (3)..(3): Xaa = any amino acid
MXTVFGK

SEQ ID NO: 83 - protein, 48 aa, conserved sequence motif
  UNSURE (4)..(4): Xaa = any amino acid
EGYXLLGXFNWXDHXPXLXXLDXQGXRXRCXXLVXKVXXFVGXXIXEH

SEQ ID NO: 84 - protein, 7 aa, conserved sequence motif
DFVDVLL

SEQ ID NO: 85 - protein, 15 aa, conserved sequence motif
AVLWEMIFRGTDTVA

SEQ ID NO: 86 - protein, 4 aa, conserved sequence motif
MARM

SEQ ID NO: 87 - protein, 6 aa, conserved sequence motif
IQAKAQ

SEQ ID NO: 88 - protein, 19 aa, conserved sequence motif
  UNSURE (7)..(7): Xaa = any amino acid
VKETLRXHPPGPLLSWARL

SEQ ID NO: 89 - protein, 9 aa, conserved sequence motif
GTTAMVNMW

SEQ ID NO: 90 - protein, 16 aa, conserved sequence motif
  UNSURE (9)..(9): Xaa = any amino acid
DLRLAPFGXGRRXCPG

SEQ ID NO: 91 - protein, 7 aa, conserved sequence motif
  UNSURE (3)..(3): Xaa = any amino acid
PLXCKAV

SEQ ID NO: 92 - DNA, 1585 bp, Hordeum vulgare
gcggccgcgagctcaattaaccctcactaaagggagtcgactcgatctttccatggttaccggcccggaggactccctcctcttgctcttcctcccggctaccaccctgctcccaccccttctcgccgtgctcctcctcgccgcctccctcctgtggctgtcaccgggcggtccggcgtgggctttgtcactctgccgtcgcccgccgccaggcccaccgggcgtggtcaccgcgctctccagccccgtggcgcaccgcgtcatggctacgctgtcacgctccgtccgcggcggcgcggcattgatgtccttctccgtcggcctcacccgcgtcgtcgtgtcgagcaggcaagatacggcgcgtgagatactcgtcaacccggcgttcggcgaccggccggtgaaggacgcggcgcgccacctcctcttccaccgcgccatgggttttgccccgtcgggcgacgcgcactggcgtgcgctgcgccgtctcgccgcggcgcacctcttcggccctcgccgtgtggcggcctccgcaccccaccgttcctctattggggcgcgcatgctcggcgacgtcgcctccatcatggcccgccacggcgaggtcgctcctcggaggttcctgcacgcggcgtccctcaaccacgtcatggccgtcgtcttcggcaagcgctacgacgacttcacaagccaagaaggagtcgttgtggaggagatggtaaacgaagggtacgacctcctcggcacgttcaactgggcagatcacctgccattcctcaagtgcctcgatctccagggcgtgcggcgccggtgcaacaggttagtccggcaagtggaggcgtacgtcggtaacatcatacaggagcacaaggcgaggcgcgacagtgcatcaggcattgcggatgagctctccggcgacttcgtcgatgtgctcctcggcctcgacggagaagacaagatgtcagagtccgacatgatcgccgttctttgggagatgatctttagagggacggacacggtggcgatcttgatggagtggattatggcgaggatggtgctgcacccggagatccagtcgaaggcccgggcggagcttgacgccgtggtgggccggggcagggccgtgacggacgaggacgtgtcgaggctcccctacatccagtgcatcgtcaaggagacgctgcgcatgcacccgccgggcccgctcctctcatgggcgcggctggccgtgcacgacgcgcacgtcggcggccacctcgtgccggccggcacgacggcgatggtgaacatgtgggccatcgcgcacgacgcggcggtgtggcccgagccggagctgttccggccggagcggttcatggaggaggacgtgagcgtgctgggcagcgacctccgcctggccccgttcggcgccgggcggcgcgtgtgccccgggaagatgctggccctcgccaccgtccacctctggctcgcgcagctgcttcaccggttcgagtgggctccctcggggagcgtcgacctgtcagagcgcctcaagatgtcactggagatggccacgccgctggtctgcaaggccgtcgctcgctag

SEQ ID NO: 93 - protein, 510 aa, Hordeum vulgare
MVTGPEDSLLLLFLPATTLLPPLLAVLLLAASLLWLSPGGPAWALSLCRRPPPGPPGVVTALSSPVAHRVMATLSRSVRGGAALMSFSVGLTRVVVSSRQDTAREILVNPAFGDRPVKDAARHLLFHRAMGFAPSGDAHWRALRRLAAAHLFGPRRVAASAPHRSSIGARMLGDVASIMARHGEVAPRRFLHAASLNHVMAVVFGKRYDDFTSQEGVVVEEMVNEGYDLLGTFNWADHLPFLKCLDLQGVRRRCNRLVRQVEAYVGNIIQEHKARRDSASGIADELSGDFVDVLLGLDGEDKMSESDMIAVLWEMIFRGTDTVAILMEWIMARMVLHPEIQSKARAELDAVVGRGRAVTDEDVSRLPYIQCIVKETLRMHPPGPLLSWARLAVHDAHVGGHLVPAGTTAMVNMWAIAHDAAVWPEPELFRPERFMEEDVSVLGSDLRLAPFGAGRRVCPGKMLALATVHLWLAQLLHRFEWAPSGSVDLSERLKMSLEMATPLVCKAVAR

SEQ ID NO: 94 - DNA, 1758 bp, Zea mays
atgcagttattaggactgccaaatacctacctgcgatttaaactgcaaacagtaaattatttggcgtgcagttgccagatcagcagccattttcaccgcactccccccgccccttttaaaagctccctccctctcaacactctacacacaccagctccactgcatcaaaacccctcatcaccctgcagcctgcactcatcagacatggtgctcaccatggccagcggccaagaggactcgctcctcctcccgaccacctccccactgccgcccctcatggcagtgttcatcctagccgccgtcctcctgtggctctcccccggcggtcctgcgtgggcgctctcccgctgccgccgcccgccgcccgggccaacgggcgtggtcaccgcgctctccagccccgtggcgcaccgcaccctggcggcgctgtcccacgccgtagacggcggcaaggcactgatggccttctcggtcgggctgacccgtctcgtcgtgtcgagccagcccgatacggcgcgcgagatcctcgccagccccgcgttcggcgaccgccccgtcaaggacgcggcgcgccacctgctcttccaccgcgccatgggcttcgcgccctccggagacgcgcactggcgcgggctccgccgcctcgccgccaaccacctgttcggcccgcgccgcgtggcgggtgccgcgcaccaccgcgcctccatcggcgaggccatggtcgccgacgtcgccgctgccatggcgcgccacggcgaggtccctctcaagcgcgtgctgcatgtcgcatctctcaaccacgtcatggccaccgtgtttggcaagcgctacgacatgggcagccgagagggcgcccttctggacgagatggtggccgagggctacgacctcctgggcacgttcaactgggctgaccacctgccattgctcaagcatctcgacccccagggcgtgcgccgccggtgcaacaggctggtccgaaaggtcgaatcgttcgttggcaagatcatcttggagcacagggcgcggcgcgcaaatggaggagtcgtgggcgatgagtgcatgggtgacttcgtcgacgtccttcttggcctcgagggagaggagaagctgtcagatgcggacatgatcgctgttctttgggagatggtcttcagaggcgccgacaccgtggcgatcttgatggagtgggtcatggcgaggatggcgctgcacccggacatccaggcgaaggcccaggcggagctggacggcgtcgtgggcatcgggcgcggcgtggcggacgccgacgtcgccagcctaccctacatccagtgcatcgtgaaggagacgctgcgcatgcacccgccaggcccgctcctgtcgtgggcgcgcctcgccgtccacgacgcgcacgtcggcggccacctggtccccgccggcaccacagccatggtgaacatgtggtccatcgcgcacgaccccgccatctgggccgagccggagaagttccgccccgagcggttccaggaggaggacgtgagcgtcctcgggagcgacctccgcctggcccccttcggcgccgggcgccgcgcctgccccggcaagatactggccctcgccaccacccacctctgggtcgcccagcttctgcacaagttcgagtgggccgccggcgggggcgtcgacctgtcggagcgcctgagcatgtcgctggagatggccacgccgctggtgtgcaaggccgtacccagggttcagggccaagcggcctcctag

SEQ ID NO: 95 - protein, 585 aa, Zea mays
MQLLGLPNTYLRFKLQTVNYLACSCQISSHFHRTPPAPFKSSLPLNTLHTPAPLHQNPSSPCSLHSSDMVLTMASGQEDSLLLPTTSPLPPLMAVFILAAVLLWLSPGGPAWALSRCRRPPPGPTGVVTALSSPVAHRTLAALSHAVDGGKALMAFSVGLTRLVVSSQPDTAREILASPAFGDRPVKDAARHLLFHRAMGFAPSGDAHWRGLRRLAANHLFGPRRVAGAAHHRASIGEAMVADVAAAMARHGEVPLKRVLHVASLNHVMATVFGKRYDMGSREGALLDEMVAEGYDLLGTFNWADHLPLLKHLDPQGVRRRCNRLVRKVESFVGKIILEHRARRANGGVVGDECMGDFVDVLLGLEGEEKLSDADMIAVLWEMVFRGADTVAILMEWVMARMALHPDIQAKAQAELDGVVGIGRGVADADVASLPYIQCIVKETLRMHPPGPLLSWARLAVHDAHVGGHLVPAGTTAMVNMWSIAHDPAIWAEPEKFRPERFQEEDVSVLGSDLRLAPFGAGRRACPGKILALATTHLWVAQLLHKFEWAAGGGVDLSERLSMSLEMATPLVCKAVPRVQGQAAS

SEQ ID NO: 96 - DNA, 1545 bp, Zea mays
atggacgccaccctcagcaccacgaccacccaggactccctactcttcctcctcccttcagccgccaccttgctctccccgctcctgaccgtgctcctcgtagccgtctcgctgctctggctcttcccgggcgggcccgcgtgggcgttcgtctccaggtcccgcgcgacgccgccgggcgcgccgggcctggtcaccgcgctcgcgggccccgcggcgcaccgcgccctcgcgtcgctgtcccggtcccttcccggcggcgccgcgctgtcggccttctccgtcggcctcacgcgcctcgtcgtagcgagccagccggacacggcgcgggagctcctggccagcgccgccttcgccgaccgccccgtgaaggacgcggcgcgggggctcctcttccaccgcgccatgggctttgccccgtcgggcgactactggcgcgcgcttcggcgcatcagctccgcgtacctcttcagcccgcgcagcgtggccgcggcgggcccgcgccgcgccgccatcggcgagcgcatgctgcgggacctctccggcgcggccggacgagaggtcgtcatgcggcgcgtgctccacgcggcatccctggaccacgtcatggccaccgtgttcggcgcgcgctacgacgccgccagcccggagggcgcggagctggaggagatggtgaaggaagggtacgacctgctcggcatgttcaactggggcgaccacctgccgctgctcaggtggctggacctgcagggcgtcaggaggcggtgcaggagcctggtgggcagagtcaacgtgttcgtggccaggatcatcgaagagcacaggcagaagaaggacgacgccattggagagccggcggccgccggagacttcgtcgacgtcttgctgggactggagggcgaggagaagctgtcggactccgacatgatcgctgtcctctgggagatgatctttcgagggaccgacacggtggcgatcctgctggagtgggtgatggcgcggatggtgctgcacccgggcatccagtccaaggcgcaggcggagctggacgccgtggtgggccgcggccgcgccgtttgcgacgccgacgtggcccgcctgccctacctgcagcgcgtcgtgaaggagacgctccgcgtgcacccgccgggcccgctgctctcgtgggcgcgcctggccgtgcgcgacgcggtggtcggcggccacgtggtccccgcgggcaccacggccatggtcaacatgtgggccatcgcgcacgaccccgcggtgtggccggagccctccgctttccggcccgagcggttcgaggtggaggacgtgagcgtgctgggcggcgacctccgcctcgcgcccttcggcgccggccggcgcgtgtgcccgggcaagacgctggcgctcgccactgtccacctctggctcgcgcagctgctgcaccgcttccggtgggcgccggccgacggccgcggcgtcgacctggcggagcgcctcggcatgtccctggagatggagaagcccctcgtgtgcaagcccacgccgaggtggtga

SEQ ID NO: 97 - protein, 514 aa, Zea mays
MDATLSTTTTQDSLLFLLPSAATLLSPLLTVLLVAVSLLWLFPGGPAWAFVSRSRATPPGAPGLVTALAGPAAHRALASLSRSLPGGAALSAFSVGLTRLVVASQPDTARELLASAAFADRPVKDAARGLLFHRAMGFAPSGDYWRALRRISSAYLFSPRSVAAAGPRRAAIGERMLRDLSGAAGREVVMRRVLHAASLDHVMATVFGARYDAASPEGAELEEMVKEGYDLLGMFNWGDHLPLLRWLDLQGVRRRCRSLVGRVNVFVARIIEEHRQKKDDAIGEPAAAGDFVDVLLGLEGEEKLSDSDMIAVLWEMIFRGTDTVAILLEWVMARMVLHPGIQSKAQAELDAVVGRGRAVCDADVARLPYLQRVVKETLRVHPPGPLLSWARLAVRDAVVGGHVVPAGTTAMVNMWAIAHDPAVWPEPSAFRPERFEVEDVSVLGGDLRLAPFGAGRRVCPGKTLALATVHLWLAQLLHRFRWAPADGRGVDLAERLGMSLEMEKPLVCKPTPRW

SEQ ID NO: 98 - DNA, 1557 bp, Zea mays
atggacgccacccaggactccctcctcttcctcttcccggccgccgccaccttactctccccgctccttgccgtgctcctcgcagctctctcgctgctctggctctacccgggcggtcccgcgtgggcgctcatctctaggtcccgcgcgacgccgcccggcacgccggacgtggtcaccgcgctcgcgggtcccgccgcgcaccgcgccctggcgtcgctgtcgcagtcgctgcccggccgcgccgcgctgtcggccttctccgtaggtctcacgcgccttgtcgtggccagccagccggacacggtgcgggagctcctggccagcgccgccttcgccgaccgccccatcaaggacgcggcgcgggggctcctcttccaccgcgccatgggcttcgccccctccggcgactactggcgcgcgctgcggcgcatcagctccgcgtacctcttcagcccgcgcagcgtgtccgcaacggccccgcgtcgtgtcgccatcggcgagcgcatgctgcgggacctctcggccgcgcccggcggcgaggtcgtcatgcggcgcgtgctccacgcggcctccctcgaccacgtcatggccaccgtgttcggcgcgcactacgacgccgccagcccggagagcgcggagctggaggagatggtgaaggaagggtacgacctgctcggcttgttcaactggggcgaccacctgccgttgctcaggtggctggacctgcaaggcgtcaggaggaggtgcaggagcctggtgagcagagtgaacgtgttcgtggcgaggatcatcgaagagcacaggcggaagaagaaggaggccgccagtggcgagtcggtcgccggagacttcgtcgacgtcttgctgggattgcagggcgaggagaagctgtcggactttgagagttgtgttaacacggactccgacatgatcgctgtcctctgggagatgatctttcgaggcaccgacacggtcgcgatcctgctggagtgggtgatggcgcggatggtgctgcacccgggcatccagtccaaggcgcaggcggagctggacgccgtcgtgggtcgcggccgcgtgtccgacgccgatgtggtccgcctgccctacctccagcgcgtcgtaaaggagacgctccgcgtgcacccgcccggcccgctgctgtcgtgggcgcgcctggccgtgcacgacgcggtggtcggcggccacctggtccccgccggcaccacggccatggtgaacatgtgggcgatcgcgcacgaccccgcggtgtggccggagccctccgcgttccgccccgagcggttcgaggaggagtacgtgagcgtgctgggcggcgacctccggttcggcgccggccggcgcgtgtgccccggcaagacgctggcactcgccactgtccacctctggctcgcgcagctgctgcaccgcttccagtgggcggcgtcgacctggcggagcgactcggcattgggcggcgtcgacctggcggagcgactcggcatgtcgctggagatggagaagcccctcgtgtgcaagcccacgccgaggtggtaa

SEQ ID NO: 99 - protein, 518 aa, Zea mays
MDATQDSLLFLFPAAATLLSPLLAVLLAALSLLWLYPGGPAWALISRSRATPPGTPDVVTALAGPAAHRALASLSQSLPGRAALSAFSVGLTRLVVASQPDTVRELLASAAFADRPIKDAARGLLFHRAMGFAPSGDYWRALRRISSAYLFSPRSVSATAPRRVAIGERMLRDLSAAPGGEVVMRRVLHAASLDHVMATVFGAHYDAASPESAELEEMVKEGYDLLGLFNWGDHLPLLRWLDLQGVRRRCRSLVSRVNVFVARIIEEHRRKKKEAASGESVAGDFVDVLLGLQGEEKLSDFESCVNTDSDMIAVLWEMIFRGTDTVAILLEWVMARMVLHPGIQSKAQAELDAVVGRGRVSDADVVRLPYLQRVVKETLRVHPPGPLLSWARLAVHDAVVGGHLVPAGTTAMVNMWAIAHDPAVWPEPSAFRPERFEEEYVSVLGGDLRFGAGRRVCPGKTLALATVHLWLAQLLHRFQWAASTWRSDSALGGVDLAERLGMSLEMEKPLVCKPTPRW

SEQ ID NO: 100 - DNA, 1155 bp, Oryza sativa
atggggtcgctgatgtcctgcatctccgggcaggcaccgtcggcgtcgccgccgccggtggcgaagcggcggtcatccgtgtcgtcgcgccgcggcggcggcggcggaggcgccaaggcggtggccatcgacgaggaggcgctggcggcggcggcggcgctggtgctggggcagaggagctcgttcggcggaggcgggggtggaggcggaggcgcgttcgagcggtcggcgtcggtgcggtacgcggcgaggcggcagcagcagcagcagggcccgccgctgccgaggagctccagcacgcgcccccgctccctcgccgacccggagctccacccgcagcagcttctcgccaaggatttgaacactaaagatcttgaaaccaacatcattgttcttgttcatggaggaggttttggtgcttggtgttggtacaagactatagcacttcttgaggatagtgggttcagagtcaatgctattgacttaacaggttccgggattcattcgtatgatacaaacaagattagcagtctcacgcagtatgctgagccgcttacatcttaccttaaaagcctaggtgacaacgaaaaggtgattttggttggacatgattttggtggtgcttgtatatcctacgcaatggagatgtttccatcaaaagttgcgaaggctgttttcctttgtgcagcaatgctgaaaaatgggcatagtactcttgatatgtttcaacaacagatggatacaaatggtacactccaaagggcgcaggaatttgtatattccaatggcaaggagcagcctcccaccgctatcaatatagagaagtctttactgaaacatttgttgttcaaccaaagcccctctaaggatgtatctttggcttcagtgtccatgagacctatcccctttgctcctgtgctggagaagctggtcctaacagaagagaagtacggatcggtgcggcgattctacgtcgaaaccacagaagacaatgccattccacttcatcttcagcaaggtatgtgcgacatgaacccgcccgagaaggttcttcggttgaaaggctcggatcatgccccattcttctccaagccacaagctctgcacaagacccttgtagagatagcaaccatgccaccagtcaaggcatcatga

SEQ ID NO: 101 - protein, 384 aa, Oryza sativa
MGSLMSCISGQAPSASPPPVAKRRSSVSSRRGGGGGGAKAVAIDEEALAAAAALVLGQRSSFGGGGGGGGGAFERSASVRYAARRQQQQQGPPLPRSSSTRPRSLADPELHPQQLLAKDLNTKDLETNIIVLVHGGGFGAWCWYKTIALLEDSGFRVNAIDLTGSGIHSYDTNKISSLTQYAEPLTSYLKSLGDNEKVILVGHDFGGACISYAMEMFPSKVAKAVFLCAAMLKNGHSTLDMFQQQMDTNGTLQRAQEFVYSNGKEQPPTAINIEKSLLKHLLFNQSPSKDVSLASVSMRPIPFAPVLEKLVLTEEKYGSVRRFYVETTEDNAIPLHLQQGMCDMNPPEKVLRLKGSDHAPFFSKPQALHKTLVEIATMPPVKAS

SEQ ID NO: 102 - DNA, 1149 bp, Zea mays
atgggttcgctggtgtcctgcctctccgacccctgccagtcggggaacgggtccccgccgccgcaggcgaggcggcgctcctccacctcctcccgcggcggccgtggcggcggcgggagggactccgccaaggcgtcggtgaccatagacgaggaggcgctggccgcggcggcggcgctcgtgctggggcagcggggcgccgccgccgttggcgcgttcgagcggtccgcgtcggtgcggtacgcggccaagcggcacggccagggcccgccgctgccccgcagctgcagcacgcgccccaggtcgctcgctgaccccgagctccagccgcagcagctcctcgccaaggatttgaacaccaaggatttggaaaccagcgtcattgttctcgttcatggaggcggattcggcgcgtggtgttggtacaagactatatcgcttcttgaagacagtgggttcagagttaacgccatcgacttgacaggctccgggatccattcttatgacacgaacaagattagcagtctttcagagtacgctgaaccgcttacgtcttaccttgaaggcttaggtgatgctgaaaaggtaatcttggtggctcatgatcttggtggtgcctgtgtatcctacgcaatggagatgttcccatccaaagttgccaaggccgttttcctctgtgcagcgatgctgacgaacggaaacagtgcccttgacatgttccagcagcagatggacacaaacggtacgctccaaaaggcgcaggcattcgtctactccaacggcaaggaccggcccccgaccgccatcaacgtcgacagggcattgcttagagacttgttgttcaaccagagcccttccaaggacgtgtcgctggcctcggtgtccatgaggcccatccccttcgcccctgtgctggagaagctcgtgctcaccgccgagaactacggctcggtgcggcggttctacgtggagaccacggaggacaacgcgatccctctgcccctccagcagagcatgtgtggcgccaacccaccggagaaggtgctgcggctgaaaggggccgaccacgcacccttcttctccaagccgcaggcgctgcacaagaccctcgtcgagatcgccgccatgccgccggtcggggcttcgtga

SEQ ID NO: 103 - protein, 382 aa, Zea mays
MGSLVSCLSDPCQSGNGSPPPQARRRSSTSSRGGRGGGGRDSAKASVTIDEEALAAAAALVLGQRGAAAVGAFERSASVRYAAKRHGQGPPLPRSCSTRPRSLADPELQPQQLLAKDLNTKDLETSVIVLVHGGGFGAWCWYKTISLLEDSGFRVNAIDLTGSGIHSYDTNKISSLSEYAEPLTSYLEGLGDAEKVILVAHDLGGACVSYAMEMFPSKVAKAVFLCAAMLTNGNSALDMFQQQMDTNGTLQKAQAFVYSNGKDRPPTAINVDRALLRDLLFNQSPSKDVSLASVSMRPIPFAPVLEKLVLTAENYGSVRRFYVETTEDNAIPLPLQQSMCGANPPEKVLRLKGADHAPFFSKPQALHKTLVEIAAMPPVGAS

SEQ ID NO: 104 - DNA, 2022 bp, Zea mays
atcaacaagaattaaattttttattcttaatataatctatgatggcttcagtgatctattctgtacaagtgttacacaattccttttgagtagatggtctgttgcctacgaacgttagttggtccagaatactcggccgctactgaagataggattgctgggggctggggctgaggctgggtgatgccgtggctgtggataaactgacgagaggattggaggacttggaacgggtgaaagagtcatacgtacacggtacacgaccccaataacccccagccggccctatatgtacacgtacacgatacaccgtgtcatgcgctggaaaaaccgaaactcttgcgacgctggaaagtggaacccaccaaaacgaaggctggcagtatgtgtacgctacagggctcctacagcaatggccaatgagaccacgagctcgctggcatgcatcgcagcagcaccggtgccgttttggtgggtcggaggagttaccgctttcggatcgtttttatgcccgggttcgcgggtgtatcgaaccgctaaagcatgacacgacgccacgacgatggtttcttgggtattgctcgcacaccacgcacggctttgatgatactgtgtctttttattgacttcacggtaaattttaccatttgagccgatcttttatttttcttattacgattaatatctatcatggattgttaataagaactctcgttcttttttcgaaagatatttcctgtcttgtttttttagtttactagtcagatatagtttctaaatatcatatggctaattttttaaataaaacacaaaaatatatgtaatctattagttagatgagtataaatatatagccaacaactaagtttcaaaccaccgctaaattgttacatccatcgccgtggtcgtgggccgcctcacccatcaaccgtcggaccagcctagagccaatgcgtggtcgagcggccacgtgagagcgcgactatcgcaaaagctctttgtgcatgtcactcatttatatatattggaagattttttttcccgagatccaacttctattcgaagtatgtcttgcttgcatgcaccaccgcatatccgctagcattatttcacatagtgttgcgcttgcctttcgctttagttctaactagcatttgtatgttgtaacgtaactcattacgcgctaaagtttagtccatattatattgaatgtttggttgtcaactatgagtattaaatatagactaattaaaaactaattacatagattagactaaacggcgagataagtctcttggtttgatattattggtctgtctatatatttacttaaacactttttctaatggtcaaatgctgatttttatcttctctttaagaaataaaatatccgccgtcttatttgattttttttttctgcaaatcaaggtgactctcaactttagaacatctccaagtgactttttatttattagctctctatttaactttctatttatcatcccataacgattattactctatatgtagcatctcactcaaacagactatctatctagtttgactagttaaagtggttagccaagtttgactagttacatagacaatttggagtcgaatatcttggcaagttagataactaatctgttggagagttattttgctgttgagtagccaaaatttggcttcatgagccatttggctagtctattgaaaatgctcttacatgttcatagactaatggtaaaaaatcgttgtttgaaaatattactcttttcgttcttttttatttgtcaccgattaattcaaaaataaattaacgagccacaaatattcgagaacagagttaggcaattgaaatatagcaagtctacataggatcttatcggttattgcccacacataaatcataatgcgtttcacctggataaaaaatcaaggcatttatatcaaaggtaacatgctaatgcgtcattactgttgaaaaagcaggctctcgatcacgatttgattgataatata

SEQ ID NO: 105 - DNA, 2000 bp, Zea mays
cgacaaaactatcaacaggtatatttactaaatgttcttcaaactggctttagaggctagaggtgtagccaaggggatgtttgtttgtgattataatttgtctatattatataatctaacaaatttattttaaattagttgttagtttaatatttattggattatataatctgaatagattataatttcagacaaacaccctaaaatgttttccaaaatagctttagagaccattttgttaaaacagctagtagatggtacgctccatattccacaaggccggtgatagcggctagaaaaataattgttgctccttcccaaaacatgagttatattagtttttgtaaagttaatatacctcaaattataagttattttaacctttttaaaatcaaagcatcttaagtttaatcaaattcgaataataaaacaatactatatataatattaaataaatatcattattttgtcattaattatatttagtatacctattcaatgttataaatcttataatttcattctatgattttaaccgacaaatttgagaagctttgattccttagaaaaaacaaaatggtttataattttaaacggagtgagcctgtggcttgattgcaaatgtggtcgtggaaagccgtcggccgatcggtccccgtccgtattctcttgcatcgttgcgtgcgatggaaaggctactagtgagagctgttggagcggcgggcggcggaagtctagctacggggtccccgccgtcggcgcaagtaccgcgcgtgtaggtggcggcggcgcagacgcactttatacacgggcgggacggggaccggggacgaggactagccagggaggccgcgccgcgccgccgcggcccgcagtcgcctggcgctcgtctgtccgtgtccggtacccccacctgcagcctgcagtatatattagcagcaagtttaaatttcagcggcctcacggttaacgctaataataaccgccacgccgtcgaacgaaatgtgatcgcaggcgagtaatttgtcactgatagtggcctgctgcggccatgcagcgattcctcgaagcacttgctgaatccaaccattctctctcgaatcttcctacttgtacttttcatatgtaaatacctctttattcttcgtatccgtttgaccgtttctaactattctccgtattcagctttcctatacacttcaacttagctatttaactttttacataagtttttagagtttttaaaaaaaatactacattatttatgtaatgcaatacacattgtttttagttaattaaactagaaaaagattgatttcctagttaaaatcactgattaatgaaaagggtgagattagagctttccctaacagagaaaaatattcaaggctcagtgaccagacatacattaaattcacgcgggaaaaggtcgagtgaaccgttggacactgtcttagggcatgtacaatctttaaccatcgaatcggttttctaagtatggcatcaatttattattcttgtttaagtatatatatagaaataacggtagattgtctttatgtcattacagaccagattttgttgaatttgtgatttcatctaacatattcttttattcttagaaccaaaaagtatataatatttttataaattacaacgaactaaagttttagttttagtgtaaaacatatgcgataaccgtagcctaaaaagctaaaattagtaccagcagaatttaaaagagtcccattctttttacgagaacttctcgttaaaagctgaacgccagttgcaaaagcggctacattctctcctttaatcagggaatcagtacaatgcgtttccatttctcctccagccgttactagtgtcatgctctcagcacactggtctgctcgtctgcctcctttgccttcctctatttaaaccctctccgcccccccggacccaaaacccacaccatccagccttcccacctccctcccccccacgccgtcgtc

SEQ ID NO: 106 - DNA, 21 bp, synthetic construct
tcgtgtgcaaggccgtggcta

SEQ ID NO: 107 - DNA, 23 bp, synthetic construct
gcacgatccatttagcacaccag

SEQ ID NO: 108 - DNA, 38 bp, synthetic construct
aattaaccctcactaaagggcacctgctcttccaccac

SEQ ID NO: 109 - DNA, 40 bp, synthetic construct
gtaatacgactcactatagggcgactgcccatttcgtagc

SEQ ID NO: 110 - DNA, 346 bp, synthetic construct
  misc_feature (39)..(39): s = c or g
cacctgctcttccaccacgccatgggcttcgcgccctcsggagacgcgcactggcgcgggctccgccgcctckccgccaaccacctgttcggcccgcgccgcgtggcgggtgccgcgcaccaccgcgcctccatcggcgaggccatggtcgccgacgtcgccgctgccatggcgcgccacggcgaggtccctctcaagcgcgtgctgcatgtcgcgtctctcaaccacgtcatggccaccgtgtttggcaagcgctacgacatgggcagccgagagggcgcccttctggacgagatggtggccgagggctacgacctcctgggcacgttcaactgggctgatcaac

SEQ ID NO: 111 - DNA, 17 bp, synthetic construct
gatcgatggaactgagt

(19) United States
(12) Patent Application Publication
(10) Pub. No.: US 2003/0126645 A1
(43) Pub. Date: Jul. 3, 2003

(54) Alteration of embryo/endosperm size during seed development

(76) Inventors: Rebecca E. Cahoon, Webster Groves, MO (US); Elmer P. Heppard, Wilmington, DE (US); Nobuhiro Nagasawa, Newark, DE (US); Hajime Sakai, Newark, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/163,198

(22) Filed: Jun. 5, 2002

Related U.S. Application Data
(60) Provisional application No. 60/295,921, filed on Jun. 5, 2001.
(60) Provisional application No. 60/334,317, filed on Nov. 28, 2001.

(51) Int. Cl. A01H 5/00; C07H 21/04; C12N 9/02; C12N 9/24; C12N 9/50; C12N 5/04

(52) U.S. Cl. 800/287; 435/200; 435/219; 435/419; 435/69.1; 435/320.1; 536/23.2; 435/189

(57) ABSTRACT

Isolated nucleic acid fragments and recombinant constructs comprising such fragments for altering embryo/endosperm size during seed development are disclosed along with a method of controlling embryo/endosperm size during seed development in plants.

This application claims the benefit of U.S. Provisional Application No. 60/295,921, filed Jun. 5, 2001, the entire contents of which are hereby incorporated by reference, and U.S. Provisional Application No. 60/334,317, filed Nov. 28, 2001, the entire contents of which are hereby incorporated by reference.

### FIELD OF THE INVENTION

The present invention is in the field of plant breeding and genetics and, in particular, relates to recombinant constructs useful for altering embryo/endosperm size during seed development.

### BACKGROUND OF THE INVENTION

Elucidation of how the size of a developing embryo is genetically regulated is important because the final volume of endosperm as a storage organ of starch and proteins is affected by embryo size in cereal crops. Researchers have found that embryo size-related genes contribute to the regulation of endosperm development. Investigation of these genes is important for agriculture because cereal endosperms are the staple diet in many countries. Also, it is important for agriculture because embryos of various crop grains are the source of many valuable nutrients including oil.

The giant embryo (ge) mutation was first described by Satoh and Omura (1981) Jap. J. Breed. 31:316-326. The giant embryo mutant is a potentially useful character for quality improvement in cereals because increased embryo size will result in increased embryo oil and nutrient traits that are desirable for human consumption. Also, the enlargement of embryos would result in increased embryo-related enzymatic activities, which are often important features in the processing of grains. The mutation was genetically mapped to chromosome 7 (Iwata and Omura (1984) Japan. J. Genet. 59: 199-204; Satoh and Iwata (1990) Japan. J. Breed. 40 (Suppl. 2): 268-269), with additional ge alleles also localized to chromosome 7 (Koh et al. (1996) Theor. Appl. Genet. 93:257-261). The ge mutations were analyzed at the morphologic and genetic level by Hong et al. (1994) Development 122:2051-2058. This publication linked the GE gene as being required for proper endosperm development. Since both endosperm and embryo size are affected by the mutation, GE appears to control coordinated proliferation of the endosperm and embryo during development. Beside the morphological change of embryo and endosperm in ge, it was also shown that the ge seed accumulates more oil compared to the wild type (Matsuo et al. (1987) Japan. J. Breed. 37: 185-191; Okuno (1997) In “Science of the Rice Plant” Vol. III, Matsuo et al. eds., Food and agriculture policy research center, Tokyo, Japan, pp433-435).

It has been found that loss-of-function of the GE gene leads to an enlargement of embryonic tissue at the expense of endosperm tissue. This developmental change may be useful in increasing the amount of embryo-specific metabolites such as oil in seed-bearing plants. Despite the extensive genetic and morphological characterization of the GE gene there has been no molecular analysis of the nucleic acid encoding this protein. Indeed, the identity of the protein encoded by GE has not been reported. A better understanding of the GE gene, and the protein it encodes, will be required for a complete understanding of the process controlling embryo size in rice.

### SUMMARY OF THE INVENTION

This invention concerns an isolated nucleotide fragment comprising a nucleic acid sequence selected from the group consisting of:

(a) a nucleic acid sequence encoding a cytochrome P450 polypeptide associated with controlling embryo/endosperm size during seed development having an amino acid identity of at least 61% based on the Clustal method of alignment when compared to a second polypeptide selected from the group consisting of SEQ ID NO:2, 7, 11, 19, 27, or 33; or

(b) a nucleic acid sequence encoding a cytochrome P450 polypeptide associated with controlling embryo/endosperm size during seed development having an amino acid identity of at least 65% based on the Clustal method of alignment when compared to a third polypeptide selected from the group consisting of SEQ ID NO:15, 17, 31, 93, 95, 97, or 99; or

(c) a nucleic acid sequence encoding a cytochrome P450 polypeptide associated with controlling embryo/endosperm size during seed development having an amino acid identity of at least 70% based on the Clustal method of alignment when compared to a fourth polypeptide selected from the group consisting of SEQ ID NO:9, 13, 23, 29, 35, or 41; or

(d) a nucleic acid sequence encoding a cytochrome P450 polypeptide associated with controlling embryolendosperm size during seed development having an amino acid identity of at least 77% based on the Clustal method of alignment when compared to a second polypeptide selected from the group consisting of SEQ ID NO:21, 25, 37, or 39.

Also of interest is the complement of such isolated nucleotide fragment.

In a second embodiment, this invention concerns such isolated nucleotide sequence or its complement which comprises at least one motif corresponding substantially to any of the amino acid sequences set forth in SEQ ID NOs:2, 7, 9, 11, 13, 15, 17, 19, 21, 23, 25, 27, 29, 31, 33, 35, 37, 39, 41, 93, 95, 97, or 99 wherein said motif is a conserved subsequence. Examples of such motifs, among others that can be identified, are shown in SEQ ID NOs:80-91. Also of interest is the use of such fragment or a part thereof in antisense inhibition or co-suppression of cytochrome P450 activity in a transformed plant.

In a third embodiment this invention concerns such isolated nucleotide fragment of claim 1 complement thereof wherein the fragment or a part thereof is useful in antisense inhibition or co-suppression of cytochrome P450 activity in a transformed plant.

In a fourth embodiment this invention concerns an isolated nucleotide sequence fragment comprising a nucleic acid sequence encoding a first polypeptide associated with controlling embryo/endosperm size during seed development wherein said polypeptide has an amino acid identity of at least 50%, 55%, 60%, 61%, 65%, 70%, 75%, 77%, 80%, 85%, 90%, 95%, or 100% based on the Clustal method of alignment when compared to a second polypeptide selected from the group consisting of SEQ ID NO:2, 7, 9, 11, 13, 15, 17, 19, 21, 23, 25, 27, 29, 31, 33, 35, 37, 39, 41, 42, 43, 44, 45, 46, 47, 93, 95, 97, or 99. Also of interest is the complement of such sequence.

In a fifth embodiment, this invention concerns this isolated nucleotide sequence of or its complement which comprises at least one motif corresponding substantially to any of the amino acid sequences set forth in SEQ ID NOs:2, 7, 9, 11, 13, 15, 17, 19, 21, 23, 25, 27, 29, 31, 33, 35, 37, 39, 41, 42, 43, 44, 45, 46, 47, 93, 95, 97, or 99, wherein said motif is a conserved subsequence. Any of these fragments or complements or part of either can be useful in antisense inhibition or co-suppression of cytochrome P450 activity in a transformed plant.

In a sixth embodiment, this invention concerns an isolated nucleic acid fragment comprising a promoter wherein said promoter consists essentially of the nucleotide sequence set forth in SEQ ID NOs:3, 4, 104, or 105, or said promoter consists essentially of a fragment or subfragment that is substantially similar and functionally equivalent to the nucleotide sequence set forth in SEQ ID NOs:3, 4, 104, or 105.

In a seventh embodiment, this invention concerns chimeric constructs comprising any of the foregoing nucleic acid fragment or complement thereof or part of either operably linked to at least one regulatory sequence. Also, of interest are plants comprising such chimeric constructs in their genome, plant tissue or cells obtained from such plants, seeds obtained from these plants and oil obtained from such seeds.

In an eighth embodiment, this invention concerns a method of controlling embryolendosperm size during seed development in plants which comprises:

(a) transforming a plant with a chimeric construct of the invention;

(b) growing the transformed plant under conditions suitable for the expression of the chimeric construct; and

(c) selecting those transformed plants which produce seeds having an altered embryo/endosperm size.

In a ninth embodiment, this invention concerns a method to isolate nucleic acid fragments encoding polypeptides associated with controlling embryo/endosperm size during seed development which comprises:

(a) comparing SEQ ID NOs:2, 7, 9, 11, 13, 15, 17, 19, 21, 23, 25, 27, 29, 31, 33, 35, 37, 39, 41, 42, 43, 44, 45, 46, 47, 93, 95, 97, or 99, with other polypeptide sequences associated with controlling embryo/endosperm size during seed development;

(b) identifying the conserved sequences(s) or 4 or more amino acids obtained in step (a);

(c) making region-specific nucleotide probe(s) or oligomer(s) based on the conserved sequences identified in step (b); and

(d) using the nucleotide probe(s) or oligomer(s) of step (c) to isolate sequences associated with controlling embryo/endosperm size during seed development by sequence dependent protocols.

In a tenth embodiment, this invention also concerns a method of mapping genetic variations related to controlling embryo/endosperm size during seed development and/or altering oil phenotypes in plants comprising:

(a) crossing two plant varieties; and

(b) evaluating genetic variations with respect to:

(i) a nucleic acid sequence selected from the group consisting of SEQ ID NO:1, 3, 4, 5, 6, 8, 10, 12, 14, 16, 18, 20, 22, 24, 26, 28, 30, 32, 34, 36, 38, 40, 92, 94, 96, 98, 100, 102, 104, or 105; or

(ii) a nucleic acid sequence encoding a polypeptide selected from the group consisting of SEQ ID NO:2, 7, 9, 11, 13, 15, 17, 19, 21, 23, 25, 27, 29, 31, 33, 35, 37, 39, 41, 42, 43, 44, 45, 46, 47, 80-91, 93, 95, 97, or 99;

in progeny plants resulting from the cross of step (a) wherein the evaluation is made using a method selected from the group consisting of: RFLP analysis, SNP analysis, and PCR-based analysis.

In an eleventh embodiment, this invention concerns a method of molecular breeding to obtain altered embryo/endosperm size during seed development and/or altered oil phenotypes in plants comprising:

(a) crossing two plant varieties; and

(b) evaluating genetic variations with respect to:

(i) a nucleic acid sequence selected from the group consisting of SEQ ID NO:1, 3, 4, 5, 6, 8, 10, 12, 14, 16, 18, 20, 22, 24, 26, 28, 30, 32, 34, 36, 38, 40, 92, 94, 96, 98, 100, 102, 104, or 105; or

(ii) a nucleic acid sequence encoding a polypeptide selected from the group consisting of SEQ ID NO:2, 7, 9, 11, 13, 15, 17, 19, 21, 23, 25, 27, 29, 31, 33, 35, 37, 39, 41, 42, 43, 44, 45, 46, 47, 80-91, 93, 95, 97, or 99;

in progeny plants resulting from the cross of step (a) wherein the evaluation is made using a method selected from the group consisting of: RFLP analysis, SNP analysis, and PCR-based analysis.

### BRIEF DESCRIPTION OF THE FIGURES AND SEQUENCE LISTINGS

The invention can be more fully understood from the following detailed description and the accompanying drawings and Sequence Listing which form a part of this application.

FIG. 1 shows an alignment of the sequence of the GE gene and ge mutant alleles. The allelic mutations resulting in a giant embryo phenotype are noted by a on the complementary strand. Each mutation is labeled and the base change is shown (the corresponding complementary base changes on the coding strand are “*” noted below) and the resulting amino acid change is noted parenthetically (i.e. wild-type→mutant). The ge-1 mutant had a mutation that alters the G at nucleotide 1482 to an A, changing the corresponding Trp residue to a premature translational stop (UGG codon to UGA). In ge-2, the G at nucleotide 1451 was altered to A, again changing the encoded Trp to a premature translational stop (UAG). In ge-3 and ge-9, the C at nucleotide 1177 was altered to T, changing a Pro residue, which is highly conserved among cytochrome P450 proteins, into Ser. In ge-4, the C at nucleotide 1388 was altered to G, changing a Pro residue into Ala. In ge-5, the C at nucleotide 28 was altered to T, causing a premature translational stop (UAA). In ge-6, the A at nucleotide 1067 was altered to C, causing the change of Gin, which is conserved among the CYP78 group, into Pro. In ge-8, we found two mutations: the T at nucleotide 559 was altered to C, causing the change of Ser to Pro, and the C at nucleotide 1328 was altered to T, causing the change of Pro to Leu. One 91 nucleotide-long intron was found between nucleotides 972 and 973.

FIG. 2 shows an alignment of the rice GE (SEQ ID NO:2), barley GE-homolog (SEQ ID NO:93), maize GE1-homolog (SEQ ID NO:95), maize GE2-homolog (SEQ ID NO:97), maize GE3-homolog (SEQ ID NO:99), lily GE-homolog (SEQ ID NO:41), orchid gi 1173624 (SEQ ID NO:43), Arabidopsis gi 1235138 (SEQ ID NO:42), Arabidopsis gi 8920576 (SEQ ID NO:47), columbine GE-homolog (SEQ ID NO:35), soybean GE-homolog (SEQ ID NO:23), Arabidopsis gi 11249511 (SEQ ID NO:44), soybean gi 5921926 (SEQ ID NO:45), soybean GE-homolog (SEQ ID NO:25), soybean GE-homolog (SEQ ID NO:21), and Arabidopsis gi 3831440 (SEQ ID NO:46). The boxed residues are predicted helical regions identified by the Bioscout DSC program (King and Sternberg (1996) Protein Sci 5:2298-2310). Other boxed elements include “SRS” or substrate-recognition-sites which are hypervariable sequences in the cytochrome P450 structure, “PPP” clusters of prolines often Pro-Pro-Gly-Pro in cytochrome P450s, “F-G loop” which is the substrate access channel (part of the conserved sequence motif of SEQ ID NO:83), the conserved “GXDT” the proton transfer groove involved in heme interaction and enzyme catalysis (part of the conserved sequence motif of SEQ ID NO:85), “EXXR” the K-helix motif conserved in all cytochrome P450s necessary for heme stabilization and core structure stability (part of conserved sequence motif of SEQ ID NO:88), and “FXXGXRXCXG” the conserved heme binding site with the cysteine that contacts the heme (part of the conserved sequence motif of SEQ ID NO:90).

Table 1 lists the polypeptides that are described herein, the designation of the genomic or cDNA clones that comprise the nucleic acid fragments encoding polypeptides representing all or a substantial portion of these polypeptides, and the corresponding identifier (SEQ ID NO:) as used in the attached Sequence Listing. The sequence descriptions and Sequence Listing attached hereto comply with the rules governing nucleotide and/or amino acid sequence disclosures in patent applications as set forth in 37 C.F.R. §1.821-1.825.

TABLE 1
Genes Encoding Enzymes Associated With Altering
Embryo/Endosperm Size During Seed Development

| | | SEQ ID NO: | |
|---|---|---|---|
| Cytochrome P450 | | | (Amino |
| Enzymes | Clone Designation | (Nucleotide) | Acid) |
| Rice (Oryza sativa) | bac4d1g.pk001.l12f | 1 | 2 |
| Rice (Oryza sativa) | bac1i1g.pk001.d18 | 3 | |
| Rice (Oryza sativa) | bac4d1g.pk001.o6 | 4 | |
| Rice (Oryza sativa) | bac4d1g.pk001.k21 | 5 | |
| Rice (Oryza sativa) | rca1c.pk007.n11:fis | 6 | 7 |
| Rice (Oryza sativa) | rls2.pk0022.b12:fis | 8 | 9 |
| Rice (Oryza sativa) | rr1.pk0044.e7 | 10 | 11 |
| Maize (Zea mays) | cbn10.pk0034.f8:fis | 12 | 13 |
| Maize (Zea mays) | p0037.crwbn23r | 14 | 15 |
| Maize (Zea mays) | p0121.cfrmn62r:fis | 16 | 17 |
| Maize (Zea mays) | contig of: | 18 | 19 |
| | p0014.ctusi51r | | |
| | p0014.ctutw92r:fis | | |
| | p0022.cglnh53r | | |
| | p0122.ckama19r | | |
| | p9998.cmrne01rb | | |
| Soybean (Glycine max) | sdp2c.pk042.p12:fis | 20 | 21 |
| Soybean (Glycine max) | contig of: se1.20e06 | 22 | 23 |
| | se4.pk0009.e9 | | |
| Soybean (Glycine max) | sfl1.pk0010.a2:fis | 24 | 25 |
| Soybean (Glycine max) | src3c.pk009.k13 | 26 | 27 |
| Sunflower (Helianthus sp.) | hso1c.pk003.n10 | 28 | 29 |
| Sunflower (Helianthus sp.) | hss1c.pk004.b24 | 30 | 31 |
| Wheat (Triticum aestivum) | contig of: | 32 | 33 |
| | wdk2c.pk013.c20 | | |
| | wre1n.pk0056.b6 | | |
| Columbine (Aquilegia | eav1c.pk006.n4:fis | 34 | 35 |
| vulgaris) | | | |
| Grape (Vitis sp.) | veb1c.pk001.k11:fis | 36 | 37 |
| Guayule (Parthenium | epb3c.pk005.d14 | 38 | 39 |
| argentatum Grey) | | | |
| Lily (Astroemeria | eae1s.pk003.b24:fis | 40 | 41 |
| caryophylla) | | | |
| Barley (Hordeum | bdl1c.pk003.h16 | 92 | 93 |
| vulgare) | | | |
| Maize (Zea mays) | p0037.crwbn23r:fis | 94 | 95 |
| Maize (Zea mays) | cbn10.pk0034.f8.f | 96 | 97 |
| Maize (Zea mays) | cpls1s.pk001.m19 | 98 | 99 |

SEQ ID NO:1 and 2 represent the wild-type open-reading-frame (ORF) DNA sequence and the translated amino acid sequence, respectively, for the rice cytochrome P450 gene, which is responsible for the giant embryo phenotype when mutated. SEQ ID NO:3 represents 17 kb of genomic DNA sequence containing the GE ORF (nucleotides 8301 to 9969) which is interrupted by a 91 nucleotide intron (9273 to 9363). SEQ ID NO:4 represents the 8300 nucleotides upstream of the GE ORF that contains the promoter for the gene and the 5′ untranslated (UTR) portion of the GE mRNA. SEQ ID NO:5 represents the 7224 nucleotides downstream of the GE ORF that contains the 3′-UTR and polyadenylation sequences for the gene. There were no other genes, besides GE, detected by BLAST homology that were contained within this 17 kb region of the rice genome. SEQ ID NOs:80-91 are conserved sequence motifs that re useful in identifying cytochrome P450 genes that are functional homologs of GE. SEQ ID NOs:104 and 105 are upstream promoter sequences for maize homologs zmGE1 and zmGE2, respectively (see Example 13 for more detail).

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. §1.822.

### DETAILED DESCRIPTION OF THE INVENTION

As used herein, an “isolated nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The terms “subfragment that is functionally equivalent” and “functionally equivalent subfragment” are used interchangeably herein. These terms refer to a portion or subsequence of an isolated nucleic acid fragment in which the ability to alter gene expression or produce a certain phenotype is retained whether or not the fragment or subfragment encodes an active enzyme. For example, the fragment or subfragment can be used in the design of chimeric constructs to produce the desired phenotype in a transformed plant. Chimeric constructs can be designed for use in co-suppression or antisense by linking a nucleic acid fragment or subfragment thereof, whether or not it encodes an active enzyme, in the appropriate orientation relative to a plant promoter sequence.

The terms “homology”, “homologous”, “substantially similar” and “corresponding substantially” are used interchangeably herein. They refer to nucleic acid fragments wherein changes in one or more nucleotide bases does not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences.

Moreover, the skilled artisan recognizes that substantially similar nucleic acid sequences encompassed by this invention are also defined by their ability to hybridize, under moderately stringent conditions (for example, 1× SSC, 0.1% SDS, 60° C.) with the sequences exemplified herein, or to any portion of the nucleotide sequences reported herein and which are functionally equivalent to the gene or the promoter of the invention. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions involves a series of washes starting with 6× SSC, 0.5% SDS at room temperature for 15 min, then repeated with 2× SSC, 0.5% SDS at 45° C. for 30 min, and then repeated twice with 0.2× SSC, 0.5% SDS at 50° C. for 30 min. A more preferred set of stringent conditions involves the use of higher temperatures in which the washes are identical to those above except for the temperature of the final two 30 min washes in 0.2× SSC, 0.5% SDS was increased to 60° C. Another preferred set of highly stringent conditions involves the use of two final washes in 0.1× SSC, 0.1% SDS at 65° C.

With respect to the degree of substantial similarity between the target (endogenous) mRNA and the RNA region in the construct having homology to the target mRNA, such sequences should be at least 25 nucleotides in length, preferably at least 50 nucleotides in length, more preferably at least 100 nucleotides in length, again more preferably at least 200 nucleotides in length, and most preferably at least 300 nucleotides in length; and should be at least 80% identical, preferably at least 85% identical, more preferably at least 90% identical, and most preferably at least 95% identical.

Sequence alignments and percent similarity calculations may be determined using a variety of comparison methods designed to detect homologous sequences including, but not limited to, the Megalign program of the LASARGENE bioinformatics computing suite (DNASTAR Inc., Madison, Wis.). Multiple alignment of the sequences are performed using the Clustal method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric construct” refers to a combination of nucleic acid fragments that are not normally found together in nature. Accordingly, a chimeric construct may comprise regulatory sequences and coding sequences that are derived from different sources, or regulatory sequences and coding sequences derived from the same source, but arranged in a manner different than that normally found in nature. A “foreign” gene refers to a gene not normally found in the host organism, but that is introduced into the host organism by gene transfer. Foreign genes can comprise native genes inserted into a non-native organism, or chimeric constructs. A “transgene” is a gene that has been introduced into the genome by a transformation procedure.

“Coding sequence” refers to a DNA sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

“Promoter” refers to a DNA sequence capable of controlling the expression of a coding sequence or functional RNA. The promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue-specificity of a promoter. Promoter sequences can also be located within the transcribed portions of genes, and/or downstream of the transcribed sequences. Promoters may be derived in their entirety from a native gene, or be composed of different elements derived from different promoters found in nature, or even comprise synthetic DNA segments. It is understood by those skilled in the art that different promoters may direct the expression of an isolated nucleic acid fragment in different tissues or cell types, or at different stages of development, or in response to different environmental conditions. Promoters which cause an isolated nucleic acid fragment to be expressed in most cell types at most times are commonly referred to as “constitutive promoters”. New promoters of various types useful in plant cells are constantly being discovered; numerous examples may be found in the compilation by Okamuro and Goldberg, (1989) Biochemistry of Plants 15:1-82. It is further recognized that since in most cases the exact boundaries of regulatory sequences have not been completely defined, DNA fragments of some variation may have identical promoter activity.

Specific examples of promoters that may be useful in expressing the nucleic acid fragments of the invention include, but are not limited to, the GE promoter disclosed in this application (SEQ ID NO:4), oleosin promoter (PCT Publication WO99/65479, published on Dec. 12, 1999), maize 27 kD zein promoter (Ueda et al (1994) Mol Cell Bio 14:4350-4359), ubiquitin promoter (Christensen et al (1992) Plant Mol Biol 18:675-680), SAM synthetase promoter (PCT Publication WO00/37662, published on Jun. 29, 2000), or CaMV 35S (Odell et al (1985) Nature 313:810-812).

An “intron” is an intervening sequence in a gene that does not encode a portion of the protein sequence. Thus, such sequences are transcribed into RNA but are then excised and are not translated. The term is also used for the excised RNA sequences. An “exon” is a portion of the sequence of a gene that is transcribed and is found in the mature messenger RNA derived from the gene, but is not necessarily a part of the sequence that encodes the final gene product.

The “translation leader sequence” refers to a DNA sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency.

Examples of translation leader sequences have been described (Turner, R. and Foster, G. D. (1995) Molecular Biotechnology 3:225). The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by lngelbrecht et al., (1989) Plant Cell 1:671-680.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA. “Messenger RNA (mRNA)” refers to the RNA that is without introns and that can be translated into protein by the cell. “CDNA” refers to a DNA that is complementary to and synthesized from a mRNA template using the enzyme reverse transcriptase. The cDNA can be single-stranded or converted into the double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA and that blocks the expression of a target isolated nucleic acid fragment (U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The term “endogenous RNA” refers to any RNA which is encoded by any nucleic acid sequence present in the genome of the host prior to transformation with the recombinant construct of the present invention, whether naturally-occurring or non-naturally occurring, i.e., introduced by recombinant means, mutagenesis, etc.

The term “non-naturally occurring” means artificial, not consistent with what is normally found in nature.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the invention can be operably linked, either directly or indirectly, 5′ to the target mRNA, or 3′ to the target mRNA, or within the target mRNA, or a first complementary region is 5′ and its complement is 3′ to the target mRNA.

The term “expression”, as used herein, refers to the production of a functional end-product. Expression of an isolated nucleic acid fragment involves transcription of the isolated nucleic acid fragment and translation of the mRNA into a precursor or mature protein. “Antisense inhibition” refers to the production of antisense RNA transcripts capable of suppressing the expression of the target protein. “Co-suppression” refers to the production of sense RNA transcripts capable of suppressing the expression of identical or substantially similar foreign or endogenous genes (U.S. Pat. No. 5,231,020).

“Mature” protein refers to a post-translationally processed polypeptide; i.e., one from which any pre- or propeptides present in the primary translation product have been removed. “Precursor” protein refers to the primary product of translation of mRNA; i.e., with pre- and propeptides still present. Pre- and propeptides may be but are not limited to intracellular localization signals.

“Stable transformation” refers to the transfer of a nucleic acid fragment into a genome of a host organism, including both nuclear and organellar genomes, resulting in genetically stable inheritance. In contrast, “transient transformation” refers to the transfer of a nucleic acid fragment into the nucleus, or DNA-containing organelle, of a host organism resulting in gene expression without integration or stable inheritance. Host organisms containing the transformed nucleic acid fragments are referred to as “transgenic” organisms. The preferred method of cell transformation of rice, corn and other monocots is the use of particle-accelerated or “gene gun” transformation technology (Klein et al., (1987) Nature (London) 327:70-73; U.S. Pat. No. 4,945,050), or an Agrobacterium-mediated method using an appropriate Ti plasmid containing the transgene (Ishida Y. et al., 1996, Nature Biotech. 14:745-750). The term “transformation” as used herein refers to both stable transformation and transient transformation.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

The term “recombinant” refers to an artificial combination of two otherwise separated segments of sequence, e.g., by chemical synthesis or by the manipulation of isolated segments of nucleic acids by genetic engineering techniques.

“PCR” or “Polymerase Chain Reaction” is a technique for the synthesis of large quantities of specific DNA segments, consists of a series of repetitive cycles (Perkin Elmer Cetus Instruments, Norwalk, Conn.). Typically, the double stranded DNA is heat denatured, the two primers complementary to the 3′ boundaries of the target segment are annealed at low temperature and then extended at an intermediate temperature. One set of these three consecutive steps is referred to as a cycle.

Polymerase chain reaction (“PCR”) is a powerful technique used to amplify DNA millions of fold, by repeated replication of a template, in a short period of time. (Mullis et al, Cold Spring Harbor Symp. Quant. Biol. 51:263-273 (1986); Erlich et al, European Patent Application 50,424; European Patent Application 84,796; European Patent Application 258,017, European Patent Application 237,362; Mullis, European Patent Application 201,184, Mullis et al U.S. Pat. No. 4,683,202; Erlich, U.S. Pat. No. 4,582,788; and Saiki et al, U.S. Pat. No. 4,683,194). The process utilizes sets of specific in vitro synthesized oligonucleotides to prime DNA synthesis. The design of the primers is dependent upon the sequences of DNA that are desired to be analyzed. The technique is carried out through many cycles (usually 20-50) of melting the template at high temperature, allowing the primers to anneal to complementary sequences within the template and then replicating the template with DNA polymerase.

The products of PCR reactions are analyzed by separation in agarose gels followed by ethidium bromide staining and visualization with UV transillumination. Alternatively, radioactive dNTPs can be added to the PCR in order to incorporate label into the products. In this case the products of PCR are visualized by exposure of the gel to x-ray film. The added advantage of radiolabeling PCR products is that the levels of individual amplification products can be quantitated.

The terms “recombinant construct”, “expression construct” and “recombinant expression construct” are used interchangeably herein. These terms refer to a functional unit of genetic material that can be inserted into the genome of a cell using standard methodology well known to one skilled in the art. Such construct may be itself or may be used in conjunction with a vector. If a vector is used then the choice of vector is dependent upon the method that will be used to transform host plants as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the invention. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, Western analysis of protein expression, or phenotypic analysis.

Co-suppression constructs in plants previously have been designed by focusing on overexpression of a nucleic acid sequence having homology to an endogenous mRNA, in the sense orientation, which results in the reduction of all RNA having homology to the overexpressed sequence (see Vaucheret et al. (1998) Plant J 16:651-659; and Gura (2000) Nature 404:804-808). The overall efficiency of this phenomenon is low, and the extent of the RNA reduction is widely variable. Recent work has described the use of “hairpin” structures that incorporate all, or part, of an mRNA encoding sequence in a complementary orientation that results in a potential “stem-loop” structure for the expressed RNA (PCT Publication WO 99/53050 published on Oct. 21, 1999). This increases the frequency of co-suppression in the recovered transgenic plants. Another variation describes the use of plant viral sequences to direct the suppression, or “silencing”, of proximal mRNA encoding sequences (PCT Publication WO 98/36083 published on Aug. 20, 1998). Both of these co-suppressing phenomena have not been elucidated mechanistically, although recent genetic evidence has begun to unravel this complex situation (Elmayan et al. (1998) Plant Cell 10:1747-1757).

Plant cytochrome P450 enzymes are NADPH-dependent monooxygenases that are responsible for the oxidative metabolism of a variety of compounds in plants. The cytochrome P450s contain iron-sulfur ligands, termed haem-thiolate complexes, that are responsible for a distinctive absorption spectrum with a maximum at 450 nm in the presence of carbon monoxide. In animal systems P450 enzymes are responsible for detoxification pathways in the liver, inactivation and activation of certain carcinogenic compounds, and drug and hormone metabolism. In plants, the cytochrome P450 family is responsible for, but not limited to, herbicide metabolism, secondary metabolism, and wounding responses.

Surprisingly, it has been found that a single mutation of a cytochrome P450 gene in rice can lead to an alteration of embryolendosperm size during seed development. This gene is named Giant Embryo (GE). Inhibition of the function of the gene leads to enlargement of embryonic tissue at the expense of part of the endosperm tissue. Thus, the GE gene and protein product can regulate proliferation both negatively and positively depending on the tissue. Enlargement of the embryo will result in seeds with high content of valuable components such as oils. A search of GenBank with the rice GE sequence uncovers a number of genes from plants that appear to be homologous.

“Giant embryo-like cytochrome P450” polypeptides would encompass those enzymes from other plants that share sequence and/or functional similarity to the rice GE polypeptide. It is believed that such a polypeptide would comprise a subset of the cytochrome P450 family, and that alteration in the expression of this member would affect embryo-size.

“Motifs” or “subsequences” refer to short regions of conserved sequences of nucleic acids or amino acids that comprise part of a longer sequence. For example, it is expected that such conserved subsequences (for example SEQ ID NOs:80-91) would be important for function, and could be used to identify new homologues of GE-like cytochrome P450s in plants. It is expected that some or all of the elements may be found in a GE-homologue. Also, it is expected that one or two of the conserved amino acids in any given motif may differ in a true GE-homologue.

Thus, in one aspect, this invention concerns an isolated nucleotide fragment comprising a nucleic acid sequence selected from the group consisting of:

(a) a nucleic acid sequence encoding a cytochrome P450 polypeptide associated with controlling embryolendosperm size during seed development having an amino acid identity of at least 61% based on the Clustal method of alignment when compared to a second polypeptide selected from the group consisting of SEQ ID NO:2, 7, 11, 19, 27, or 33; or

(b) a nucleic acid sequence encoding a cytochrome P450 polypeptide associated with controlling embryolendosperm size during seed development having an amino acid identity of at least 65% based on the Clustal method of alignment when compared to a third polypeptide selected from the group consisting of SEQ ID NOs:15, 17, 31, 93, 95, 97, or 99; or

(c) a nucleic acid sequence encoding a cytochrome P450 polypeptide associated with controlling embryo/endosperm size during seed development having an amino acid identity of at least 70% based on the Clustal method of alignment when compared to a third polypeptide selected from the group consisting of SEQ ID NOs:9, 13, 23, 29, 35, or 41; or

(d) a nucleic acid sequence encoding a cytochrome P450 polypeptide associated with controlling embryo/endosperm size during seed development having an amino acid identity of at least 77% based on the Clustal method of alignment when compared to a second polypeptide selected from the group consisting of SEQ ID NOs:21, 25, 37, or 39.

It is well understood by one skilled in the art that many levels of sequence identity are useful in identifying related polypeptide sequences. Useful examples of percent identities are 50%, 55%, 60%, 65%, 70%, 75%, 80%, 85%, 90%, or 95%, or any integer percentage from 55% to 100%.

Also, of interest is the complement of this isolated nucleotide fragment.

The isolated nucleotide sequence or its complement can also comprise at least one, two, three, four, five, six, seven, eight, nine, ten, or eleven motif(s) corresponding substantially to any of the amino acid sequences set forth in SEQ ID NOs:80-91 wherein said motif is a conserved subsequence. In another aspect, this isolated nucleotide fragment or its complement (whether they comprise the aforementioned motif or not) or a part of the fragment or its complement can be used in antisense inhibition or co-suppression of cytochrome P450 activity in a transformed plant. It is appreciated that further embodiments would include at least one, two, three, four, five, six, seven, eight, nine, ten, or eleven motif(s) corresponding substantially to any of the amino acid sequences set forth in SEQ ID NOs:80-91 being used to identify cytochrome P450 polypeptides associated with controlling embryo/endosperm size during seed development.

Protocols for antisense inhibition or co-suppression are well known to those skilled in the art and are described above.

In still a further aspect, this invention concerns an isolated nucleic acid fragment comprising a promoter wherein said promoter consists essentially of the nucleotide sequence set forth in SEQ ID NOs:3, 4, 104, or 105, or said promoter consists essentially of a fragment or subfragment that is substantially similar and functionally equivalent to the nucleotide sequence set forth in SEQ ID NOs:3, 4, 104, or 105.

Also of interest are chimeric constructs comprising any of the above-identified isolated nucleic acid fragments or complements thereof or parts of such fragments or complements operably linked to at least one regulatory sequence.

Plants, plant tissue or plant cells comprising such chimeric constructs in their genome are also within the scope of this invention. Transformation methods are well known to those skilled in the art and are described above. Any plant, dicot or monocot can be transformed with such chimeric constructs.

Examples of monocots include, but are not limited to, corn, wheat, rice, sorghum, millet, barley, palm, lily, Alstroemeria, rye, and oat. Examples of dicots include, but are not limited to, soybean, rape, sunflower, canola, grape, guayule, columbine, cotton, tobacco, peas, beans, flax, safflower, alfalfa.

Plant tissue includes differentiated and undifferentiated tissues or plants, including but not limited to, roots, stems, shoots, leaves, pollen, seeds, tumor tissue, and various forms of cells and culture such as single cells, protoplasm, embryos, and callus tissue. The plant tissue may in plant or in organ, tissue or cell culture.

Also within the scope of this invention are seeds obtained from such plants and oil obtained from these seeds.

In another aspect, this invention concerns a method of controlling embryo/endosperm size during seed development in plants which comprises:

(a) transforming a plant with a chimeric construct of the invention;

(b) growing the transformed plant under conditions suitable for the expression of the chimeric construct; and

(c) selecting those transformed plants which produce seeds having an altered embryo/endosperm size.

The regeneration, development, and cultivation of plants from single plant protoplast transformants or from various transformed explants is well known in the art (Weissbach and Weissbach, In: Methods for Plant Molecular Biology, (Eds.), Academic Press, Inc. San Diego, Calif., (1988)). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous isolated nucleic acid fragment that encodes a protein of interest is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide is cultivated using methods well known to one skilled in the art.

There are a variety of methods for the regeneration of plants from plant tissue.

The particular method of regeneration will depend on the starting plant tissue and the particular plant species to be regenerated.

Methods for transforming dicots, primarily by use of Agrobacterium tumefaciens, and obtaining transgenic plants have been published for cotton (U.S. Pat. No. 5,004,863, U.S. Pat. No. 5,159,135, U.S. Pat. No. 5,518, 908); soybean (U.S. Pat. No. 5,569,834, U.S. Pat. No. 5,416,011, McCabe et. al., BiolTechnology 6:923 (1988), Christou et al., Plant Physiol. 87:671-674 (1988)); Brassica (U.S. Pat. No. 5,463,174); peanut (Cheng et al., Plant Cel/Rep. 15:653-657 (1996), McKently et al., Plant Cell Rep. 14:699-703 (1995)); papaya; and pea (Grant et al., Plant Cell Rep. 15:254-258, (1995)).

Transformation of monocotyledons using electroporation, particle bombardment, and Agrobacterium have also been reported. Transformation and plant regeneration have been achieved in asparagus (Bytebier et al., Proc. Natl. Acad. Sci. (USA) 84:5354, (1987)); barley (Wan and Lemaux, Plant Physiol 104:37 (1994)); Zea mays (Rhodes et al., Science 240:204 (1988), Gordon-Kamm et al., Plant Cell 2:603-618 (1990), Fromm et al., BiolTechnology 8:833 (1990), Koziel et al., BiolTechnology 11: 194, (1993), Armstrong et al., Crop Science 35:550-557 (1995)); oat (Somers et al., BiolTechnology 10: 15 89 (1992)); orchard grass (Horn et al., Plant Cell Rep. 7:469 (1988)); rice (Toriyama et al., TheorAppl. Genet. 205:34, (1986); Part et al., Plant Mol. Biol. 32:1135-1148, (1996); Abedinia et al., Aust. J. Plant Physiol. 24:133-141 (1997); Zhang and Wu, Theor. Appl. Genet. 76:835 (1988); Zhang et al. Plant Cell Rep. 7:379, (1988); Battraw and Hall, Plant Sci. 86:191-202 (1992); Christou et al., Bio/Technology 9:957 (1991)); rye (De la Pena et al., Nature 325:274 (1987)); sugarcane (Bower and Birch, Plant J. 2:409 (1992)); tall fescue (Wang et al., Bio/Technology 10:691 (1992)), and wheat (Vasil et al., Bio/Technology 10:667 (1992); U.S. Pat. No. 5,631,152).

Assays for gene expression based on the transient expression of cloned nucleic acid constructs have been developed by introducing the nucleic acid molecules into plant cells by polyethylene glycol treatment, electroporation, or particle bombardment (Marcofte et al., Nature 335:454-457 (1988); Marcotte et al., Plant Cell 1:523-532 (1989); McCarty et al., Cell 66:895-905 (1991); Hattori et al., Genes Dev. 6:609-618 (1992); Goff et al., EMBO J. 9:2517-2522 (1990)).

Transient expression systems may be used to functionally dissect isolated nucleic acid fragment constructs (see generally, Maliga et al., Methods in Plant Molecular Biology, Cold Spring Harbor Press (1995)). It is understood that any of the nucleic acid molecules of the present invention can be introduced into a plant cell in a permanent or transient manner in combination with other genetic elements such as vectors, promoters, enhancers etc.

In addition to the above discussed procedures, practitioners are familiar with the standard resource materials which describe specific conditions and procedures for the construction, manipulation and isolation of macromolecules (e.g., DNA molecules, plasmids, etc.), generation of recombinant organisms and the screening and isolating of clones, (see for example, Sambrook et al., Molecular Cloning: A Laboratory Manual, Cold Spring Harbor Press (1989); Maliga et al., Methods in Plant Molecular Biology, Cold Spring Harbor Press (1995); Birren et al., Genome Analysis: Detecting Genes, 1, Cold Spring Harbor, N.Y. (1998); Birren et al., Genome Analysis: Analyzing DNA, 2, Cold Spring Harbor, N.Y. (1998); Plant Molecular Biology: A Laboratory Manual, eds. Clark, Springer, New York (1997)).

In a still further aspect this invention concerns a method to isolate nucleic acid fragments encoding polypeptides associated with controlling embryolendosperm size during seed development which comprises:

(a) comparing SEQ ID NOs:2, 7, 9, 11, 13, 15, 17, 19, 21, 23, 25, 27, 29, 31, 33, 35, 37, 39, 41, 42, 43, 44, 45, 46, 47, 93, 95, 97, or 99, with other polypeptide sequences associated with controlling embryo/endosperm size during seed development;

(b) identifying the conserved sequences(s) or 4 or more amino acids obtained in step (a);

(c) making region-specific nucleotide probe(s) or oligomer(s) based on the conserved sequences identified in step (b); and

(d) using the nucleotide probe(s) or oligomer(s) of step (c) to isolate sequences associated with controlling embryo/endosperm size during seed development by sequence dependent protocols.

Examples of conserved sequence elements that would be useful in identifying other plant sequences associated with controlling embryo/endosperm size during seed development can be found in the group comprising, but not limited to, the nucleotides encoding the polypeptides of SEQ ID NO:80, 81, 82, 83, 84, 85, 86, 87, 88, 89, 90, or 91.

In another aspect, this invention also concerns a method of mapping genetic variations related to controlling embryo/endosperm size during seed development and/or altering oil phenotypes in plants comprising:

(a) crossing two plant varieties; and

(b) evaluating genetic variations with respect to:

(i) a nucleic acid sequence selected from the group consisting of SEQ ID NO:1, 3, 4, 5,6, 8, 10, 12, 14, 16, 18, 20, 22, 24, 26, 28, 30, 32, 34, 36, 38, 40, 92, 94, 96, 98,100,102,104, or 105; or

(ii) a nucleic acid sequence encoding a polypeptide selected from the group consisting of SEQ ID NO:2, 7, 9, 11, 13, 15, 17,19, 21, 23, 25, 27, 29, 31, 33, 35, 37, 39, 41, 42, 43, 44, 45, 46, 47, 80-91, 93, 95, 97, or 99;

in progeny plants resulting from the cross of step (a) wherein the evaluation is made using a method selected from the group consisting of: RFLP analysis, SNP analysis, and PCR-based analysis.

In another embodiment, this invention concerns a method of molecular breeding to obtain altered embryo/endosperm size during seed development and/or altered oil phenotypes in plants comprising:

(a) crossing two plant varieties; and

(b) evaluating genetic variations with respect to:

(i) a nucleic acid sequence selected from the group consisting of SEQ ID NO:1, 3, 4, 5, 6, 8, 10, 12, 14, 16, 18, 20, 22, 24, 26, 28, 30, 32, 34, 36, 38, 40, 92, 94, 96, 98, 100, 102, 104, or 105; or

(ii) a nucleic acid sequence encoding a polypeptide selected from the group consisting of SEQ ID NO:2, 7, 9, 11, 13, 15, 17, 19, 21, 23, 25, 27, 29, 31, 33, 35, 37, 39, 41, 42, 43, 44, 45, 46, 47, 80-91, 93, 95, 97, or 99;

in progeny plants resulting from the cross of step (a) wherein the evaluation is made using a method selected from the group consisting of: RFLP analysis, SNP analysis, and PCR-based analysis.

The terms “mapping genetic variation” or “mapping genetic variability” are used interchangeably and define the process of identifying changes in DNA sequence, whether from natural or induced causes, within a genetic region that differentiates between different plant lines, cultivars, varieties, families, or species. The genetic variability at a particular locus (gene) due to even minor base changes can alter the pattern of restriction enzyme digestion fragments that can be generated. Pathogenic alterations to the genotype can be due to deletions or insertions within the gene being analyzed or even single nucleotide substitutions that can create or delete a restriction enzyme recognition site. RFLP analysis takes advantage of this and utilizes Southern blotting with a probe corresponding to the isolated nucleic acid fragment of interest.

Thus, if a polymorphism (i.e., a commonly occurring variation in a gene or segment of DNA; also, the existence of several forms of a gene (alleles) in the same species) creates or destroys a restriction endonuclease cleavage site, or if it results in the loss or insertion of DNA (e.g., a variable nucleotide tandem repeat (VNTR) polymorphism), it will alter the size or profile of the DNA fragments that are generated by digestion with that restriction endonuclease. As such, individuals that possess a variant sequence can be distinguished from those having the original sequence by restriction fragment analysis. Polymorphisms that can be identified in this manner are termed “restriction fragment length polymorphisms: (“RFLPs”). RFLPs have been widely used in human and plant genetic analyses (Glassberg, UK Patent Application 2135774; Skolnick et al, Cytogen. Cell Genet. 32:58-67 (1982); Botstein et al, Ann. J. Hum. Genet. 32:314-331 (1980); Fischer et al (PCT Application WO 90/13668; Uhlen, PCT Application WO 90/11369).

A central attribute of “single nucleotide polymorphisms” or “SNPs” is that the site of the polymorphism is at a single nucleotide. SNPs have certain reported advantages over RFLPs or VNTRs. First, SNPs are more stable than other classes of polymorphisms. Their spontaneous mutation rate is approximately 10-9 (Kornberg, DNA Replication, W. H. Freeman & Co., San Francisco, 1980), approximately, 1,000 times less frequent than VNTRs (U.S. Pat. No. 5,679,524). Second, SNPs occur at greater frequency, and with greater uniformity than RFLPs and VNTRs. As SNPs result from sequence variation, new polymorphisms can be identified by sequencing random genomic or cDNA molecules. SNPs can also result from deletions, point mutations and insertions. Any single base alteration, whatever the cause, can be a SNP. The greater frequency of SNPs means that they can be more readily identified than the other classes of polymorphisms.

SNPs can be characterized using any of a variety of methods. Such methods include the direct or indirect sequencing of the site, the use of restriction enzymes where the respective alleles of the site create or destroy a restriction site, the use of allele-specific hybridization probes, the use of antibodies that are specific for the proteins encoded by the different alleles of the polymorphism or by other biochemical interpretation. SNPs can be sequenced by a number of methods. Two basic methods may be used for DNA sequencing, the chain termination method of Sanger et al, Proc. Nati. Acad. Sci. (U.S.A.) 74:5463-5467 (1977), and the chemical degradation method of Maxam and Gilbert, Proc. Natl. Acad. Sci. (U.S.A.) 74: 560-564 (1977).

Furthermore, single point mutations can be detected by modified PCR techniques such as the ligase chain reaction (“LCR”) and PCR-single strand conformational polymorphisms (“PCR-SSCP”) analysis. The PCR technique can also be used to identify the level of expression of genes in extremely small samples of material, e.g., tissues or cells from a body. The technique is termed reverse transcription-PCR (“RT-PCR”).

The term “molecular breeding” defines the process of tracking molecular markers during the breeding process. It is common for the molecular markers to be linked to phenotypic traits that are desirable. By following the segregation of the molecular marker or genetic trait, instead of scoring for a phenotype, the breeding process can be accelerated by growing fewer plants and eliminating assaying or visual inspection for phenotypic variation. The molecular markers useful in this process include, but are not limited to, any marker useful in identifying mapable genetic variations previously mentioned, as well as any closely linked genes that display synteny across plant species. The term “synteny” refers to the conservation of gene placement/order on chromosomes between different organisms. This means that two or more genetic loci, that may or may not be closely linked, are found on the same chromosome among different species. Another term for synteny is “genome colinearity”.

### EXAMPLES

The present invention is further defined in the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

The disclosure of each reference set forth herein is incorporated herein by reference in its entirety.

### Example 1

### Composition of cDNA Libraries; Isolation and Sequencing of cDNA Clones

cDNA libraries representing mRNAs from various rice, columbine, grape, guayule, Peruvian lily, corn, soybean, sunflower, and wheat tissues were prepared as described below. The characteristics of the libraries are described below in Table 2.

TABLE 2
Genomic and cDNA Libraries from Rice, Columbine,
Grape, Guayule, Peruvian lily, Corn, Soybean,
Sunflower, and Wheat

| Library | Tissue | Clone |
|---|---|---|
| bac1i1g | The BAC clone, 1l, is derived from the | bac1i1g.pk001.d18 |
| | Texas A & M library. The insert is | |
| | 100 kb long. This BAC clone | |
| | covers the Giant Embryo region. | |
| | The average insertion length of | |
| | this library is 1-2 kb. | |
| bac4dlg | The BAC clone, 4D, is derived from the | bac4d1g.pk001.o6 |
| | Texas A & M library. The insert is 80 kb | bac4d1g.pk001.k21 |
| | long. This BAC clone covers part of | bac4d1g.pk001.l12.f |
| | the Giant Embryo region. The | |
| | average insertion length of this library | |
| | is 1-2 kb. | |
| baclilg | The BAC clone 1l is derived from the | bac1i1g.pk001.p23 |
| | Texas A & M library. The insert is | |
| | 100 kb long. This BAC clone covers the | |
| | Giant Embryo region. The average | |
| | insertion length of this library | |
| | is 1-2 kb. | |
| Bacm | Maize BAC fingerprinting | bacm.pk015.d18.f |
| | | bacm.pk019.j23 |
| bdl1c | Barley (Hordeum vulgaris) leaf | bdl1c.pk003.h16 |
| | tissues infected with M grisea | |
| | (6043) for 48 hours | |
| eav1c | Columbine (Aquilegia vulgaris) | eav1c.pk006.n4:fis |
| | developing seeds (looking for delta | |
| | 5 desaturase genes) | |
| veb1c | Grape (Vitis sp.) early berries | veb1c.pk001.k11:fis |
| epb3c | Guayule (Parthenium argentatum, | |
| | 11591) stem | |
| | bark harvested at 12/28/93- high | epb3c.pk005.d14 |
| | activity for rubber biosynthesis | |
| eae1s | Alstroemeria cayophylla emerging | eae1s.pk003.b24:fis |
| | leaf from mature stem | |
| cbn10 | Corn Developing Kernel (Embryo | cbn10.pk0034.f8:fis |
| | and Endosperm); 10 Days After | |
| | Pollination | |
| cpe1c | Corn (Zea mays L.) pooled | cpe1c.pk011.m11 |
| | BMS treated with chemicals | |
| | related to phosphatase | |
| cpf1c | Corn (Zea mays L.) pooled | cpf1c.pk001.c2 |
| | BMS treated with chemicals related | |
| | to protein synthesis | |
| cpj1c | Corn (Zea mays L.) pooled | cpj1c.pk002.d2 |
| | BMS treated with chemicals related | |
| | to membrane ionic force | |
| cpls1s | Maize, leaf sheath, pulvinus region. | cpls1s.pk001.m19 |
| | Identify genes that are expressed | |
| | in the pulvinus region of the leaf | |
| | sheath | |
| p0022 | Green leaves treated with JA 24 hr | p0022.cglnh53rb |
| | before collection [JA] = | |
| | 1 mg/ml in 0.02% Tween 20 | |
| | middle ¾ of | |
| | the 3rd leaf blade and mid rib only | |
| | (normalized P0012) | |
| p0037 | corn Root Worm infested V5 roots | p0037.crwbn23r |
| p0083 | 7 DAP whole kernels | p0083.cldaq05r |
| | | p0083.cldaq05ra |
| p0121 | shank tissue collected from ears | p0121.cfrmn62r:fis |
| | 5DAP, Screened 1 | |
| p9998 | Clone confirmations that did not | p9998.cmrne01rb |
| | match expected clone | |
| rca1c | Rice Nipponbare Callus. | rca1c.pk007.n11:fis |
| rls2 | Rice Leaf 15 Days After Germination, | rls2.pk0022.b12:fis |
| | 2 Hours After Infection of Strain | |
| | Magnaporthe grisea 4360-R-67 | |
| | (AVR2-YAMO); Susceptible | |
| rr1 | Rice Root of Two Week Old Developing | rr1.pk0044.e7 |
| | Seedling | |
| sdp2c | Soybean (Glycine max L.) | sdp2c.pk042.p12:fis |
| | developing pods 6-7 mm | |
| se4 | Soybean Embryo, 19 Days After | se4.pk0009.e9 |
| | Flowering | |
| sf11 | Soybean Immature Flower | sfl1.pk0010.a2:fis |
| src3c | Soybean 8 Day Old Root Infected | src3c.pk009.k13 |
| | With Cyst Nematode | |
| hso1c | oxalate oxidase-transgenic sunflower | hso1c.pk003.n10 |
| | plants | |
| hss1c | Sclerotinia infected sunflower | hss1c.pk004.b24 |
| | plants, purpose isolation of full | |
| | length Sclerotinia induced cDNAs | |
| wdk2c | Wheat Developing Kernel, 7 Days | wdk2c.pk013.c20 |
| | After Anthesis. | |

cDNA libraries may be prepared by any one of many methods available. For example, the cDNAs may be introduced into plasmid vectors by first preparing the cDNA libraries in Uni-ZAP™ XR vectors according to the manufacturer's protocol (Stratagene Cloning Systems, La Jolla, Calif.). The Uni-ZAP™ XR libraries are converted into plasmid libraries according to the protocol provided by Stratagene. Upon conversion, cDNA inserts will be contained in the plasmid vector pBluescript. In addition, the cDNAs may be introduced directly into precut Bluescript II SK(+) vectors (Stratagene) using T4 DNA ligase (New England Biolabs), followed by transfection into DH10B cells according to the manufacturer's protocol (GIBCO BRL Products). Once the cDNA inserts are in plasmid vectors, plasmid DNAs are prepared from randomly picked bacterial colonies containing recombinant pBluescript plasmids, or the insert cDNA sequences are amplified via polymerase chain reaction using primers specific for vector sequences flanking the inserted cDNA sequences. Amplified insert DNAs or plasmid DNAs are sequenced in dye-primer sequencing reactions to generate partial cDNA sequences (expressed sequence tags or “ESTs”; see Adams et al., (1991) Science 252:1651-1656). The resulting ESTs are analyzed using a Perkin Elmer Model 377 fluorescent sequencer.

Full-insert sequence (FIS) data is generated utilizing a modified transposition protocol. Clones identified for FIS are recovered from archived glycerol stocks as single colonies, and plasmid DNAs are isolated via alkaline lysis. Isolated DNA templates are reacted with vector primed M13 forward and reverse oligonucleotides in a PCR-based sequencing reaction and loaded onto automated sequencers. Confirmation of clone identification is performed by sequence alignment to the original EST sequence from which the FIS request is made.

Confirmed templates are transposed via the Primer Island transposition kit (PE Applied Biosystems, Foster City, Calif.) which is based upon the Saccharomyces cerevisiae Tyl transposable element (Devine and Boeke (1994) Nucleic Acids Res. 22:3765-3772). The in vitro transposition system places unique binding sites randomly throughout a population of large DNA molecules. The transposed DNA is then used to transform DH10B electro-competent cells (Gibco BRL/Life Technologies, Rockville, Md.) via electroporation. The transposable element contains an additional selectable marker (named DHFR; Fling and Richards (1983) Nucleic Acids Res. 11:5147-5158), allowing for dual selection on agar plates of only those subclones containing the integrated transposon. Multiple subclones are randomly selected from each transposition reaction, plasmid DNAs are prepared via alkaline lysis, and templates are sequenced (ABI Prism dye-terminator ReadyReaction mix) outward from the transposition event site, utilizing unique primers specific to the binding sites within the transposon.

Sequence data is collected (ABI Prism Collections) and assembled using Phred/Phrap (P. Green, University of Washington, Seattle). Phred/Phrap is a public domain software program which re-reads the ABI sequence data, re-calls the bases, assigns quality values, and writes the base calls and quality values into editable output files. The Phrap sequence assembly program uses these quality values to increase the accuracy of the assembled sequence contigs. Assemblies are viewed by the Consed sequence editor (D. Gordon, University of Washington, Seattle).

### Example 2

### Identification of cDNA Clones

Clones for cDNAs encoding GE-like cytochrome P450 proteins were identified by conducting BLAST searches. (Basic Local Alignment Search Tool; Altschul et al. (1993) J. Mol. Biol. 215:403-410) searches for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences obtained in Example 1 were analyzed for similarity to all publicly available DNA sequences contained in the “nr” database using the BLASTN algorithm provided by the National Center for Biotechnology Information (NCBI). The DNA sequences were translated in all reading frames and compared for similarity to all publicly available protein sequences contained in the “nr” database using the BLASTX algorithm (Gish and States (1993) Nat. Genet. 3:266-272) provided by the NCBI. For convenience, the P-value (probability) of observing a match of a cDNA sequence to a sequence contained in the searched databases merely by chance as calculated by BLAST are reported herein as “pLog” values, which represent the negative of the logarithm of the reported P-value. Accordingly, the greater the pLog value, the greater the likelihood that the cDNA sequence and the BLAST “hit” represent homologous proteins.

ESTs submitted for analysis are compared to the genbank database as described above. ESTs that contain sequences more 5- or 3-prime can be found by using the BLASTn algorithm (Altschul et al (1997) Nucleic Acids Res. 25:3389-3402.) against the Du Pont proprietary database comparing nucleotide sequences that share common or overlapping regions of sequence homology. Where common or overlapping sequences exist between two or more nucleic acid fragments, the sequences can be assembled into a single contiguous nucleotide sequence, thus extending the original fragment in either the 5 or 3 prime direction. Once the most 5-prime EST is identified, its complete sequence can be determined by Full Insert Sequencing as described in Example 1. Homologous genes belonging to different species can be found by comparing the amino acid sequence of a known gene (from either a proprietary source or a public database) against an EST database using the tBLASTn algorithm. The tBLASTn algorithm searches an amino acid query against a nucleotide database that is translated in all 6 reading frames. This search allows for differences in nucleotide codon usage between different species, and for codon degeneracy.

### Example 3

### Characterization of cDNA Clones Encoding GE-Like Cytochrome P450 Proteins

The BLASTX search using the EST sequences from clones listed in Table 3 revealed similarity of the polypeptides encoded by the cDNAs to cytochrome P450 proteins from Arabidopsis [Arabidopsis thaliana] (NCBI General Identifier Nos. gi, [SEQ ID NO:42] which is identical to gi 12325138 and gi 15221132; and gi 11249511, [SEQ ID NO:44]; and gi 3831440, [SEQ ID NO:46]; and gi 8920576, [SEQ ID NO:47]), and a cytochrome P450 protein from orchid [Phalaenopsis sp.SM9108] (NCBI General Identifier No. gi 1173624, [SEQ ID NO:43]), and a cytochrome P450 protein from soybean [Glycine max] (NCBI General Identifier No. gi 5921926, [SEQ ID NO:45]). Shown in Table 3 are the BLAST results for individual ESTs (“EST”), the sequences of the entire cDNA inserts comprising the indicated cDNA clones (“FIS”), the sequences of contigs assembled from two or more ESTs (“Contig”), sequences of contigs assembled from an FIS and one or more ESTs (“Contig*”), or sequences encoding an entire protein derived from an FIS, a contig, or an FIS and PCR (“CGS”):

TABLE 3
BLAST Results for Sequences Encoding the
Rice Giant Embryo Cytochrome P450
and Polypeptides Homologous To GE

| | | BLAST pLog Score | | | | | |
|---|---|---|---|---|---|---|---|
| Clone | Status | 7109461 | 1173624 | 11249511 | 5921926 | 3831440 | 8920576 |
| bac4d1g.pk001.l12.fis | CGS | 155.0 | | | | | |
| rca1c.pk007.n11:fis | FIS | | 24.0 | | | | |
| rls2.pk0022.b12:fis | FIS | | | 78.3 | | | |
| rr1.pk0044.e7 | EST | | | | 3.5 | | |
| cbn10.pk0034.f8:fis | FIS | | 114.0 | | | | |
| p0037.crwbn23r | EST | | 63.2 | | | | |
| p0121.cfrmn62r:fis | FIS | | 156.0 | | | | |
| Contig of: | CON | | | | | 126.0 | |
| p0014.ctusi51r | | | | | | | |
| p0014.ctutw92r:fis | | | | | | | |
| p0022.cglnh53r | | | | | | | |
| p0122.ckama19r | | | | | | | |
| p9998.cmrne01rb | | | | | | | |
| sdp2c.pk042.p12:fis | FIS | | | | 180.0 | | |
| Contig of: | CON | | | | | | 180.0 |
| se1.20e06 | | | | | | | |
| se4.pk0009.e9 | | | | | | | |
| sfl1.pk0010.a2:fis | FIS | | | | 180.0 | | |
| src3c.pk009.k13 | EST | | 32.5 | | | | |
| hso1c.pk003.n10 | EST | 58.1 | | | | | |
| hss1c.pk004.b24 | EST | | | | | | 42.0 |
| contig of: | CON | | | | 27.7 | | |
| wdk2c.pk013.c20 | | | | | | | |
| wre1n.pk0056.b6 | | | | | | | |
| eav1c.pk006.n4:fis | FIS | 180.0 | | | | | |
| veb1c.pk001.k11:fis | FIS | 92.4 | | | | | |
| epb3c.pk005.d14 | EST | | 60.7 | | | | |
| eae1s.pk003.b24:fis | FIS | | | | | | 176.0 |
| bdl1c.pk003.h16 | CGS | 154.0 | | | | | |
| p0037.crwbn23r:fis | GCS | 155.0 | | | | | |
| cbn10.pk0034.f8.f | CGS | | 160.0 | | | | |
| cpls1s.pk001.m19 | CGS | | 152.0 | | | | |

The data in Table 4 represents a calculation of the percent identity of the amino acid sequences set forth in SEQ ID NOs:2, 7, 9, 11, 13, 15, 17,19, 21,23, 25, 27, 29, 31, 33, 35, 37, 39, and 41, and the cytochrome P450 proteins from Arabidopsis [Arabidopsis thaliana] (NCBI General Identifier Nos. gi 7109461, [SEQ ID NO:42] which is identical to gi 12325138 and gi 15221132; and gi 11249511, [SEQ ID NO:44]; and gi 3831440, [SEQ ID NO:46]; and gi 8920576, [SEQ ID NO:47]), and a cytochrome P450 protein from orchid [Phalaenopsis sp.SM9108] (NCBI General Identifier No. gi 1173624, [SEQ ID NO:43]), and a cytochrome P450 protein from soybean [Glycine max] (NCBI General Identifier No. gi 5921926, [SEQ ID NO:45]).

TABLE 4
Percent Identity of Amino Acid Sequences
Deduced From the Nucleotide
Sequences of cDNA Clones Encoding Rice
Giant Embryo Cytochrome P450 and
Polypeptides Homologous To GE

| | Percent Identity to | | | | | |
|---|---|---|---|---|---|---|
| SEQ ID NO. | 7109461 | 1173624 | 11249511 | 5921926 | 3831440 | 8920576 |
| 2 | 49.1 | 59.6 | | | | |
| 7 | | 59.0 | | | | |
| 9 | | | 65.9 | | | |
| 11 | | 47.6 | | | | |
| 13 | | 67.0 | | | | |
| 15 | | 63.3 | | | | |
| 17 | | 62.0 | | | | |
| 19 | | | 53.2 | | 52.2% | |
| 21 | | | | 71.1 | | |
| 23 | | 67.1 | | | | |
| 25 | | | | 72.7 | | |
| 27 | | 53.4 | | | | |
| 29 | 68.1 | | | | | 68.8 |
| 31 | | | | | | 63.2 |
| 33 | | | 60.0 | | | |
| 35 | 62.7 | 68.8 | | | | |
| 37 | 73.6 | 75.0 | | | | |
| 39 | | 74.0 | | | | |
| 41 | | 67.1 | | | | |
| 93 | 49.6 | 61.3 | | | | |
| 95 | 47.5 | 61.7 | | | | |
| 97 | | 63.8 | | | | |
| 99 | | 61.3 | | | | |

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments and BLAST scores and probabilities indicate that the nucleic acid fragments comprising the instant cDNA clones encode a substantial portion of a plant cytochrome P450 protein that shares homology with the rice protein that gives rise to the giant embryo phenotype when mutated.

### Example 4

### Expression of Chimeric Constructs in Monocot Cells

A chimeric construct comprising a plant cDNA encoding the instant polypeptides in sense orientation with respect to promoter from the maize 27 kD zein, ubiquitin, or CaMV 35S, gene that is located 5′ to the cDNA fragment can be constructed. The 3′ fragment from the 10 kD zein gene [Kirihara et al. (1988) Gene 71:359-370] can be placed 3′ to the cDNA fragment. Such constructs are used to overexpress or cosuppress the gene(s) homologous to GE. It is realized that one skilled in the art could employ different promoters and/or 3′-end sequences to achieve comparable expression results. The construct with the CaMV 35S promoter is made as follows: the transcription termination element is released from the clone, In2-1 A, by BgIII and Asp718 digestion. The fragment is ligated to SphI and Asp718 restriction sites of pML141 [PCT Application No. WO 00/08162, published Feb. 17, 2000], which carries the 35S promoter, using the linker (GATCCATG) to connect BgIII and SphI ends. The DNA containing the GE ORF is amplified through PCR by using a primer set (5′-AGMTTCTTCCCATGGCGCTCTCCTCCAT-3′, SEQ ID NO:48; and 5′-AGAATTCTAGGCCCTAGCCACGGCCTTG-3′, SEQ ID NO:49) and the cDNA as a template. The fragment is then digested with EcoRI and inserted to the EcoRI site of the vector between the 35S promoter and the transcription terminator. The appropriate orientation of the insert is confirmed by sequencing.

The construct with the ubiquitin promoter is made as follows: the transcription termination element is released from the clone, In2-1 A, by Bcll and Kpnl digestion. The fragment is ligated to BamHI and NotI restriction sites of SK-ubi (BbsI), which carries the ubiquitin promoter (maize Ubi-1 promoter, Christensen and Quail (1996) Transgenic Res. 5: 213-218), using the linker (GGCCGTAC) to connect NotI and KpnI ends. The DNA containing the GE ORF is amplified through PCR by using a primer set (5′-AGGTCTCCCATGGCGCTCTCCTCCAT-3′, SEQ ID NO:50; and 5′-ATCATGATCTAGGCCCTAGCCACGGCCTTG-3′, SEQ ID NO:51) and the cDNA as a template. The fragment is then digested with BspHI and Bsal and inserted into the Bbsl site between the ubiquitin promoter and the transcription terminator.

Plasmid pML 103 has been deposited under the terms of the Budapest Treaty at ATCC (American Type Culture Collection, 10801 University Blvd., Manassas, Va. 20110-2209), and bears accession number ATCC 97366. The DNA segment from pML103 contains a 1.05 kb SaII-NcoI promoter fragment of the maize 27 kD zein gene [Prat et al. (1987) Gene 52:51-49; Gallardo et al. (1988) PlantSci. 54:211-2811] and a 0.96 kb SmaI-SaII fragment from the 3′ end of the maize 10 kD zein gene in the vector pGem9Zf(+) (Promega). Vector and insert DNA can be ligated at 15° C. overnight, essentially as described (Maniatis). The ligated DNA may then be used to transform E. coli XL1-Blue (Epicurian Coli XL-1 Blue™; Stratagene). Bacterial transformants can be screened by restriction enzyme digestion of plasmid DNA and limited nucleotide sequence analysis using the dideoxy chain termination method (Sequenase™ DNA Sequencing Kit; U.S. Biochemical). The resulting plasmid construct would comprise a chimeric construct encoding, in the 5′ to 3′ direction, the maize 27 kD zein promoter, a cDNA fragment encoding the instant polypeptides, and the 10 kD zein 3′ region.

The chimeric construct described above can then be introduced into corn cells by the following procedure. Immature corn embryos can be dissected from developing caryopses derived from crosses of the inbred corn lines H99 and LH132. The embryos are isolated 10 to 11 days after pollination when they are 1.0 to 1.5 mm long. The embryos are then placed with the axis-side facing down and in contact with agarose-solidified N6 medium (Chu et al. (1975) Sci. Sin. Peking 18:659-668). The embryos are kept in the dark at 27° C. Friable embryogenic callus consisting of undifferentiated masses of cells with somatic proembryoids and embryoids borne on suspensor structures proliferates from the scutellum of these immature embryos. The embryogenic callus isolated from the primary explant can be cultured on N6 medium and sub-cultured on this medium every 2 to 3 weeks.

The plasmid, p35S/Ac (obtained from Dr. Peter Eckes, Hoechst Ag, Frankfurt, Germany) may be used in transformation experiments in order to provide for a selectable marker. This plasmid contains the Pat gene (see European Patent Publication 0 242 236) which encodes phosphinothricin acetyl transferase (PAT). The enzyme PAT confers resistance to herbicidal glutamine synthetase inhibitors such as phosphinothricin. The pat gene in p35S/Ac is under the control of the 35S promoter from Cauliflower Mosaic Virus (Odell et al. (1985) Nature 313:810-812) and the 3′ region of the nopaline synthase gene from the T-DNA of the Ti plasmid of Agrobacterium tumefaciens.

The particle bombardment method (Klein et al. (1987) Nature 327:70-73) may be used to transfer genes to the callus culture cells. According to this method, gold particles (1 μm in diameter) are coated with DNA using the following technique. Ten μg of plasmid DNAs are added to 50 μL of a suspension of gold particles (60 mg per mL). Calcium chloride (50 μL of a 2.5 M solution) and spermidine free base (20 μL of a 1.0 M solution) are added to the particles. The suspension is vortexed during the addition of these solutions. After 10 minutes, the tubes are briefly centrifuged (5 sec at 15,000 rpm) and the supernatant removed. The particles are resuspended in 200 μL of absolute ethanol, centrifuged again and the supernatant removed. The ethanol rinse is performed again and the particles resuspended in a final volume of 30 μL of ethanol. An aliquot (5 μL) of the DNA-coated gold particles can be placed in the center of a Kapton™ flying disc (Bio-Rad Labs). The particles are then accelerated into the corn tissue with a Biolistic™ PDS-1000/He (Bio-Rad Instruments, Hercules Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covered a circular area of about 5 cm in diameter. The petri dish containing the tissue can be placed in the chamber of the PDS-1 000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardment the tissue can be transferred to N6 medium that contains bialophos (5 mg per liter) and lacks casein or proline. The tissue continues to grow slowly on this medium. After an additional 2 weeks the tissue can be transferred to fresh N6 medium containing bialophos. After 6 weeks, areas of about 1 cm in diameter of actively growing callus can be identified on some of the plates containing the bialophos-supplemented medium. These calli may continue to grow when sub-cultured on the selective medium.

Plants can be regenerated from the transgenic callus by first transferring clusters of tissue to N6 medium supplemented with 0.2 mg per liter of 2,4-D. After two weeks the tissue can be transferred to regeneration medium (Fromm et al. (1990) Bio/Technology 8:833-839).

### Example 5

### Expression of Chimeric Constructs in Dicot Cells

The 35S promoter of CaMV can be used to over-express and co-suppress the genes homologous to GE in dicot cells. For GE overexpression, the vector KS50 can be used to fuse the GE ORF to the 35S promoter. The GE ORF is amplified by PCR using the primer set with the Noti site at the 3′ end, AGCGGCCGCTTCCCATGGCGCTCTCCT, SEQ ID NO:52, and AGCGGCCGCTCAGGCCCTAGCCACGGC, SEQ ID NO:53. The amplified DNA fragment is digested with NotI and ligated into the NotI site of KS50. The correct orientation of the insert is determined by sequencing. KS50 (7,453 bp) is a derivative of pKS18HH (U.S. Pat. No. 5,846,784) which contains a T7 promoter/T7 terminator controlling the expression of a hygromycin phosphotransferase (HPT) gene, as well as a 35S promoter/NOS terminator controlling the expression of a second HPT gene. KS50 has an insert at the Sal I site consisting of a 35S promoter (960 bp)/NOS terminator (700 bp) cassette taken from pAW28, with a NotI cloning site between the promoter and terminator.

Soybean embryos may then be transformed with the expression vector comprising sequences encoding the instant polypeptides. To induce somatic embryos, cotyledons, 3-5 mm in length dissected from surface sterilized, immature seeds of the soybean cultivar A2872, can be cultured in the light or dark at 26° C. on an appropriate agar medium for 6-10 weeks. Somatic embryos which produce secondary embryos are then excised and placed into a suitable liquid medium. After repeated selection for clusters of somatic embryos which multiplied as early, globular staged embryos, the suspensions are maintained as described below.

Soybean embryogenic suspension cultures can be maintained in 35 mL liquid media on a rotary shaker, 150 rpm, at 26° C. with florescent lights on a 16:8 hour day/night schedule. Cultures are subcultured every two weeks by inoculating approximately 35 mg of tissue into 35 mL of liquid medium.

Soybean embryogenic suspension cultures may then be transformed by the method of particle gun bombardment (Klein et al. (1987) Nature (London) 327:70-73, U.S. Pat. No. 4,945,050). A DuPont Biolistic™ PDS1000/HE instrument (helium retrofit) can be used for these transformations.

A selectable marker gene which can be used to facilitate soybean transformation is a chimeric construct composed of the 35S promoter from Cauliflower Mosaic Virus (Odell et al. (1985) Nature 313:810-812), the hygromycin phosphotransferase gene from plasmid pJR225 (from E. coli; Gritz et al.(1983) Gene 25:179-188) and the 3′ region of the nopaline synthase gene from the T-DNA of the Ti plasmid of Agrobacterium tumefaciens. The seed expression cassette comprising the phaseolin 5′ region, the fragment encoding the instant polypeptides and the phaseolin 3′ region can be isolated as a restriction fragment. This fragment can then be inserted into a unique restriction site of the vector carrying the marker gene.

To 50 μL of a 60 mg/mL 1 μm gold particle suspension is added (in order): 5 μL DNA (1 μg/μL), 20 μL spermidine (0.1 M), and 50 μL CaCl₂ (2.5 M). The particle preparation is then agitated for three minutes, spun in a microfuge for 10 seconds and the supernatant removed. The DNA-coated particles are then washed once in 400 μL 70% ethanol and resuspended in 40 μL of anhydrous ethanol. The DNA/particle suspension can be sonicated three times for one second each. Five μL of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post bombardment with fresh media containing 50 mg/mL hygromycin. This selective media can be refreshed weekly. Seven to eight weeks post bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 6

### Fine Mapping of the ge Locus

The ge locus was mapped to the region around 85 cM on chromosome 7 using microsatellite and RFLP markers (Koh et al. (1996) Theor. Appl. Genet 93:257-261). Although numerous RFLP markers and YAC contigs have been mapped to rice chromosomes (Harushima et al. (1998) Genetics 148:479-494; http://rgp.dna.affrc.go.jp), the ge region was located in a 5 cM-long region where no physical markers were found so far. In order to map the ge locus, we made two mapping populations. The ge-3 (Japonica rice cv. Taichung 65) and ge-5 (Japonica rice cv. Kinmaze) homozygous mutant plants were chosen as female parents and Indica rice cultivar Kasalath as a male parent. The resulted F1 plants were selfed to obtain the F2 population. The ge F2 progeny (homozygous for ge) was selected from the F2 population.

To obtain F2 plants that carry recombinations near the ge locus, PCR-based DNA markers were developed. Several known RFLP markers were selected based on their map positions published by the Rice Genome Project Group (RGP) (Harushima et al. (1998) Genetics 148:479-494). The RFLP markers, R1245, R2677 and B2F2, were chosen for the distal markers and the markers, S1848 and C847, were chosen for the proximal markers. Primers were designed to amplify the genomic DNA corresponding to these markers, whose sequences were available from Genbank. For B2F2, which is a barley EST clone, rice homologues were obtained from the DuPont EST database as well as RGP EST database. The primers were designed based on the corresponding rice EST sequence.

A PCR reaction was carried out with 2 pmole primers of two dominant marker sets together, which were specific to the Kasalath sequence of C847 and B2F2. Young leaf tissues obtained from germinated ge F2 plants on N6 medium plates containing 0.3% gelrite were subjected to direct PCR reactions as described in Klimyuk et al. (1993) Plant J. 3:493-494 with modification of extending the sample boiling time to four minutes at the neutralization step. One 30 ul PCR reaction contained 2 ul 2.5 mM dNTPs, 2 ul 25 mM MgCl₂, 2 ul DNA extracted from leaf, 0.3 ul Amplitaq gold (Perkin Elmer) and 3 ul PCR buffer. The thermal cycle condition was 95° C. 10 min, 94° C. 30 sec, 56° C. 30 sec, 72° C. 30 sec, 72° C. 5 min repeating step 2 to 4 40 times. Amplification of Kasalath DNA was examined on 2.5 or 3% agarose gels.

By amplifying the marker regions from the parental Japonica and Indica cultivars, several single nucleotide polymorphisms (SNPS) were found. To develop a dominant PCR-based DNA marker from the distal side, one SNP found in C847 was chosen. At this SNP the Japonica sequence had an A residue, whereas the Indica sequence had T. The primer (5′GTTTCATMTGAAATTGACTCTTTTTCAGTM3′; SEQ ID NO:54) was designed in a way that the Indica-specific base was complementary to its 3′ end. Using this and the other primer (5′GCAAATMTTATTTCTATATACAGGACAGGC3′; SEQ ID NO:55) as a set, the corresponding DNA could be amplified only from the Indica. For the proximal side, the B2F2 rice homologue was chosen, which carried a SNP between Japonica (A) and Indica cultivars (T). The designed primer (5′TAGCTTTAGAGTACATTTCTTAGATACGGCA3′; SEQ ID NO:56) was complementary to the Indica sequence at its 3′ end. In combination with another primer (5′TTACTTTGAGCGTGCCMGCAGTATAATTTCT3′; SEQ ID NO:57), DNA was amplified only from Indica but not from Japonica.

By using these Indica-specific primer pairs, 1290 ge homozygous F2 were screened, and 33 recombinants in total were obtained, 15 from the proximal and 18 from the distal ge region.

### Example 7

### Map-Based Cloning of GE

To obtain the closest physical marker which could serve as a starting point of the chromosome walk toward GE, DNA was isolated from the ends of three YAC clones, Y1931, Y4052 and Y4566. These clones were previously mapped to the region relatively close to the ge locus by RGP. Using a PCR-based method, we recovered and sequenced the both ends of Y4052 and Y1931 and left end of Y4566 (see Methods and Materials). By using primer sets specific to each isolated end, the orientation and overlaps of these YAC clones were analyzed and it was established that the Y4052 left end is the far-most end of the contig of Y4052 and Y4566. To determine which end of Y4052 is close to the ge locus, RFLP was developed for each end. The segregation analysis of ten recombinants from the distal region showed that the Y4052 left end was closer to ge than the right end, leaving 3 and 9 recombination breakpoints, respectively.

Total DNA from yeast YAC strains was extracted. 100 ng DNA was digested by Alul, HaeIII and RsaI, and ligated with the vectorette adaptor (5′MGGAGAGGACGCTGTCTGTCGMGGTMGGAACGGACGAGAGAAGGG3′; SEQ ID NO:58; and 5′CTCTCCCTTCTCGAATCGTAACCGTTCGTACGAGAATCGCTGTCCTCTCCTT3′; SEQ ID NO:59). 10 ng of ligated DNA was used as PCR template to amplify YAC ends. One PCR reaction contained 20 pmole of the primer specific to the left YAC arm (5′CACCCGTTCTCGGAGCACTGTCCGACCGC3′; SEQ ID NO:60; or the primer specific to the right arm (5′ATATAGGCGCCAGCAACCGCACCTGTGGCG3′; SEQ ID NO:61) with 1.6 mM MgCl₂, 50 mM KCl, 10 mM Tris-HCl (pH 9.0), 0.01% gelatin and 2.5 mM dNTPs. The cycle condition was 95° C. 10 min, 92° C. 1 min, 60° C. 1 min, 72° C. 1 min. After completing 10 cycles of step 2 through 4, the vectorette specific primer was (5′CGMTCGTAACCGTTCGTACGAGAATCGCT3′; SEQ ID NO:62) was added to the reaction and further amplified in the condition of 92° C. 1 min, 60° C. 1 min and 72° C. 3 min for 30 cycles. The PCR products were separated on agarose gels and amplified DNA was extracted for the second PCR amplification. The second PCR was carried out with the presence of 16 pmole the primer specific to the vectorette unit and 30 pmole the nested primer specific to the YAC left end (5′CTGMCCATCTTGGAAGGAC3′; SEQ ID NO:63) or the primer specific to the right end (5′ACTTGCMGTCTGGGMGTG3′; SEQ ID NO:64). The cycling condition was 95° C. 10 min, 94° C. 1 min, 58° C. 1 min, 72° C. 1 min, repeating step 2 to step 4 20 times. The recovered ends were cloned into pGEM-T Easy (Promega) and sequenced. The primers derived from the end sequences were used for analyzing the overlapped structure of the YAC contig. Also, these DNA fragments were used to find RFLP to map them with respect to the ge locus.

Based on these results, we initiated a chromosome walk from the Y4052 left end. Two Texas A&M BAC libraries made from the genomic DNA of Taquiq (TQ Indica rice) and Lemont (LM Japonica rice) were used to screen corresponding clones by DNA blot hybridization. Two BAC clones were recovered, TQ1-19L and TQ22-7E, using the Y4052 left end as a probe. The ends of BAC clones were recovered by TAIL PCR and the recovered DNA fragments were cloned into pGEM-T Easy for sequencing (see Materials Methods). Using these sequences, BAC end-specific primer sets were designed and the orientation of these BAC clones in the contig was determined. The data of the PCR analysis showed that the right end (the SP6 side) of TQ1-19L was the new closest end to ge, not present in TQ22-7E and the YAC clones.

The right end of TQ1-19L was used for the second screening of overlapping BAC clones. Three BACs were obtained, LM10-22N, LM10-11O and LM15-7P. The process of recovering BAC ends and mapping per PCR was repeated. For the third screen, the left end was used (the T7 side) of LM15-7P and LM3-6B was obtained. Forthe fourth screen, the left end of LM3-6B was used and LM20-4D, LM17-3H were obtained. The left end of LM20-4D was mapped to the end of the contig. For the fifth screen, this end was not used as a probe to obtain overlapping BAC clones because of the presence of a repetitive sequence. To obtain an appropriate DNA probe from LM204D, the BAC clone was digested by restriction enzyme HindIll and subcloned into pUC18. By DNA blot analysis, one 1.6 kb-long fragment was found not present on the other overlapping clone, LM3-6B, indicating that the fragment was localized toward the end the BAC contig. The 1.6 kb HindIII fragment was used as a probe for the fifth screen and TQ18-1I and LM2-15J were isolated as the overlapping clones. In the sixth screening, the left end of TQ18-1I was used as a probe and two BAC clones, LM4-12E and LMI5-20J, were isolated.

The blots of two Texas A&M BAC libraries made from Taquiq, Indica rice; and Lemont, Japonica rice were hybridized with DNA probes using standard DNA hybridization conditions (Sambrook et al. (1989) “Molecular Cloning” Cold Spring Harbor Laboratory Press, New York). The ends of BAC clones, which were made using the pBeloBAC11 vector, were recovered by TAIL PCR. A typical TAIL PCR reaction was carried out in 20 ul, containing a BAC vector specific primer (4 pmole) and arbitrary degenerated (AD) primers (50 pmole) with 0.2 ul expand hi fidelity Taq polymerase (Roche). Six nested primers specific to the BAC vector were designed:

| | | |
|---|---|---|
| BACL1; | ATTCAGGCTGCGCAACTGTTG | SEQ ID NO:65 |
| BACL2; | CTGCAAGGCGATTAAGTTGG | SEQ ID NO:66 |
| BACL3; | GGGTTTTCCCAGTCACGAC | SEQ ID NO:67 |
| BACR1; | TGAGTTAGCTCACTCATTAGGGAC | SEQ ID NO:68 |
| BACR2; | GCTTCCGGCTCGTATGTTGTG | SEQ ID NO:69 |
| BACR3; | GACCATGATTACGCCAAGC | SEQ ID NO:70 |

Seven different AD primers (AD1-7) were used as designed by Liu and Whittier (1995) Genomics 25:674-681, and Liu et al. (1995) Plant J. 8:457-463:

| | | |
|---|---|---|
| AD1; | TGWGNAGWANCASAGA | SEQ ID NO:71 |
| AD2; | AGWGNAGWANCAWAGG | SEQ ID NO:72 |
| AD3; | CAWCGICNGAIASGAA | SEQ ID NO:73 |
| AD4; | TCSTICGNACITWGGA | SEQ ID NO:74 |
| AD5; | NGTCGASWGANAWGAA | SEQ ID NO:75 |
| AD6; | GTNCGASWCANAWGTT | SEQ ID NO:76 |
| AD7; | WGTGNAGWANCANAGA | SEQ ID NO:77 |

The condition of the first-round PCR was as described by Liu and Whittier 1995, and Liu et al. 1995 with modification of the annealing temperatures changing to 65° C. for the first 5 cycles and 61° C. for the last 15 cycles. In the second PCR, we used 1 ul 1/30 diluted 1^{st} PCR product as a template. The 20 ul reaction contained 8 pmole 2^{nd} BAC vector specific primer, 25 pmole AD primer, and 0.2 ul expand hi fidelity Taq polymerase. The condition of thermal cycle was as described by Liu and Whittier 1995, and Liu et al. 1995 with modification of the annealing temperatures changing to 60° C. for the first two cycles.

3^{rd} PCR was carried out with a normal PCR thermal cycle steps. The reaction contained the 3^{rd} BAC vector specific primer and AD primers. PCR product was cloned into pGEM-T easy vector (Promega) and their DNA sequence was determined by conventional sequencing methods.

Several DNA fragments isolated from these BAC clones that showed polymorphisms between the Japonica and Indica cultivars were used to map recombination break points of the isolated recombinants. As a result, the 1.6 kb HindIII fragment LM20-4D gave three recombination break points, whereas a 950 bp HindIII fragment of TQ18-1I gave no break point among the fifteen distal recombinants. Since the same fragment of TQ18-1I gave one break point among the proximal recombinants, the ge locus was mapped between two makers, 1.6 kb HindlIl of LM20-4D and 950 bp HindIII of TQ18-1I, i.e. on the two BAC clones, LM20-4D and TQ18-11.

### Example 8

### Identification of the GE Gene

In order to identify the GE gene that was mapped to the region comprising two BAC clones, LM20-4D and TQ18-1I, the whole genomic insert of these BAC clones was sequenced. For the purpose, BAC DNA was nebulized using high-pressure nitrogen gas as described in Roe et al. 1996 (Roe et al. (1996) “DNA isolation and Sequencing” John Wiley and Sons, New York). DNA fragments with the length of 1-2 kb were recovered from agarose gels and cloned into pUC18. 686 clones derived from LM20-4D were randomly isolated and sequenced. Likewise, 700 clones derived from TQ1I-18 were isolated and sequenced. Twelve groups of contiguous sequences were obtained from LM20-4D and 16 from TQ1I-18. Most gaps were filled by PCR and also by obtaining other subclones derived from HindIII or EcoRI fragments of LM20 4D and LM4-12E. This resulted in the construction of a 90 kb-long continuous sequence between two DNA markers, 1.6 kb HindIII LM20-4D and 950 bp HindIII TQ18-1I.

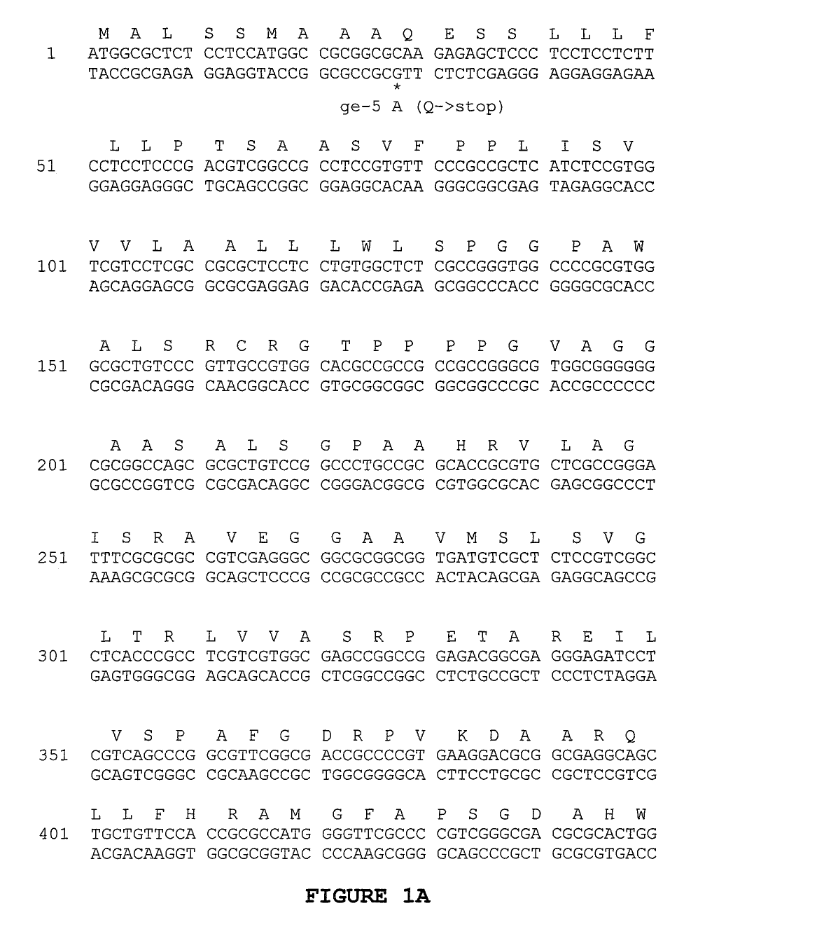

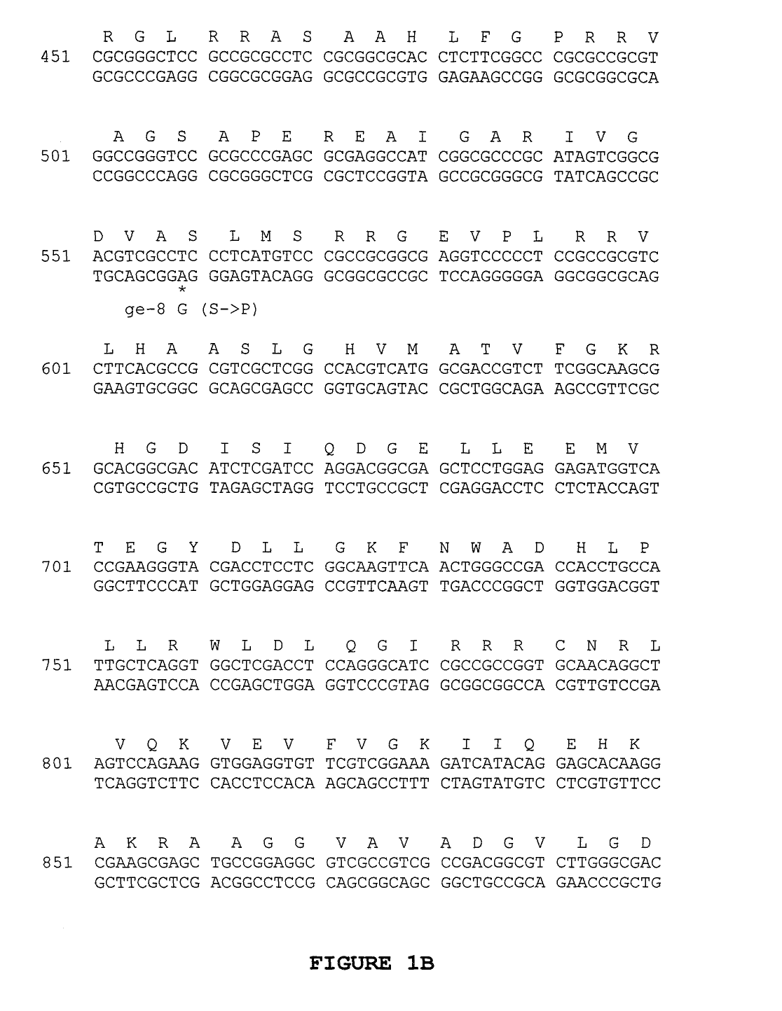

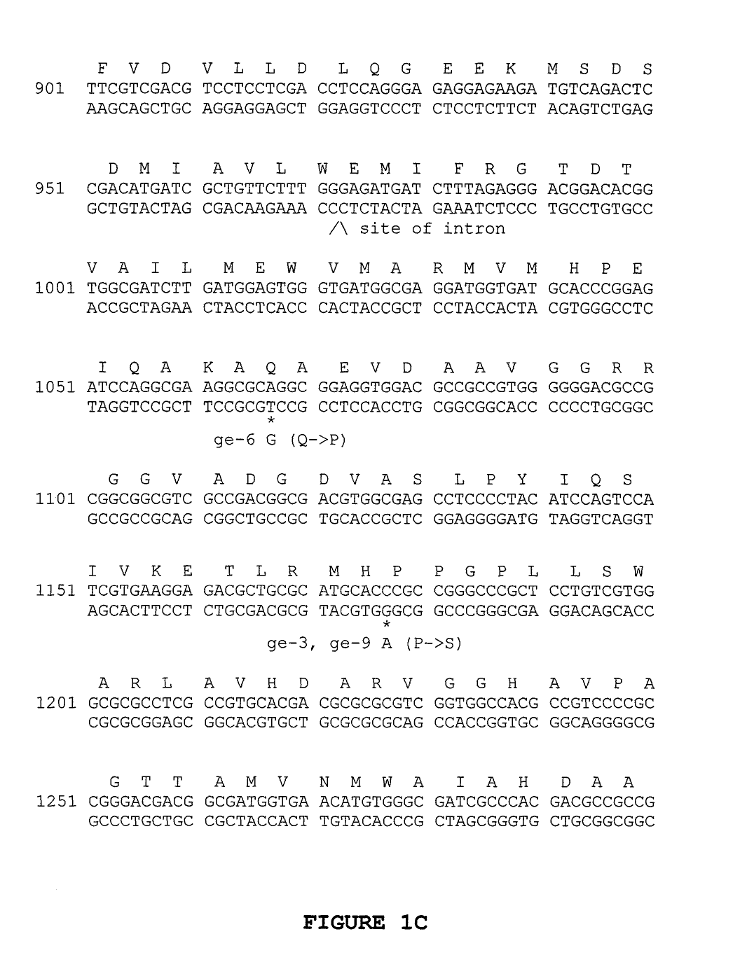

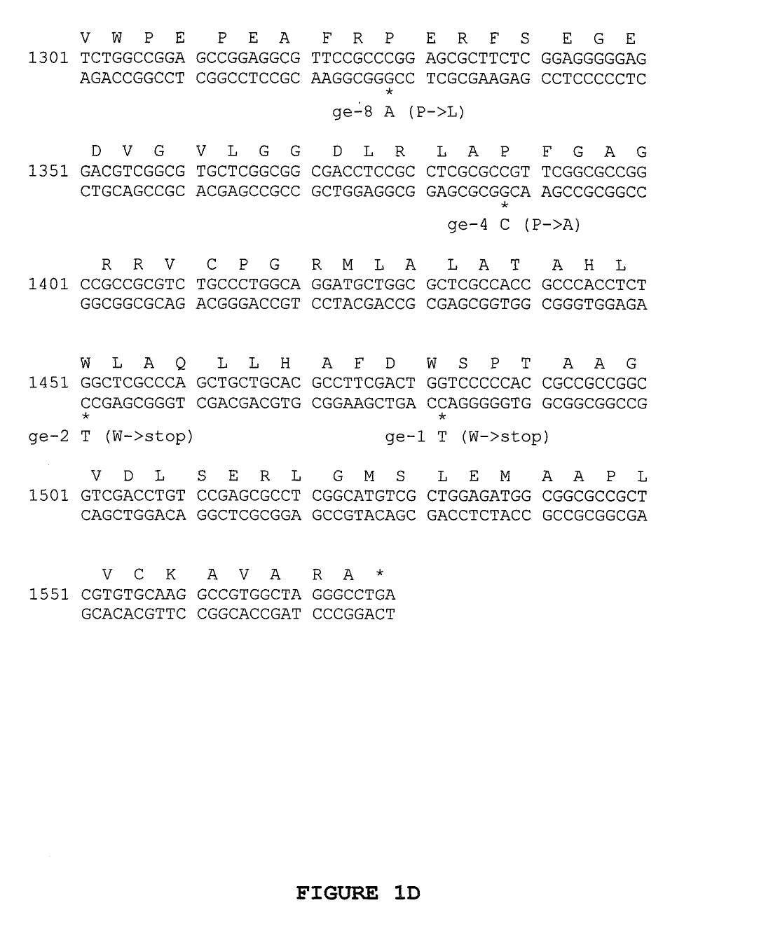

Within the 90 kb sequence, more than ten regions showing certain similarities to genes filed in Genbank as well as in our EST database were identified. Judging from the number of recombinants at the end of the region and the location of these ORFs, one ORF encoding a protein similar to CYP78 proteins, a subfamily of P450 proteins, was found to be a candidate for the GE gene. To confirm the correlation between GE and the P450 gene, the genomic region from mutants and wild type were amplified by PCR. Comparing these sequences, mutations of nine different alleles were identified, all of which were found in the ORF of the P450 gene; three nonsense and six mis-sense mutations were found (see FIG. 1). These data confirm that this rice cytochrome P450 gene is the GE gene, and that mutations within this gene can result in a GE phenotype.

There are a number of P450 genes from GenBank shown to be homologous to GE. Some of them are also expressed in ovules or shoot meristems (Nadeau et al. (1996) Plant Cell 8:213-239; Zondlo and Irish (1999) Plant J. 19:259-268). However, the function of these genes remains largely unknown. In one case, an Arabidopsis gene homologous to GE was overexpressed and the resulting fruit, or pericarp, became enlarged while forming few, if any, seeds or embryos (Ito and Meyerowitz (2000) Plant Cell 12:1541-1550). However, the disruption of this Arabidopsis gene caused no phenotype. It is believed that the characterization, in the present invention, of the rice cytochrome P450 gene as “giant embryo” represents the first example of a plant gene directly controlling embryo size.

### Example 9

### Cloning the cDNA Encoding Cytochrome P450 Protein Associated with the Giant Embryo Phenotype

Total RNA was extracted from developing rice seeds harvested 2-5 days after pollination, using a TRIazol® Reagent obtained from Life Technologies Inc., Rockville, Md., 20849 (GIBCO-BRL) which contains phenol and guanidine thiocyanate. Poly A mRNA was purified from total RNA with mRNA Purification kits obtained from Amersham Pharmacia Biotech Inc., Piscataway, N.J., 08855, which consists of oligo (dT)-cellulose spin columns. To make the cDNA library, 5.5 ug of polyA RNA was used for cDNA synthesis kits obtained from Stratagene, La Jolla, Calif., 92037. Superscript® reverse transcriptase obtained from Life Technologies Inc., Rockville, Md., 20849 (GIBCO-BRL) was substituted for the MMLV reverse transcriptase in the first step. BRL cDNA Size Fraction Columns (GIBCO-BRL) were used to fractionate the cDNA by size, fraction 1 to 13 were precipitated, resuspended and ligated with 1 ug of the Uni-ZAP XR vector. After two days of ligation it was packaged in Gigapack III Gold® packaging extract obtained from Stratagene, La Jolla, Calif., 92037. The unamplified library titer was approximately 780,000 plaques per ml. The entire amount was used for amplification purposes and the procedure produced 150 mis of an amplified cDNA library with a titer of 5.5×10⁸ pfu/ml.

Screening for the GE cDNA followed standard protocols well known to those skilled in the art (Ausubel et al. 1993, “Current Protocols in Molecular Biology” John Wiley & Sons, USA, or Sambrook et al. 1989. Molecular Cloning: A Laboratory Manual. Cold Spring Harbor Laboratory Press). Briefly, 1.5×10⁶ phage clones were plated, then transferred to nylon membranes, which were then subjected to hybridization with radioactively labeled GE probe. More than five positives were detected per 50,000 plaques. Approximately 125 positives were isolated and examined for their identity as GE cDNAs through PCR with GE-specific primers. One primer specific to the 5′ end of the isolated nucleic acid fragment (GGGMGCGTTCGCGAAGTGAG, SEQ ID NO:78) and the other specific to the cloning vector next to the 5′ end of the cDNA insert (AGCGGATAACMTTTCACACAGG, SEQ ID NO:79). Six of the longest cDNA clones that gave positive results from the PCR reaction were isolated and sequenced. All six clones have nearly the same length, the longest cDNA being 28 nucleotides upstream of the ATG start codon predicted from the genomic sequence.

### Example 10

### Genetic Confirmation of the GE Gene

The genetic confirmation that the rice cytochrome P450 isolated nucleic acid fragment encoded the polypeptide responsible for the giant embryo phenotype was accomplished by transforming ge mutants with the isolated cytochrome P450 cloned sequence. This experiment confirmed that the cytochrome P450 is the GE gene, and that the genomic region used in the transformation contained the complete set of regulatory elements necessary for normal GE expression. The genomic DNA used for the transformation covered 1.7 kb upstream of the coding region, the coding region of GE, and 1.6 kb downstream of the coding region.

GE homologs from other crop species can also be tested in this system by obtaining full-gene sequences, and complementing the rice GE mutant.

In order to confirm possible tissue-specific expression of the GE gene, the presence of the GE transcript in various tissues was analyzed by RNA blot analysis and in situ hybridization (see Example 11).

One method for transforming DNA into cells of higher plants that is available to those skilled in the art is high-velocity ballistic bombardment using metal particles coated with the nucleic acid constructs of interest (see Klein et al. Nature (1987) (London) 327:70-73, and see U.S. Pat. No. 4,945,050). A Biolistic PDS-1000/He (BioRAD Laboratories, Hercules, Calif.) was used for these complementation experiments (see Example 4 for further details). The particle bombardment technique was used to transform the ge mutant with a 5.1 kb EcoRI fragment from wild type (nucleotides 6604-11735 of SEQ ID NO:3) that includes 1.7 kb upstream of the GE coding region, the GE coding region plus intron, and 1.6 kb downstream of the GE coding region.

The bacterial hygromycin B phosphotransferase (Hpt II) gene from Streptomyces hygroscopicus that confers resistance to the antibiotic hygromycin was used as the selectable marker for the rice transformation. In the vector, pML18, the Hpt II gene was engineered with the 35S promoter from Cauliflower Mosaic Virus and the termination and polyadenylation signals from the octopine synthase gene of Agrobacterium tumefaciens. pML18 was described in WO 97/47731, which was published on Dec. 18, 1997, the disclosure of which is hereby incorporated by reference.

Embryogenic callus cultures derived from the scutellum of germinating rice seeds serve as source material for transformation experiments. This material was generated by germinating sterile rice seeds on a callus initiation media (MS salts, Nitsch and Nitsch vitamins, 1.0 mg/l 2,4-D and 10 μM AgNO₃) in the dark at 27-28° C. Embryogenic callus proliferating from the scutellum of the embryos was then transferred to CM media (N6 salts, Nitsch and Nitsch vitamins, 1 mg/l 2,4-D, Chu et al., 1985, Sci. Sinica 18: 659-668). Callus cultures were maintained on CM by routine sub-culture at two week intervals and used for transformation within 10 weeks of initiation.

Callus was prepared for transformation by subculturing 0.5-1.0 mm pieces approximately 1 mm apart, arranged in a circular area of about 4 cm in diameter, in the center of a circle of Whatman #541 paper placed on CM media. The plates with callus were incubated in the dark at 27-28° C. for 3-5 days. Prior to bombardment, the filters with callus were transferred to CM supplemented with 0.25 M mannitol and 0.25 M sorbitol for 3 hr in the dark. The petri dish lids were then left ajar for 20-45 minutes in a sterile hood to allow moisture on tissue to dissipate.

Each genomic DNA fragment was co-precipitated with pML18 containing the selectable marker for rice transformation onto the surface of gold particles. To accomplish this, a total of 10 μg of DNA at a 2:1 ratio of trait:selectable marker DNAs were added to 50 μl aliquot of gold particles that were resuspended at a concentration of 60 mg ml⁻¹. Calcium chloride (50 μl of a 2.5 M solution) and spermidine (20 μl of a 0.1 M solution) were then added to the gold-DNA suspension as the tube was vortexed for 3 min. The gold particles were centrifuged in a microfuge for 1 sec and the supernatant removed. The gold particles were then washed twice with 1 ml of absolute ethanol and then resuspended in 50 μl of absolute ethanol and sonicated (bath sonicator) for one second to disperse the gold particles. The gold suspension was incubated at −70° C. for five minutes and sonicated (bath sonicator) if needed to disperse the particles. Six μl of the DNA-coated gold particles were then loaded onto mylar macrocarrier disks and the ethanol was allowed to evaporate.

At the end of the drying period, a petri dish containing the tissue was placed in the chamber of the PDS-1 000/He. The air in the chamber was then evacuated to a vacuum of 28-29 inches Hg. The macrocarrier was accelerated with a helium shock wave using a rupture membrane that bursts when the He pressure in the shock tube reaches 1080-1100 psi. The tissue was placed approximately 8 cm from the stopping screen and the callus was bombarded two times. Two to four plates of tissue were bombarded in this way with the DNA-coated gold particles. Following bombardment, the callus tissue was transferred to CM media without supplemental sorbitol or mannitol.

Within 3-5 days after bombardment the callus tissue was transferred to SM media (CM medium containing 50 mg/l hygromycin). To accomplish this, callus tissue was transferred from plates to sterile 50 ml conical tubes and weighed. Molten top-agar at 40° C. was added using 2.5 ml of top agar/100 mg of callus. Callus clumps were broken into fragments of less than 2 mm diameter by repeated dispensing through a 10 ml pipet. Three ml aliquots of the callus suspension were plated onto fresh SM media and the plates were incubated in the dark for 4 weeks at 27-28° C. After 4 weeks, transgenic callus events were identified, transferred to fresh SM plates and grown for an additional 2 weeks in the dark at 27-28° C.

Growing callus was transferred to RM1 media (MS salts, Nitsch and Nitsch vitamins, 2% sucrose, 3% sorbitol, 0.4% gelrite +50 ppm hyg B) for 2 weeks in the dark at 25° C. After 2 weeks the callus was transferred to RM2 media (MS salts, Nitsch and Nitsch vitamins, 3% sucrose, 0.4% gelrite +50 ppm hyg B) and placed under cool white light (˜40 μm⁻²s⁻¹) with a 12 hr photoperiod at 25° C. and 30-40% humidity. After 2-4 weeks in the light, callus began to organize, and form shoots. Shoots were removed from surrounding callus/media and gently transferred to RM3 media (1/2×MS salts, Nitsch and Nitsch vitamins, 1% sucrose +50 ppm hygromycin B) in phytatrays (Sigma Chemical Co., St. Louis, Mo.) and incubation was continued using the same conditions as described in the previous step.

Plants were transferred from RM3 to 4″ pots containing Metro mix 350 after 2-3 weeks, when sufficient root and shoot growth had occurred. The seed obtained from the transgenic plants was examined for genetic complementation of the ge mutation with the wild-type genomic DNA containing the GE gene. The mutant GE line transformed with the 5.1 kb EcoRI fragment containing the wild-type GE isolated nucleic acid fragment yielded rice grains with normal embryos.

This result confirms that the 5.1 kb EcoRI fragment containing the cytochrome P450 coding region is sufficient to complement the ge mutant phenotype. Furthermore, all regulatory elements necessary for “wild-type” expression of the gene are apparently present within the 5.1 kb EcoRI fragment, since this region completely complements the ge mutation.

### Example 11

### Characterization of the GE promoter

The 5.1 kb EcoRI genomic fragment described in Example 10 was sufficient to complement the ge mutation. This demonstrated that the promoter, required for the proper GE expression, was encoded in this genomic region. Two corn homologs of the rice GE are described in Example 13. The 2 kb upstream sequences from both of these genes, zmGE1 and zmGE2, are shown in SEQ ID NOs:104 and 105, respectively. It is believed that the regulatory elements necessary for normal maize GE expression are contained within SEQ ID NO:104 or 105 and the coding regions for zmGE1 and zmGE2.

In order to investigate the expression pattern necessary for GE function, the accumulation of GE RNA in tissues was analyzed by means of in situ hybridization. To obtain detailed data of weak GE expression, a radioactive method following the protocol of Sakai et al. (1995) Nature 378:199-203) was employed. Plant materials were fix and embedded in paraplast according to Jackson, D. P. (1991) In Situ Hybridization in Plants. In: “Molecular Plant Pathology: A Practical Approach”, (Bowles, D. J., Gurr, S. J. and McPhereson, M. eds), Oxford University Press. The sections were prepared in 8-μm thickness using a rotary microtome. To detect GE-specific sense RNA, the region containing the 3′UTR was amplified by PCR and cloned into pGEM-T (Promega). The primers used to amplify the region for the probe were GE3′RVQ: TCGTGTGCMGGCCGTGGCTA (SEQ ID NO:106) and GE3′LVC: GCACGATCCATTTAGCACACCAG (SEQ ID NO:107). The amplified sequence was from nucleotide 9941 to 10300 of SEQ ID NO:3.

The antisense RNA probe to detect sense GE RNA was synthesized by linearizing the clone by digesting with SpeI and transcribing with T7 RNA polymerase. The sense RNA for control was synthesized by linearizing the clone by digesting with NcoI and transcribing with SP6 RNA polymerase.

After three weeks of exposure on NBT2 Kodak autoradiography emulsion film, the result was analyzed through dark field microscopy using a compound microscope (Nikon, Eclipse E800). GE RNA accumulation was detected in the developing embryo as well as endosperm tissues. The earliest expression detected was at two day after pollination. GE expression detected in embryos was restricted to the apical region at the globular stage and to the epidermal layer of scutellum facing to the endosperm tissue at coleopilar and late stages. In the developing endosperm before the cellular stage, GE RNA was detected in the entire region with some concentration in the area close to the embryonic tissue. Later, the GE expression pattern shifted, with more expression seen in the area facing the embryo. Furthermore, GE expression was also detected in very young leaf tissues.

### Example 12

### Identification of the Barley GE Homolog

In order to identify the gene, a barley genomic library (Stratagene, Catalogue No. 946104) was screened by hybridizing a DNA probe made from the entire GE isolated nucleic acid fragment at 65° C. and washing at a medium stringency (5× SSPE, 0.5% SDS at 65° C. followed by 1× SSPE, 0.5× SDS, 65° C.). Five positively hybridizing lambda clones were isolated. Mapping of these clones via restriction enzyme digestion confirmed that all five were overlapping clones from the same genomic region. The DNA fragment that contained the region homologous to rice GE was further subcloned and sequenced.

The deduced coding sequence and the deduced translation product of the barley GE homolog are shown in SEQ ID NO:92 and 93, respectively. The barley GE homolog has a high degree of conservation to the rice GE protein (72.9% identity based on the Clustal method of alignment). Furthermore, the 91 nucleotide intron found in the rice GE gene is conserved in its placement within the barley gene (between nucleotides 991 and 992 of SEQ ID NO:92, the barley intron is 125 nucleotides). This conservation of intron placement is also found in zmGE1, zmGE2, and zmGE3 (see Example 13).

### Example 13

### Identification of Maize GE Homologs

Maize GE homologs were identified by analysis of EST clones with strong homologies to GE (see EXAMPLE 3). Two genes represented by ESTs, cbn10.pk0034.f8, maize GE2 (zmGE2, SEQ ID NO:96 for the nucleotide coding sequence, and SEQ ID NO:97 for the putative translation product) and p0121.cformn62r, maize GE1 (zmGE1, SEQ ID NO:94 for the nucleotide coding sequence, and SEQ ID NO:95 for the putative translation product), were shown to be the most homologous genes in the maize genome by the cross-hybridization analysis. A third clone cpls1s.pk001.m19 (zmGE3, SEQ ID NO:98 for the nucleotide coding sequence, and SEQ ID NO:99 for the putative translation product) has also been identified by analyzing BAC genomic clones (see below). There is a single intron contained within each of the three maize genes, and its placement is conserved with respect to the rice and barley genes discussed in Example 12. The intron for zmGE1 is 122 nucleotides and is found between nucleotides 1143 and 1144 of SEQ ID NO:94, the intron for zmGE2 is 193 nucleotides and is found between nucleotides 942 and 943 of SEQ ID NO:96, and the size of the intron for zmGE3 has not yet been determined, although it is considerably larger than the other four.

For the cross-hybridization analysis, as described below, maize DNA was digested with several different restriction enzymes and separated on 0.7% agarose gel. DNA was transferred to a nylon membrane filter, HyBond N (Amersham), and hybridized at 50° C. with the ³²P-labeled probe made from the whole coding region of the rice GE gene. After washing the filter at 1× SSPE, 0.5% SDS at 65° C., it was exposed on the Phospho Imager screen (Molecular Dynamics) and signals were detected by using Phospho Imager scanner (Molecular Dynamics). The signals were detected from more than one band, indicating the possibility that there was more than one maize genes very homologous to rice GE.

To identify the homologous genes in the maize genome, the maize genomic library (Stratagene, Catalog No. 946102) was screened at the medium stringency condition starting at 2× SSPE, 0.5% SDS, 50° C. and then at 1× SSPE, 0.5% SDS 65° C., and obtained nine lambda clones that gave distinct positive signals. PCR analysis showed these clones were shown to have sequences specific to either cbn10.pk0034.f8 or p0121.cformn62r, proving that these EST clones encoded the corn genes most homologous to rice GE.

In order to obtain further information on the structure of these genes represented by two EST clones, maize genomic BAC clones were screened. The clone, p0121.cformn62r, hybridized to BAC clones that belonged to one contig. The clone, cbn10.pk0034.f8, hybridized to BAC clones that derived from two distinct contigs. One BAC clone from each contig was chosen and subclones for sequencing were made of whole BAC inserts. These BACs were BAC b94d.b2 for p0121.cformn62r (zmGE1) and BACs b153c.j17 and b37c.f1 for cbn10.pk0034.f8 contigs (zmGE2). The sequence of each BAC revealed the genomic structure of maize GE homologs. The BAC b37c.f1 contained ORF nearly identical but distinct sequence to the gene represented by cbn10.pk0034.f8 and BAC b153c.j17. The third corn homolog was named zmGE3.

### Example 14

### Identification of a GE Homolog by Genomic Synteny Analysis

Synteny analysis, or the conservation of gene placement on chromosomes between different organisms, is known to be a useful tool for identifying homologous genes or genomic regions from one species by comparison to a known genomic region from another closely related species. For instance, GeneA from corn is known to possess a unique activity but is related to a large multigene family. Chromosomal analysis of GeneA shows that it is closely linked to GeneB. If one wanted to find the homolog of GeneA in rice (GeneA-r), it is likely that the member of the GeneA-r family will be closely linked to GeneB-r. Rice and maize are known to exhibit conservation of chromosomal structures, i.e. gene orders, to a large extent (Ahn and Tanksley PNAS (1993) 90:7980-7984). In order to make use of such synteny relationships to identify homologs among closely related species, the genomic sequence of the three BACs described in EXAMPLE 13 were compared to the 100 kb-long, rice GE genomic sequence described in EXAMPLE 1. The analysis revealed ORFs in BAC b94d.b2, showing a similarity to a hydrolase, a gene closely linked to the rice GE (the rice hydrolase gene is shown in SEQ ID NO:100 and 101, nucleotide and polypeptide, respectively; and the maize hydrolase is shown in SEQ ID NO:102 and 103). Therefore, zmGE1 is closely linked to a hydrolase gene, just like the rice GE gene. This demonstrated that rice genes closely linked to GE could be used as tags to isolate GE homologs from plant species that have conserved chromosomal structures by using synteny.

### Example 15

### Identification of Protein Sequences Specific to GE and GE Homologs

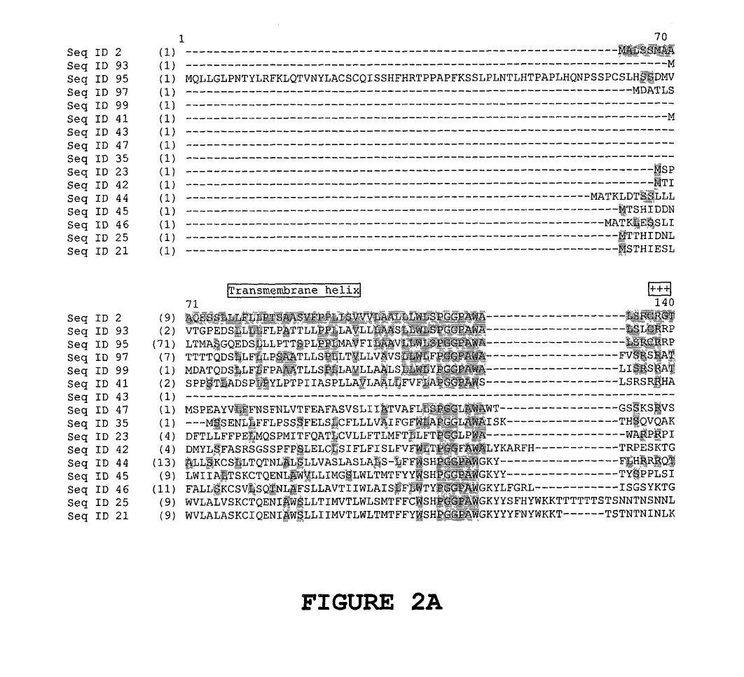

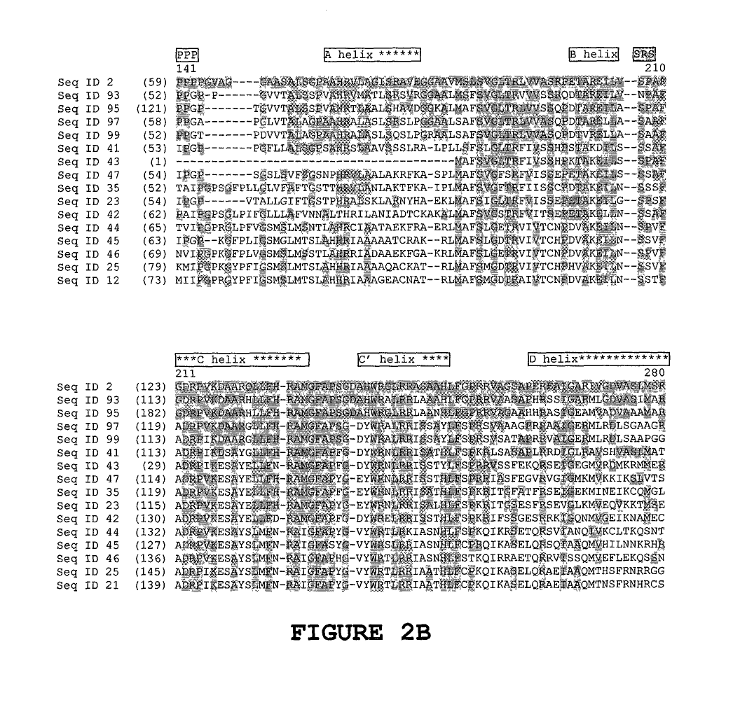

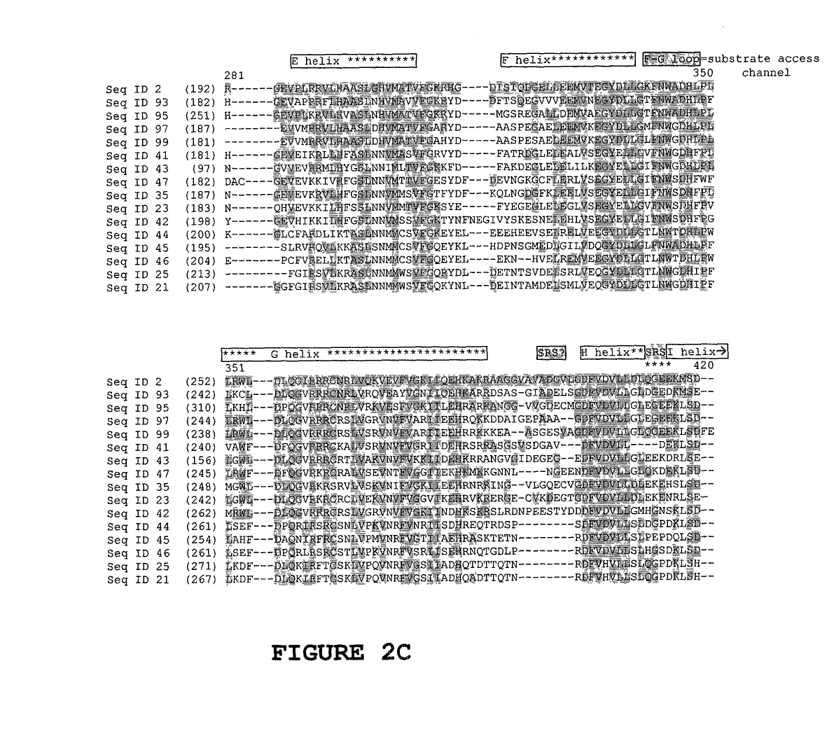

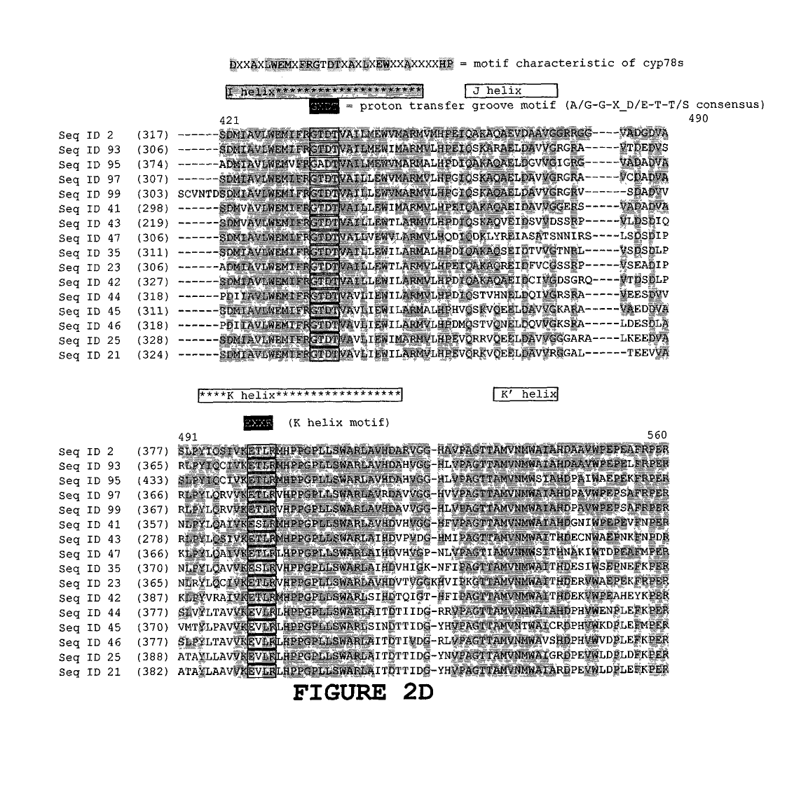

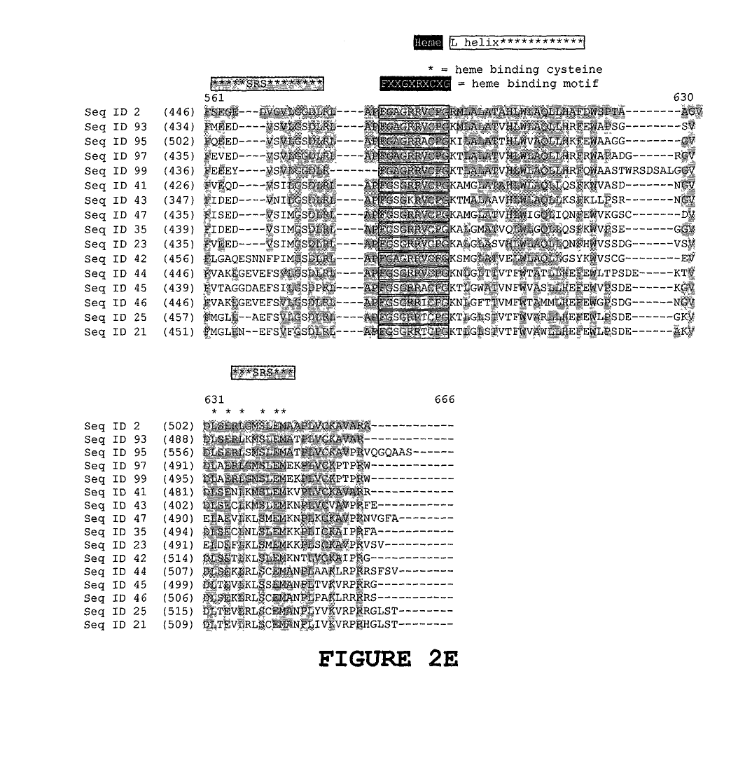

Cytochrome P450 proteins comprise a superfamily of genes with a variety of functions (Werck-Reichhart and Feyereisen (2000) Genome Biology 1:reviews 3003.1-3003.9). FIG. 2 shows an alignment of the rice GE (SEQ ID NO:2), barley GE-homolog (SEQ ID NO:93), maize GE1-homolog (SEQ ID NO:95), maize GE2-homolog (SEQ ID NO:97), maize GE3-homolog (SEQ ID NO:99), lily GE-homolog (SEQ ID NO:41), orchid gi 1173624 (SEQ ID NO:43), Arabidopsis gi 1235138 (SEQ ID NO:42), Arabidopsis gi 8920576 (SEQ ID NO:47), columbine GE-homolog (SEQ ID NO:35), soybean GE-homolog (SEQ ID NO:23), Arabidopsis gi 11249511 (SEQ ID NO:44), soybean gi 5921926 (SEQ ID NO:45), soybean GE-homolog (SEQ ID NO:25), soybean GE-homolog (SEQ ID NO:21), and Arabidopsis gi 3831440 (SEQ ID NO:46). The boxed residues are predicted helical regions identified by the Bioscout DSC program (King and Sternberg (1996) Protein Sci 5:2298-2310). Other boxed elements include “SRS” or substrate-recognition-sites which are hypervariable sequences in the cytochrome P450 structure, “PPP” clusters of prolines often Pro-Pro-Gly-Pro in cytochrome P450s, “F-G loop” which is the substrate access channel (part of the conserved sequence motif of SEQ ID NO:83), the conserved “GXDT” the proton transfer groove involved in heme interaction and enzyme catalysis (part of the conserved sequence motif of SEQ ID NO:85), “EXXR” the K-helix motif conserved in all cytochrome P450s necessary for heme stabilization and core structure stability (part of conserved sequence motif of SEQ ID NO:88), and “FXXGXRXCXG” the conserved heme binding site with the cysteine that contacts the heme (part of the conserved sequence motif of SEQ ID NO:90).

The alignment of the sequences and comparison to related cytochrome P450 sequences provides a useful method for identifying motifs that are unique to GE-like cytochrome P450s. Many of the conserved sequence motifs found in SEQ ID NOs:80-91 are found at the edge of helical domains, or in SRS regions.

### Example 16

### Genetic Mapping of Maize GE Homolog to Loci Related to High Oil Seed Trait

High oil corn cultivars and rice giant embryo mutants share extensive similarities in their phenotypes. GE homologs were mapped to investigate the possible correlation between maize GE homologs and loci controlling high oil traits. Mapping was performed by finding polymorphic nucleotide sequences (SNPs) in the 3′UTR region. Gene specific primers were made to PCR amplify the gene from the genomic DNA of the mapping parents. The following primers were used for the amplification: 90F: AATTAACCCTCACTAMGGGCACCTGCTCTTCCACCAC (SEQ ID NO:108) and 91R: GTMTACGACTCACTATAGGGCGACTGCCCATTTCGTAGC (SEQ ID NO:109). The PCR products were directly sequenced by dye terminator chemistry, and the sequences were then aligned and analyzed for polymorphisms.

For the isolated nucleic acid fragment represented by zmGE1 (p0121.cformn62r), a polymorphism between the mapping parents G61/G39 was found at consensus position 73 with the nucleotide T in G61, but G in G39.

The location of polymorphisms are shown below (S corresponds to C or G, and K corresponds to G or T):

(SEQ ID NO:110)

CACCTGCTCTTCCACCACGCCATGGGCTTCGCGCCCTCSGGAGACGCGCA
CTGGCGCGGGCTCCGCCGCCTCKCCGCCAACCACCTGTTCGGCCCGCGCC
GCGTGGCGGGTGCCGCGCACCACCGCGCCTCCATCGGCGAGGCCATGGTC
GCCGACGTCGCCGCTGCCATGGCGCGCCACGGCGAGGTCCCTCTCAAGCG
CGTGCTGCATGTCGCGTCTCTCAACCACGTCATGGCCACCGTGTTTGGCA
AGCGCTACGACATGGGCAGCCGAGAGGGCGCCCTTCTGGACGAGATGGTG
GCCGAGGGCTACGACCTCCTGGGCACGTTCAACTGGGCTGATCAAC.

A sequencing primer close to the polymorphism was made in order to genotype 94 individuals in the mapping population by Pyrosequencing™ (Uppsala, Sweden; Rickert et al. (2002) BioTechniques 32:592-603). The sequencing primer, PY90R, was GGGCCGMCAGGTGGTTG (complementary sequence of positions 77-95 in SEQ ID NO:110, underlined above). The heritage score were then used to place the gene onto a core maize genetic map using MAPMAKER™ or JOINMAP™. Clone p0121.cformn62r was mapped onto the bottom of Chromosome 7, in the vicinity of the marker bnI8.39 in bin 7.04.

This map position was overlapped with one of the quantitative trait loci (QTL) that were associated with high seed oil.

The materials for QTL mapping were developed by crossing two lines, 49.007 and H31. 49.007 was a high oil inbred lined (about 20% kernel oil) developed from the ASKC28 population (Wang, SM. Lin YH and Huang AHC, 1984. Plant Phys., 76:837). H31 is a public line derived from the Illinois Low Oil (ILO) population that has very low kernel oil content (about 1%) (Quackenbush F W, Firch J G, Brunson A M and House L R. 1963. Cereal Chem. 40:250). From this cross, 180 F2:3 families were developed through two selfing generations. The F3 grain from individual F2 plants was evaluated for germ weight and other oil-related traits. One hundred kernels were shelled from the middle of each ear, dried to ˜5% moisture (40C for 4 d), weighed and oil content determined by NMR. Twenty germs were dissected from a random subsample of the 100 kernels to determine germ weight. Twenty seedlings of each F3 family were grown in greenhouse and the leaves of the seedlings were bulked on individual family basis. The leaf samples were lyophilized, ground into powder and used for DNA extraction. Genomic DNA was extracted by mini-CTAB method in a 96-well format. SSR markers were used in this mapping study. All genotypes were detected using ABI PRISM systems, which include the use of fluorescent end-label primers, gel electrophoresis on ABI377 DNA sequencer, peak detection and allele identification on GeneScan m and Genotyper™ software. A total of 89 polymorphic SSRs were used in mapping analysis. The linkage map was assembled by MAPMAKER and confirmed by MAPMANAGER. QTL analysis was carried out on mean value of each trait through composite interval mapping. QTL Cartographer was used to perform the analysis. Important parameters used in the analysis were:

Mapping function: Kosambi

QTL mapping method: Composite interval mapping

Significance threshold: LOD=2.5

Significance test for linear regression and backward stepwise linear regression: á=0.05

There appeared to be a QTL for the germ weight trait of high oil seed on chromosome 7. The putative QTL is in the region where EST p0121.cfrn62r (zmGEl) was mapped.

### Example 17

### Expression Analysis of Maize GE Homologs

In order to investigate a possible correlation between GE homologs and high oil traits, the expression pattern of zmGE2 was analyzed.

The expression study was conducted by comparing MPSS (Massively Parallel Signature Sequencing) data (Brenner et al. 2000. Nature Biotechnology 18:630-634; Brenner et al. (2000) Proc Natl Acad Sci USA 97:1665-1670), obtained from various corn tissues of different lines. MPSS data enabled a survey of expression levels in terms of looking at the abundance of particular cDNA clones among 1,000,000 clones for each library. The relative abundance of a particular tagged sequence, which is unique to a single cDNA, correlates with the relative level of accumulation of the corresonding RNA in that tissue. The expression of the GE homolog zmGE2 was detected, in all cultivars tested, by the presence of a specific tag sequence, GATCGATGGMCTGAGT (SEQ ID NO:111), in cDNAs from embryo tissues isolated 15 days after pollination. In corn cultivars with normal oil accumulation in seeds, zmGE2 was expressed with a frequency of 238/1,000,000 (238 parts-per-million or ppm) for the wild-type cultivar B73, and 263 ppm for the wild-type ASK cycle 0. In contrast, the expression of zmGE2 in high oil corn lines was reduced by more than 50%. In the high oil line, QX47, zmGE2 was expressed with a significantly lower frequency of 89 ppm. In another high oil line, ASK 28 cycles, the expression level was 113 ppm. A third high oil cultivar, IHO, gave an accumulation rate of 78 ppm. The reduction of expression is especially significant between ASK 0 (normal) and 28 cycles (high oil) because the two lines are derived from the same genetic background.

These data showed that one of the corn GE homologs, zmGE2, was substantially down-regulated in its expression in developing embryos of high oil lines. The result of the expression study confirmed that this GE homolog has a negative correlation with the high oil trait in corn seed. This is consistent with the rice result where mutations in GE genes result in enlarged embryos and high-oil phenotypes.

What is claimed is:

1. An isolated nucleotide fragment comprising a nucleic acid sequence selected from the group consisting of:
(a) a nucleic acid sequence encoding a cytochrome P450 polypeptide associated with controlling embryo/endosperm size during seed development having an amino acid identity of at least 61% based on the Clustal method of alignment when compared to a second polypeptide selected from the group consisting of SEQ ID NO:2, 7, 11, 19, 27, or 33;or
(b) a nucleic acid sequence encoding a cytochrome P450 polypeptide associated with controlling embryo/endosperm size during seed development having an amino acid identity of at least 65% based on the Clustal method of alignment when compared to a third polypeptide selected from the group consisting of SEQ ID NO:15, 17, 31, 93, 95, 97, or 99; or
(c) a nucleic acid sequence encoding a cytochrome P450 polypeptide associated with controlling embryo/endosperm size during seed development having an amino acid identity of at least 70% based on the Clustal method of alignment when compared to a fourth polypeptide selected from the group consisting of SEQ ID NO:9, 13, 23, 29, 35, or 4l; or
(d) a nucleic acid sequence encoding a cytochrome P450 polypeptide associated with controlling embryo/endosperm size during seed development having an amino acid identity of at least 77% based on the Clustal method of alignment when compared to a second polypeptide selected from the group consisting of SEQ ID NO:21, 25, 37, or 39; or
(e) the complement of (a) or (b) or (c) or (d).

2. The isolated nucleotide sequence of claim 1, or the complement thereof, which comprises at least one motif corresponding substantially to any of the amino acid sequences set forth in SEQ ID NOs:80-91 wherein said motif is a conserved subsequence.

3. The isolated nucleotide fragment of claim 1 or 2 wherein said fragment or part thereof is useful in antisense inhibition or co-suppression of a cytochrome P450 polypeptide associated with controlling embryo/endosperm size during seed development in a transformed plant.

4. An isolated nucleic acid fragment comprising a promoter wherein said promoter consists essentially of the nucleotide sequence set forth in SEQ ID NOs:3, 4, 104, or 105, or said promoter consists essentially of a fragment or subfragment that is substantially similar and functionally equivalent to the nucleotide sequence set forth in SEQ ID NOs:3, 4, 104, or 105.

5. A chimeric construct comprising the isolated nucleic acid fragment of claim 1 or 2 operably linked to at least one regulatory sequence.

6. A chimeric construct comprising the isolated nucleic acid fragment of claim 3 operably linked to at least one regulatory sequence.

7. The chimeric construct of claim 5 wherein said isolated nucleic acid fragment is operably linked to the promoter of claim 4.

8. The chimeric construct of claim 6 wherein said isolated nucleic acid fragment is operably linked to the promoter of claim 4.

9. A plant comprising in its genome the chimeric construct of claim 5.

10. A plant comprising in its genome the chimeric construct of claim 6.

11. A plant comprising in its genome the chimeric construct of claim 7.

12. A plant comprising in its genome the chimeric construct of claim 8.

13. Seeds obtained from the plant of claim 9.

14. Seeds obtained from the plant of claim 10.

15. Seeds obtained from the plant of claim 11.

16. Seeds obtained from the plant of claim 12.

17. Oil obtained from the seeds of claim 13.

18. Oil obtained from the seeds of claim 14.

19. Oil obtained from the seeds of claim 15.

20. Oil obtained from the seeds of claim 16.

21. The plant of claim 9 wherein said plant is selected from the group consisting of rice, corn, sorghum, millet, rye, soybean, canola, wheat, barley, oat, beans, and nuts.

22. The plant of claim 10 wherein said plant is selected from the group consisting of rice, corn, sorghum, millet, rye, soybean, canola, wheat, barley, oat, beans, and nuts.

23. The plant of claim 11 wherein said plant is selected from the group consisting of rice, corn, sorghum, millet, rye, soybean, canola, wheat, barley, oat, beans, and nuts.

24. The plant of claim 12 wherein said plant is selected from the group consisting of rice, corn, sorghum, millet, rye, soybean, canola, wheat, barley, oat, beans, and nuts.

25. Transformed plant tissue or plant cells comprising the chimeric construct of claim 5.

26. Transformed plant tissue or plant cells comprising the chimeric construct of claim 6.

27. Transformed plant tissue or plant cells comprising the chimeric construct of claim 7.

28. Transformed plant tissue or plant cells comprising the chimeric construct of claim 8.

29. The plant tissue or plant cells of claim 25 wherein the plant is selected from the group consisting of rice, corn, sorghum, millet, rye, soybean, canola, wheat, barley, oat, beans, and nuts.

30. The plant tissue or plant cells of claim 26 wherein the plant is selected from the group consisting of rice, corn, sorghum, millet, rye, soybean, canola, wheat, barley, oat, beans, and nuts.

31. The plant tissue or plant cells of claim 27 wherein the plant is selected from the group consisting of rice, corn, sorghum, millet, rye, soybean, canola, wheat, barley, oat, beans, and nuts.

32. The plant tissue or plant cells of claim 28 wherein the plant is selected from the group consisting of rice, corn, sorghum, millet, rye, soybean, canola, wheat, barley, oat, beans, and nuts.

33. A method of controlling embryo/endosperm size during seed development in plants which comprises:
(a) transforming a plant with the chimeric construct of claim 5;
(b) growing the transformed plant under conditions suitable for the expression of the chimeric construct; and
(c) selecting those transformed plants which produce seeds having an altered embryo/endosperm size.

34. A method of controlling embryo/endosperm size during seed development in plants which comprises:
(a) transforming a plant with the chimeric construct of claim 6;
(b) growing the transformed plant under conditions suitable for the expression of the chimeric construct; and
(c) selecting those transformed plants which produce seeds having an altered embryo/endosperm size.

35. A method of controlling embryo/endosperm size during seed development in plants which comprises:
(a) transforming a plant with the chimeric construct of claim 7;
(b) growing the transformed plant under conditions suitable for the expression of the chimeric construct; and
(c) selecting those transformed plants which produce seeds having an altered embryo/endosperm size.

36. A method of controlling embryo/endosperm size during seed development in plants which comprises:
(a) transforming a plant with the chimeric construct of claim 8;
(b) growing the transformed plant under conditions suitable for the expression of the chimeric construct; and
(c) selecting those transformed plants which produce seeds having an altered embryo/endosperm size.

37. A method to isolate nucleic acid fragments encoding polypeptides associated with controlling embryo/endosperm size during seed development which comprises:
(a) comparing SEQ ID NOs:2, 7, 9, 11, 13, 15, 17, 19, 21, 23, 25, 27, 29, 31, 33, 35, 37, 39, 41, 42, 43, 44, 45, 46, 47, 93, 95, 97, or 99 with other polypeptide sequences associated with controlling embryo/endosperm size during seed development;
(b) identifying the conserved sequences(s) or 4 or more amino acids obtained in step (a);
(c) making region-specific nucleotide probe(s) or oligomer(s) based on the conserved sequences identified in step (b); and
(d) using the nucleotide probe(s) or oligomer(s) of step (c) to isolate sequences associated with controlling embryo/endosperm size during seed development by sequence dependent protocols.

38. A method of mapping genetic variations related to controlling embryo/endosperm size and/or altering oil phenotype in plants comprising:
(a) crossing two plant varieties; and
(b) evaluating genetic variations with respect to
(i) a nucleic acid sequence selected from the group consisting of SEQ ID NO:1, 3, 4, 5, 6, 8, 10, 12, 14, 16, 18, 20, 22, 24, 26, 28, 30, 32, 34, 36, 38, 40, 92, 94, 96, 98, 100, 102, 104, or 105; or
(ii) a nucleic acid sequence encoding a polypeptide selected from the group consisting of SEQ ID NO:2, 7, 9, 11, 13, 15, 17, 19, 21, 23, 25, 27, 29, 31, 33, 35, 37, 39, 41, 42, 43, 44, 45, 46, 47, 80-91, 93, 95, 97, or 99;
in progeny plants resulting from the cross of step (a) wherein the evaluation is made using a method selected from the group consisting of: RFLP analysis, SNP analysis, and PCR-based analysis.

39. A method of molecular breeding to control embryo/endosperm size and/or altering oil phenotype in plants comprising:
(a) crossing two plant varieties; and
(b) evaluating genetic variations with respect to
(i) a nucleic acid sequence selected from the group consisting of SEQ ID NO:1, 3, 4, 5, 6, 8, 10, 12, 14, 16, 18, 20, 22, 24, 26, 28, 30, 32, 34, 36, 38, 40, 92, 94, 96, 98, 100, 102, 104, or 105; or
(ii) a nucleic acid sequence encoding a polypeptide selected from the group consisting of SEQ ID NO:2, 7, 9, 11, 13, 15, 17, 19, 21, 23, 25, 27, 29, 31, 33, 35, 37, 39, 41, 42, 43, 44, 45, 46, 47, 80-91, 93, 95, 97, or 99;
in progeny plants resulting from the cross of step (a) wherein the evaluation is made using a method selected from the group consisting of: RFLP analysis, SNP analysis, and PCR-based analysis.
